(19) United States
(12) Patent Application Publication
(10) Pub. No.: US 2022/0381772 A1
(43) Pub. Date: Dec. 1, 2022

(54) SYSTEMS AND METHODS FOR EVALUATING NK CELLS

(71) Applicant: The Regents of The University of California, Oakland, CA (US)

(72) Inventor: Anahid Jewett, Los Angeles, CA (US)

(21) Appl. No.: 17/629,948

(22) PCT Filed: Jul. 31, 2020
(86) PCT No.: PCT/US2020/044431

§ 371 Date: Jan. 25, 2022

Related U.S. Application Data
(60) Provisional application No. 62/881,626, filed on Aug. 1, 2019.

(51) Int. Cl. G01N 33/50; G01N 33/543; G01N 33/68

(52) U.S. Cl. CPC G01N 33/5011; G01N 33/5047; G01N 33/54386; G01N 33/6866

(57) ABSTRACT

The present application relates to natural killer (NK) cell-specific tests that are useful in determining the expansion potential and function of NK cells.

### CROSS-REFERENCE TO A RELATED APPLICATION

This application claims the benefit of U.S. Provisional Application No. 62/881,626, filed on Aug. 1, 2019, the entire contents of which are incorporated herein in their entirety by this reference.

### BACKGROUND OF THE INVENTION

The lymphocyte population in peripheral blood mononuclear cells (PBMCs) mainly constitutes T-cells, B-cells and, the natural-killer cells (NK cells). NK cells are known to play central defense against viral infection and killing tumor cells, and have been classified as effectors of innate immunity due to the lack of antigen specific cell surface receptors. T cells are known to mediate the cellular immunity mediating humoral immunity, provide adaptive immunity which work in close collaboration with the innate immune system. Human NK cells are defined phenotypically by the surface expression of CD56 and CD16, and by their lack of CD3 surface expression. About 90% of human NK cells are CD56dim CD16bright cells and found to be the major cytotoxic subset, whereas CD56bright CD16dim/− NK cells were found to secrete more cytokines. Major cytokines, secreted by NK cells are interferon-gamma (IFN-γ), tumor necrosis factor-alpha (TNF-α), TNF-β, granulocyte macrophage-colony stimulating factor (GM-CSF), interleukin-10 (IL-10), and IL-13.

NK cells isolated from the peripheral blood of cancer patients display phenotypic and functional alterations especially during advanced stage of cancer. It has been shown that freshly isolated tumor infiltrating NK cells are not cytotoxic to autologous tumors. T cells dysfunction has also been reported in cancer patients. Moreover, NK and T cells, especially NK cells obtained from the peripheral blood of patients with cancer have significantly reduced function particularly cytotoxic activity. Suppression of NK cells is mediated by downregulation of NK receptors in the tumor microenvironment. NK cells infiltration and cytotoxic activity of peripheral-blood lymphocytes has indirect co-relation the prognosis of cancer patients.

The major T-cell subpopulations are helper (CD4+) and cytotoxic (CD8+) T cells. The cellular immune responses that protect against tumors typically have been attributed to CD8+ T cells, CD8+ T cells are associated with chemo-response against the cancer. High numbers of T cells with CD8+ memory T cells, decreased proportions of tumor-infiltrating CD4+ T cells with high percentages of T-regulatory (Tregs) and, reversed CD4/CD8 ratios at tumor site were significantly associated with overall survival in patients with solid cancers. It has been shown that CD45RA+ T cells with high expression of CD62L and CCR7 have longer active life-span and are more effective against cancers in comparison to T memory cells. CD28 co-stimulation play crucial role in T cells anti-tumor and anti-microbial activity, lower surface expression of CD28 on cancer patients' T cells indicate their lower activity of T cells to fight against the cancer and the infection in those patients. Lower surface expression of CD127 on the surface of T cells has been shown to be influenced by the presence of cancer and infections.

Natural killer (NK) cells lyse and differentiate cancer stem cells/undifferentiated tumors with lower expression of MHC class I, CD54 and B7H1 and higher expression of CD44. Medium and high cytotoxic activity of peripheral-blood lymphocytes are associated with reduced cancer risk, and high NK-cell infiltration of the tumor is associated with a better prognosis, whereas low activity is associated with increased cancer risk.

Lower MHC-class I expression on cancer stem cells (CSCs)/poorly differentiated tumors might favor their survival, and explain their limited effectiveness to T-cell based immunotherapies in cancer patients. CSCs are excellent targets of NK cell-mediated cytotoxicity, whereas their differentiated counterparts are significantly more resistant. Furthermore, de-differentiation of tumors resulted in their increased susceptibility to NK cell-mediated cytotoxicity. It is known that cytotoxic function of primary NK cells is suppressed after their interaction with CSCs/stem cells. NK cells, as a result of CD16 receptor cross-linking or interaction with CSCs/undifferentiated tumors, undergo split-anergy, a key event in which NK-cytotoxicity is lost but a greater secretion of IFN-γ is triggered which promote an increase in the differentiation antigen expression of MHC-class I, CD54 and PD-L1 on tumors which has recently been shown to correlate with effectiveness of anti-PD-1 therapy. Indeed, overall higher levels of circulating NK cells are associated with better prognosis in cancer patients.

The anti-tumor activities of NK cells make NK cells an attractive candidate for use in immunotherapies for cancer, and various NK cell-based therapies have been or currently being evaluated in the clinic for malignant tumors. However, NK cells from cancer patients show a range of anti-tumor activities because cancers develop mechanisms to induce defective NK cells. Accordingly, the NK cells from cancer patients often do not function optimally and are not suitable for use in NK cell-based immunotherapies. For example, NK cell cytotoxic activity in peripheral blood of cancer patients is reduced, and also the expression of NK cell activating receptors were diminished even at the early stages of cancer and are further reduced in advanced disease. Defect in NK cell function is seen both at the pre-neoplastic and neoplastic stages of pancreatic cancer.

Accordingly, there is a great need for methods to assess accurately the expansion potential and function of NK cells, and the suitability of NK cells for use in NK cell-based immunotherapies.

### SUMMARY OF THE INVENTION

The present invention is based, at least in part, on the discovery that the methods presented herein provide surprisingly sensitive and specific ways to assess NK cell expansion potential and function. These methods are useful in determining the function and expansion potential of NK cells of healthy patients as well as diseased patients. In some embodiments, the patients have cancer, such as pancreatic cancer or oral cancer (e.g., oral squamous carcinoma). The methods presented herein are also useful in determining the suitability of NK cells for use in immunotherapy to treat diseased patients. Such NK cells may be autologous or allogenic to the patient. In some embodiments, the NK cells may be expanded, modified, and/or activated in vitro, ex vivo, or in vivo before and/or after being assessed using the methods described herein. In certain embodiments, the expansion, modification, and/or activation of NK cells involve contacting the NK cells with cytokines, antibodies, osteoclasts, exogenous nucleic acids encoding genes that are important for NK function, such as CD16 receptor, and/or any combination thereof. Such exogenous nucleic acids may be transduced by viral vectors including but not limited to lentivirus or adeno-associated virus (AAV).

The methods described herein assess multiple functions of NK cells, the combination of which has not been previously predicted to be useful or essential. In some embodiments, the methods assess the cytotoxic function of NK cells, including (i) the direct cytotoxicity against cells that are supringly sensitive to NK cells, such as oral squamous cancer stem cells (OSCSC) and/or Mia-Paca-2 (MP2), and/or (ii) antibody-dependent cellular cytotoxicity (ADCC) activity of NK cells, which is particularly useful in assessing the NK cell cytotoxicity against differentiated tumor cells. In some embodiments, the methods assess the amount of IFN-γ produced by the NK cells, e.g., by ELISA and/or ELISPOT. In some embodiments, the methods assess the ability of IFN-γ produced by the NK cells to induce differentiation of tumor cells. In some embodiments, these methods, either alone or in combination, further comprise measuring other important aspects of NK cell function, including (i) the amount and/or function of a CD16 receptor on the NK cells, (ii) the ability of the NK cells to expand CD8+ T cells, and/or measuring the expression level of at least one biomarker selected from CD44, CD54, MHC class I, PD-L1 (B7H1), and MICA/B. In some embodiments, the methods further comprise administering to a subject at least one selected from autologous NK cells, allogeneic NK cells, and NK cell-expanded CD8+ T cells, optionally wherein NK cells have been expanded, modified, and/or activated. In some embodiments, the subject is a mammal, such as a human.

### BRIEF DESCRIPTION OF THE DRAWINGS

FIG. 1. shows that OSCSCs are sensitive and specific targets of NK cells. OSCCs or OSCSCs were seeded at 1×10⁵ cells/well in 24-well plate for 24 h prior to the addition of highly purified NK cells pre-treated with IL-2 (1000 units/ml) for 24 h. NK cells were added to tumor cells at 2:1 effector to target ratio. At time 0 when NK cells were added to the tumor culture, a final concentration of 10 μg/ml of propidium iodide (PI) was also added. The cells were then subsequently tracked for over 72 h using time-lapse microscopy with Nikon Eclipse Ti-E inverted microscope fitted with a culture chamber to provide cells with a stable temperature of 37° C. with 5% CO2. An image was taken every 15 min and a representation is shown at day 1, day 1½, and day 3. OSCSCs but not OSCCs which are lysed take up PI and appear orange in the time-lapse (A). NK cells were left untreated or treated with IL-2 (1000 units/ml), anti-CD16 mAb (3 μg/ml), or a combination of IL-2 (1000 units/ml) and anti-CD16 mAb (3 μg/ml) for 18 h before they were added to ⁵¹Cr-labeled OSCSCs and OSCCs. NK cell-mediated cytotoxicity was determined using a standard 4 h ⁵¹Cr release assay and the lytic units 30/10⁶ cells were determined using inverse number of NK cells required to lyse 30% of the target cells×100 (B). NK cells were treated as described in (B) and each NK sample was either cultured in the absence or presence of OSCSCs and OSCCs at an NK cell to target cell ratio of 0.5:1. After an overnight incubation, the supernatants were removed from the co-cultures and the levels of IFN-γ secretion were determined using specific ELISAs. One of minimum three representative experiments is shown in each of (C). The surface expression of MHC class I and CD44 on OSCCs and OSCSCs were assessed with flow cytometric analysis after staining with the respective PE-conjugated antibodies. Isotype control antibodies were used as control. The numbers on the right hand corner are the percentages and the mean channel fluorescence intensities for each histogram (D). The surface expression of CD338 on OSCCs and OSCSCs was assessed by flow cytometric analysis after staining with PE-conjugated CD338 (right graphs in the histogram). Isotype control antibodies were used as control (left graphs in the histograms) (E). OSCCs and OSCSCs were left untreated or treated with 10-80 μg/ml of cisplatin for 18 h, after which the tumor cells were washed with 1×PBS, detached, and stained with propidium iodide (PI) and percent cell death was determined using flow cytometric analysis (F).

FIG. 2. shows increased MICA/B expression on differentiated Oral tumors and increased ability of NK cells to mediate ADCC against OSCCs but not OSCSCs. Surface expression of MICA/MICB on OSCCS, OCSCSs, and NK cell supernatant-differentiated OSCSCs was assessed using flow cytometric analysis after staining with PE-conjugated anti-MICA/MICB antibodies. PE-conjucated Isotype control antibodies were used as controls (FIG. 2A). Freshly purified NK cells from healthy donors were left untreated, treated with IL-2 (1,000 U/mL) or combination of IL-2 and anti-CD16 mAb (3 μg/ml), for 18 hours. OSCCS (FIG. 2B), and OSCSCs (FIG. 2C) were labeled with ⁵¹Cr, and then left untreated or treated with anti-MICA/MICB antibody (5 μg/ml) for 30 minutes. The unbound antibodies were removed by washing the tumors and cytotoxicity against untreated and MICA/B treated OSCCs (FIG. 2B) and OSCSCs (FIG. 2C) was determined using the standard 4-hour ⁵¹Cr release assay.

FIG. 3. shows the stage of differentiation in pancreatic tumors correlates with susceptibility to NK cell mediated cytotoxicity. The surface expression of CD44, CD54, and MHC-class I on multiple pancreatic cell lines were assessed with flow cytometric analysis after staining with the respective PE-conjugated antibodies. Isotype control antibodies were used as control (A). Freshly isolated NK cells were left untreated or treated with anti-CD16mAb (3 μg/ml), IL-2 (1000 U/ml) or the combination of anti-CD16mAb (3 μg/ml) and IL-2 (1000 U/ml) for 18 h before they were added to ⁵¹Cr labeled MP2, Panc-1, BXPC3, HPAF, Capan and PL12. NK cell-mediated cytotoxicity was determined using a standard 4-hour ⁵¹Cr release assay and the lytic units 30/10⁶ cells were determined using inverse number of NK cells required to lyse 30% of the target cells×100. Please note we did not observe any killing of the tumor cells when anti-CD16 mAB treated NK cells were used in the cytotoxicity assay in the triplicates (B). One of the eight representative experiments is shown in the figure.

FIG. 4 shows that MP2, PL.12, and Capan tumors (1×10⁵ tumors/well) were treated with or without Paclitaxel for 18-20 hours before the viability of cells was determined using propidiun iodide staining. P-values of <0.05 were obtained for differences between MP2 vs. PL12 and Capan at the concentrations of 10 nM, 200 nM, 600 nM, 1000 nM and 10 g of Paclitaxel (n=2 per each experimental condition).

FIG. 5 shows increased MICA/B expression on differentiated pancreatic tumors and increased ability of NK cells to mediate ADCC against PL-12 but not MP2 tumors. Surface expression of MICA/MICB on differentiated PL-12, poorly differentiated MP2, and split-anergized NK cell supernatant-differentiated MP2s was assessed using flow cytometric analysis after staining with PE-conjugated anti-MICa/MICB antibodies. PE-conjucated Isotype control antibodies were used as controls (FIG. 5A). Freshly purified NK cells from healthy donors were left untreated, treated with IL-2 (1,000 U/mL) or combination of IL-2 and anti-CD16 mAb (3 μg/mil), for 18 hours. PL-2, and MP2s were labeled with 51Cr, and then left untreated or treated with anti-MITCA/MICB antibody (5 μg/ml) for 30 minutes. The unbound antibodies were removed by washing the tumors and cytotoxicity against untreated and MICA/B treated PL12 (FIG. 5B) and MP2s (FIG. 5C) were determined using the standard 4-hour 51Cr release assay. Freshly purified NK cells from healthy donors were left untreated, treated with IL-2 (1,000 U/mL) or combination of IL-2 and anti-CD16 mAb (3 μg/ml), for 18 hours and used along with IL-2 treated NK92 and NK92V (transfected with CD 16 gene) in the presence and absence of anti-CD16mAb antibody (3 μg/ml). PL-12, and MP2s were labeled with 51Cr, and then left untreated or treated with anti-MICA/MICB antibody (5 μg/ml) for 30 minutes. The unbound antibodies were removed by washing the tumors and primary NK as well as NK92 mediated cytotoxicity against untreated and MICA/B treated PL12 (FIG. 5D) and MP2s (FIG. 5E) were determined using the standard 4-hour 51Cr release assay.

FIG. 6. NK cells, CD3+ T cells, CD4+ T cells, CD8+ T cells and γδ cells were all sorted from the peripheral blood and activated with IL-2 (1000 u/ml) before they were added to 51 Cr labeled OSCSCs in a standard 4 hour 51Cr release assay (FIG. 6A). Cord blood NK cells were generated from sorted CD34+ cells from the cord blood and differentiated to NK cells according to the established protocols in the literature and compared to primary NK cells and super-charged NK cells cytotoxicity against 51Cr labeled OSCSCs (FIG. 6B). iPSC derived NK cells were made according to the established protocols and the effect were compared to super-charged NK cells (FIG. 6C).

FIG. 7 shows functional loss of NK cells obtained from peripheral blood of cancer patients. Human PBMCs were isolated from the peripheral blood of the healthy individuals and cancer patients and NK cells were purified and treated with IL-2 (1000 U/ml) (FIGS. 7A and 7B) or IL-2 (1000 U/ml) and anti-CD16mAb (3 μg/ml) (FIG. 7B) for 18 hours before they were added to ⁵¹Cr labeled oral squamous cell carcinoma stem cells (OSCSCs) at various effectors to target ratios. The lytic units 30/10⁶ cells were determined using the inverse number of NK cells required to lyse 30% of OSCSCs×100 (D).

FIG. 8. To generate super-charged NK cells the following protocol was employed. Monocytes were purified from healthy individuals' PBMCs and cultured in alpha-MEM media containing M-CSF (25 ng/ml) and RANKL (25 ng/ml) for 21 days to generate osteoclasts (OCs). Purified NK cells (1×10⁶ cells/ml) from healthy individuals were treated with the combination of IL-2 (1000 U/ml) and anti-CD16mAb (3 μg/ml) for 18 hours before they were cultured with OCs generated from healthy individual monocytes in the presence of sAJ2 at 1:2:4 ratios (OCs:NK:sAJ2). After 15 days of expansion, the numbers of expanded NK cells were determined using microscopy and compared to those expanded by monocyte expanded NK cells and irradiated PBMC expanded NK cells which are the gold standard expansion methodologies used previously (A). Freshly purified NK cells from the healthy individuals were treated and co-cultured with OCs to generate super-charged NK cells as described in FIG. 8A. Cytotoxicity of day 15 cultured super-charged NK cells was determined using standard 4-hour ⁵¹Cr release assay against OSCSCs and compared to those obtained from monocyte expanded NK cells or irradiated PBMC expanded NK cells. The lytic units 30/10⁶ cells were determined using the inverse number of NK cells needed to lyse 30% of the tumors×100 (B). Freshly purified NK cells from the healthy individuals were treated and co-cultured with OCs to generate super-charged NK cells as described in FIG. 8A, and the supernatants were harvested from the day 15 co-cultures and IFN-γ secretion was determined using single ELISAs (C).

FIG. 9A-FIG. 9B. PBMCs were isolated from blood, and purified populations of NK cells were obtained from both healthy donor and that of pancreatic cancer patient (P52) and left untreated or treated with IL-2 (1000 u/ml), IL-2 (1000 u/ml) in combination with anti-CD16 mAb (3 mg/ml) and IL-2 (1000 u/ml) with anti-CD3 and anti-CD28 and IL-2 (1000 u/ml) with sAJ2 (2:1 bacteria to PBMC or NK cells) for 18-24 hours before the supernatants were removed and subjected to ELISA and cells (50,000 per well) were used for Elispot assay. The IFNg spots were counted by the Immunospot CTL instrument.

FIG. 10. Purified NK cells from healthy donor and that of pancreatic cancer patient were treated with IL-2 (1000 u/ml) with anti-CD16mAb (3 mg/ml) overnight and supernatants were harvested and the levels of IFN-g were determined. The same amounts of IFN-g secreted in the supernatants of NK cells from healthy donor and that of patient were added to OSCSCs and allowed differentiation of tumors to proceed for 5 days. After 5 days the OSCSCs were washed and the levels of CD44, MHC class I, CD54 and B7HI were determined on OSCSCs differentiated by IFN-g secreted from the NK cells of healthy donor and that of the pancreatic cancer patient.

FIG. 11A-FIG. 11F show decreased numbers of PBMCs and functional loss of NK cells obtained from peripheral blood of cancer patients. Human PBMCs were isolated from the peripheral blood (30 ml) of the healthy individuals (n=14) and cancer patients (n=14), and the numbers of cells were determined using microscopy (A). Human PBMCs were isolated from the peripheral blood of the healthy individuals (n=9-12) and cancer patients (n=9-12), and equal numbers (2×10⁵ cells) of PBMCs were used to determine the percentages of CD16 (n=12), CD56 (n=12), CD3 (n=12), CD19 (n=9), CD14 (n=10) and CD11b (n=9) subsets within CD45+ immune cells using flow cytometric analysis (B). Human NK cells were isolated from healthy individuals (n=9) and cancer patient's (n=9) PBMCs as described in Materials and Methods. Purified NK cells (1×10⁶ cells/ml) were left untreated and treated with IL-2 (1000 U/ml) for 18 hours before the supernatants were harvested and IFN-γ secretion was determined using single ELISA (C). NK cells isolated and treated as described in FIG. 1C, and were added to ⁵¹Cr labeled oral squamous cell carcinoma stem cells (OSCSCs) at various effectors to target ratios. NK cell-mediated cytotoxicity using a standard 4-hour ⁵¹Cr release assay against the oral squamous cell carcinoma stem cell line (OSCSCs). The lytic units 30/10⁶ cells were determined using the inverse number of lymphocytes required to lyse 30% of OSCSCs×100 (n=9 for each experiment condition) (D). Highly purified NK cells isolated from individuals (n=4) and cancer patients' (n=4) PBMCs were treated (1×10⁶ cells/ml) with IL-2 (1000 U/ml) for 18 hours before the supernatants were harvested and ran with multiplex cytokine array kit to determine IFN-γ, IL-12p70, IL-6, TNF-α, IL-5 and IL-4 secretion (E). Serum were obtained from peripheral blood of healthy donors (n=5) or cancer patients (n=8) and analyzed for the levels of cytokines, chemokine and growth factors using multiplex array kit (F).

FIG. 12 shows that osteoclast-expanded super-charged NK cells from cancer patients have much lower capacity to expand, or mediate cytotoxicity and secrete IFN-γ compared to healthy individuals. Monocytes were purified from healthy individual PBMCs and cultured in alpha-MEM media containing M-CSF (25 ng/ml) and RANKL (25 ng/ml) for 21 days to generate osteoclasts (OCs). Purified NK cells (1×10⁶ cells/ml) from healthy individuals (n=70) and cancer patients (n=70) were treated with the combination of IL-2 (1000 U/ml) and anti-CD16mAb (3 μg/ml) for 18 hours before they were cultured with OCs generated from healthy individual monocytes in the presence of sAJ2 at 1:2:4 ratios (OCs:NK:sAJ2). After 6, 9, 12 and 15 days of co-culture, the numbers of expanded lymphocytes were determined using microscopy (A). Freshly purified NK cells from the healthy individuals (n=16) and cancer patients (n=16) were treated and co-cultured with OCs as described in FIG. 12A. Cytotoxicity of day 15 cultured NK cells was determined using standard 4-hour ⁵¹ Cr release assay against OSCSCs. The lytic units 30/10⁶ cells were determined using the method described in FIG. 1D (B). Freshly purified NK cells from the healthy individuals (n=63) and cancer patients (n=63) were treated and co-cultured with OCs as described in FIG. 12A, and the supernatants were harvested from the days 6, 9, 12 and 15 co-cultures and IFN-γ secretion was determined using single ELISAs (C). Freshly purified NK cells from the healthy individuals (n=21) and cancer patients (n=21) were treated and co-cultured with OCs as described in FIG. 12A. After 6, 9, 12 and 15 days of co-cultures, the numbers of expanded NK cells were counted using microscopy and the supernatants were harvested to determine the IFN-γ secretion using single ELISA. IFN-γ secretion was assessed based on per 1 million cell counts (D).

FIG. 13 shows the decreased percentages of CD4+ T cells and an increase in percentages of CD8+ T cells when determined within cancer patients' PBMCs as compared to healthy individuals' PBMCs. Monocytes were purified from healthy individual PBMCs and cultured in alpha-MEM media containing M-CSF (25 ng/ml) and RANKL (25 ng/ml) for 21 days to generate osteoclasts (OCs). Purified NK cells (1×10⁶ cells/ml) from healthy individuals (n=28) and cancer patients (n=28) were treated with the combination of IL-2 (1000 U/ml) and anti-CD16mAb (3 μg/ml) for 18 hours before they were cultured with OCs generated from healthy individual monocytes in the presence of sAJ2 at 1:2:4 ratios (OCs:NK:sAJ2). Purified T cells from the healthy individuals (n=28) and cancer patients (n=28) were treated with the combination of IL-2 (100 U/ml) and anti-CD3 (1 μg/ml)/CD28mAb (3 μg/ml) for 18 hours before they were co-cultured with OCs in the presence of sAJ2 at 1:2:4 ratios (OCs:T:sAJ2). After 6, 9, 12 and 15 days of co-culture, the percentages of CD4 and CD8 were analyzed within CD3+ immune cells expanded by the osteoclasts alone and those with NK and osteoclasts were determined using flow cytometry (A), and the ratio of CD4+ to CD8+ T cells were determined (B).

FIG. 14 shows that OC-expanded NK cell immunotherapy increased CD8+ T cells, IFN-γ secretion, and NK cell-mediated cytotoxicity in BM, spleen, and peripheral blood of hu-BLT mice. Immunotherapy with OC-expanded NK cells in tumor-bearing hu-BLT mice increased CD8+ T cells in BM, spleen, and peripheral blood and, resulted in increased IFN-γ secretion and elevated NK cell-mediated cytotoxicity in those tissue compartments. Reconstituted BLT (levels and lineages of T cells comparable to the healthy donors) were orthotopically injected with 1×10⁶ of human OSCSCs into the floor of the mouth (A). 1-2 weeks after the tumor implantation, mice were i.v injected with day 12 OC-expanded human NK cells, NK cells were treated and co-cultured with OCs as described in FIG. 12A. Disease progression and weight loss was monitored for another 3-4 weeks (n=3). At the end of experiment hu-BLT mice were sacrificed, the spleens, BM and peripheral blood were harvested, and single cell suspensions were obtained, and cells obtained from each tissue were treated with IL-2 (1000 U/ml) and cultured for 7 days. Surface expression of CD3 and CD8 was analyzed at days 7 of BM culture (n=3 for each experimental condition) (B), spleen culture (n=3 for each experimental condition) (E), PBMC culture (n=2 for each experimental condition) (H). The supernatants were harvested from the cultures on day 7 of BM culture (n=3 for each experimental condition) (C), spleen culture (n=3 for each experimental condition) (F), PBMC culture (n=2 for each experimental condition) (I), and IFN-γ secretion was determined using single ELISA (C, F, 1). NK-cells mediated cytotoxicity against OSCSCs of 7 days BM culture (n=3 for each experimental condition) (D), spleen culture (n=3 for each experimental condition) (G), PBMC culture (n=2 for each experimental condition) (J) was determined using standard 4-hour ⁵¹Cr release assay against OSCSCs. The lytic units 30/10⁶ cells were determined using the method described in FIG. 1D (D, G, J). Blood was collected post-mortem by cardiac puncture and serum samples were harvested and analyzed for IFN-γ, IL-6, ITAC, IL-8 and GM-CSF using multiplex arrays (K).

FIG. 15 shows that CD8+ T cells expanded by super-charged NK cells secrete higher levels of cytokines when compared to OC expanded CD8+ T cells. Freshly purified NK cells from the healthy donors were treated and co-cultured with OCs as described in FIG. 12A. On day 12 of the culture, CD8-T cells were isolated using isolation kits, and were treated with IL-2 (100 U/ml) and anti-CD3 (1 μg/ml)/CD28mAb (3 μg/ml) for 18 hours. In separate culture CD8+ T cells were purified from human PBMCs and were treated with IL-2 (100 U/ml) and anti-CD3 (1 μg/ml)/CD28mAb (3 μg/ml) for 18 hours before they were cultured with OCs (1:2:4; OCs:T:sAJ2). On day 12 of culture, CD8+ T cells were isolated from the culture using isolation kits, and were treated with IL-2 (100 U/ml) and anti-CD3 (1 μg/ml)/CD28mAb (3 μg/ml) for 18 hours. The supernatants were harvested from both CD8+ T cell cultures at the same time and analyzed for the levels of cytokines, chemokines and growth factors using multiplex array kit, and the fold increase of secretion levels of CD8+ T cells sorted from OCs expanded NK cells were compared to CD8+ T cells cultured with OCs.

FIG. 16 shows that activation through CD16 receptor does not trigger IFN-g secretion from the cancer patients' PBMCs and NK cells. PBMCs and NK cells were isolated from healthy individuals and those of cancer patients and treated with different activation agents as indicated in the figure. The treatments were carried out as described above and after an overnight incubation the supernatants were removed and subjected to ELISA (A) and cells were added to the plates and Elispot carried out (B). For Elispot experiments NK cells as well as monocytes were purified and co-cultured in the presence of activators overnight before the plates were developed for IFN-g spots. The extent of increase in spots is shown for each well in the figure. 1:1 NK to monocyte ratio was used in criss cross experiments using NK cells from patients with monocytes from both healthy as well as patients as well as NK cells from healthy donors with monocytes from both healthy and patients to determine whether NK or monocytes or both are defective in their function.

FIG. 17 shows the characteristics of stem-like/poorly differentiated and well differentiated tumors.

FIG. 18 shows that poorly differentiated/stem-like MP2 tumors formed larger tumors in the pancreas and metastasized to liver and lungs whereas, differentiated PL12 tumors formed smaller tumors in the pancreas and did not metastasize to liver.

FIG. 19A-FIG. 19B show a lack of tumor growth, and long-term survival of NSG mice after orthotopic implantation of NK-differentiated MP2 tumors in pancreas; MP2 tumors were differentiated by the NK-supernatants as described in Example 9. MP2 tumors (3×10⁵) (n=3) (panel a), patient-derived differentiated PL12 (2×10⁶) (n=3) (panel b), and NK-differentiated MP2 tumors (diff-MP2) (5×10⁵) (n=3) (panel c), were implanted into the pancreas of NSG mice and tumor growth were determined in 4 weeks for MP2 tumors and 12 weeks for PL-12 and diff-MP2 tumors (FIG. 19A). The rates of survival of the mice in panels a, b and c (FIG. 19B) as well as tumor metastasis to liver (FIG. 18) were determined after euthanasia.

FIG. 20A-FIG. 20D show single injection of super-charged NK cells inhibited tumor growth and increased immune cells in the pancreas in hu-BLT mice. Hu-BLT mice were generated as described in the Example 9 and as depicted in the figure S2B in supplementary file, and they were implanted with 1×10⁶ tumors in the pancreas, and, injected with 1.5×10⁶ super-charged NK cells via tail vein after one to two weeks (FIG. 20A) and disease progression was monitored. After 6-7 weeks mice were euthanized and pictures of the tumors within the pancreas were taken post-mortem in mice injected with tumor in the absence of super-charged NK cells (panel a), tumor-bearing mice injected with super-charged NK cells (panel b), and NK-differentiated tumors (panel c) (n=5/each experimental condition); one representative experiment is shown in the figure (FIG. 20B). Pancreas was dissociated as described in the Example 9 and the single cells (1×10⁶ cells/ml) were used to determine the percentages of huCD45+CD3+(FIG. 20C) and huCD45+CD16+ cells (FIG. 20D) (n=3/each experimental condition).

FIG. 21A-FIG. 21O show single injection of super-charged NK-cells with/without feeding with AJ2 inhibited tumor growth due to differentiation of tumors in hu-BLT mice. Implantation of tumor cells in the pancreas and tail vein injection of super-charged NK cells and feeding with AJ2 (5×10⁹) were carried out as depicted in the figure (FIG. 21A), and disease progression was monitored. Mice were sacrificed, and pancreatic tumors were harvested and weighed (n=4/each experimental condition) (FIG. 21B). Implantation of tumor cells in the pancreas and tail vein injection of super-charged NK cells were carried out as depicted in FIG. 21A, and at the time of sacrifice mice were bled and the levels of IFN-γ in the serum were determined using multiplex array (n=3) (FIG. 21C). Procedures were carried out as depicted in FIG. 21A, FIG. 27A and FIG. 28C before pancreatic tumors were harvested and weighed (n=3) (FIG. 21D). Upon sacrifice, pancreatic tumors were harvested and single cell suspensions were prepared as described in Example 9. The same numbers of pancreatic tumor cells from each mouse were cultured, and the pictures of cultured tumors were taken on day 7. One of the four representative experiments is shown in the figure (FIG. 21E). Procedures were carried out as described in FIG. 21A using injections of allogeneic or autologous super-charged NK cells. Pancreatic tumors were resected and single-cell suspensions were prepared and tumor growth were assessed (n=9 to 12/each experimental condition) using identical numbers of tumors cultured from each mouse tumor (FIG. 21F). Hu-BLT mice were implanted with MP2 tumors and injected with NK cells or implanted with NK-differentiated tumors as described in FIG. 21A, and FIG. 27A and FIG. 28C. At the end of the experiment pancreatic tumors were harvested and tumor growth was assessed on days 7, 11 and 14, and on day 7 attached tumors from each well were counted and equal numbers of tumors from each group were re-cultured and tumor growth in each well was determined every 3 days (n=12/each experimental condition, one representative experiment is shown in the figure) (FIG. 21G). Hu-BLT mice were implanted with tumors and injected with super-charged NK cells, as described in FIG. 21A. Tumors were resected, and single cell cultures were prepared and cultured for 7 days, after which percentages of human CD45, CD94, CD56, NKG2D, and DNAM within the tumors were determined after staining with antibodies, followed by flow cytometric analysis (FIG. 21H, FIG. 21I, FIG. 27C, FIG. 27D). Procedures were carried out as described in FIG. 21A and FIG. 27A. Pancreata were removed, and single cell cultures were prepared and equal numbers of pancreatic cells were cultured until day 7 or 11 and the levels of IFN-γ (FIG. 21J) and IL-6 (FIG. 21K) were determined in culture supernatants (n=4/each experimental condition). Hu-BLT mice were implanted with MP2 tumors and injected with NK cells and fed with AJ2 as described in FIG. 21A. Upon sacrifice, tumors were resected, and single cell cultures were prepared, and equal numbers of tumors were cultured on day 7 or 11 and the levels of IFN-γ were determined in culture supernatants (n=2/each experimental condition) (FIG. 21L). Procedures were carried out as described in FIG. 21A. Tumors were resected, and single cell cultures were prepared and surface expressions of MHC-class I, B7H1 and CD54 were determined on tumors after culture (n=4/each experimental condition) (FIG. 21M). NK cells (1×106 cells/mL) from healthy individuals were treated with IL-2 (1000 U/mL) for 18-24 h before they were added to 51Cr labeled tumors obtained from mice implanted with different tumors and/or injected with NK cells as described in FIG. 21A at various effector to target ratios. NK cell mediated cytotoxicity was determined using 4 h 51Cr release assay, and the lytic units 30/106 cells were determined using inverse number of NK cells required to lyse 30% of the tumor-cells×100 (n=3/each experimental condition) (FIG. 21N and FIG. 21O). The following symbols represent the levels of statistical significance within each analysis, *** (p-value <0.001), ** (p-value 0.001-0.01), * (p-value 0.01-0.05).

FIG. 22A-FIG. 22L show injection of super-charged NK-cells with/without feeding with AJ2 restored and increased IFN-γ secretion and/or cytotoxic function of NK cells from different tissues of tumor-bearing hu-BLT mice. Procedures were carried out as described in FIG. 21A, FIG. 27A and FIG. 28C. Upon sacrifice, PBMCs were isolated from blood and treated with IL-2 (1000 U/mL) before they were used in cytotoxicity assay against OSCSCs using 4 h ⁵¹Cr release assay. Lytic units 30/10⁶ cells were determined using inverse number of NK cells required to lyse 30% of the target cells×100 (FIG. 22A). Procedures were carried out as described in FIG. 21A, and FIG. 27A and FIG. 28C before the PBMCs were isolated and treated with (1000 U/mL) and the supernatants were harvested and IFN-γ secretion was determined using ELISA (n=4/each experimental condition) (FIG. 22B and FIG. 22C). Procedures were carried out as described in FIG. 21A, and FIG. 27A and FIG. 28C before spleens were harvested, and single-cell suspensions were prepared. Splenocytes were treated with IL-2 (1000 U/mL) before they were used for cytotoxicity against OSCSCs using 4 h ⁵¹Cr release assay. Lytic units 30/10⁶ cells were determined using inverse number of NK cells required to lyse 30% of the target cells×100 (n=4/each experimental condition) (FIG. 22D). Procedures were carried out as described in FIG. 21A, and FIG. 27A and FIG. 28C before the supernatants were harvested from day 3 or 7 cultures of splenocytes, and IFN-γ secretion was determined using ELISA (n=5/each experimental condition) (FIG. 22E and FIG. 22F). Procedures were carried out as described in FIG. 21A and NK-enriched cells were isolated from splenocytes and were cultured with IL-2 (1000 U/mL) before they were used for cytotoxicity against OSCSCs using 4 h ⁵¹Cr release assay. Lytic units 30/10⁶ cells were determined using inverse number of NK cells required to lyse 30% of the target cells×100 (n=4/each experimental condition) (FIG. 22G). Supernatants were harvested from day 3 or 7 NK-enriched cultures and IFN-γ secretion was determined using ELISA (n=6/each experimental condition) (FIG. 22H). Procedures were carried out as described in FIG. 21A and the CD3+ T-cells were isolated from splenocytes and were cultured with IL-2 (100 U/mL), and on day 3 or day 7 the supernatants were harvested and IFN-γ secretion was determined using ELISA (n=4/each experimental condition) (FIG. 22I). Procedures were carried out as described in FIG. 21A, and FIG. 27A and FIG. 28C and BM cells were harvested and treated with IL-2 (1000 U/mL) for 7 days before they were used for cytotoxicity against OSCSCs using 4 h ⁵¹Cr release assay. Lytic units 30/10⁶ cells were determined using inverse number of NK cells required to lyse 30% of the target cells×100 (n=4/each experimental condition) (FIG. 22J). Supernatants were harvested on day 3 or day 7 of BM cultures and IFN-γ secretion was determined using ELISA (n=6/each experimental condition) (FIG. 22K and FIG. 22L). The following symbols represent the levels of statistical significance within each analysis, *** (p-value <0.001), ** (p-value 0.001-0.01), * (p-value 0.01-0.05).

FIG. 23 shows MP2 tumors were differentiated with NK supernatants in the presence and absence of anti-IFN-γ and anti-TNF-α as described in Example 9 for a period of 5 days before they were washed and treated with/without NAC (20 nM) for 24 h, followed by paclitaxel treatment from 10 nM to 600 nM for 18-24 h. The viability of cells was determined by staining with propidium iodide (n=3/each experimental condition).

FIG. 24A-FIG. 24D show that monocytes or osteoclasts from tumor-bearing mice injected with NK cells or implanted with NK-differentiated MP2 tumors induced increased IFN-γ secretion by the NK cells when compared to those of tumor-alone implanted mice. Hu-BLT mice were implanted with tumors and injected with NK cells, as described in FIG. 21A before spleen and BM were harvested and single cell suspensions were prepared. CD56+ NK cells were positively selected from splenocytes, and monocytes were purified from the BM cells, and co-cultured at (NK:Monocytes; 2:1 ratio) and treated with IL-2 (1000 U/mL) alone or in combination with LPS (100 ng/mL) for 7 days before the supernatants were harvested and IFN-γ secretion was determined using ELISA. One of three representative experiments is shown (FIG. 24A). Osteoclasts were generated from monocytes isolated from the BM of hu-BLT mice. Allogeneic NK cells purified from healthy individuals were treated with IL-2 (1000 U/nL) and anti-CD16 mAb (3 μg/mL) for 18 h before they were either cultured alone or in the presence of hu-BLT-OCs and sAJ2 (NK:OCs:sAJ2; 2:1:4), and the numbers of expanding NK cells were determined on days 6, 9, 12, and 15. At each day of culture, equal numbers of NK cells from each group were cultured and their cell growth was determined (n=4 to 8) (FIG. 24B). On day 15 of the culture, cells were counted, and equal numbers of NK cells were used for cytotoxicity against OSCSCs using 4-h 51Cr release assay. Lytic units 30/10⁶ cells were determined using inverse number of NK cells required to lyse 30% of the target cells×100 (n=4 to 8) (FIG. 24C). The supernatants from the NK and OC cultures as described in FIG. 24B were harvested on days 6, 9, 12, and 15, and the levels of IFN-γ secretion were determined using ELISA (n=4 to 8) (FIG. 24D). The following symbols represent the levels of statistical significance within each analysis, *** (p-value <0.001), ** (p-value 0.001-0.01), * (p-value 0.01-0.05).

FIG. 25A-25C show that the stage of differentiation in pancreatic tumors correlated with susceptibility to NK cell-mediated cytotoxicity and combination of rhTNF-α and rhIFN-γ induce differentiation and resistance of MP2 cells to NK cell-mediated cytotoxicity. Freshly isolated NK cells were left untreated or treated with IL-2 (1000 U/mL) or the combination of anti-CD16 mAb (3 μg/mL) and IL-2 (1000 U/mL) for 18 h before they were used in co-cultures with ⁵¹Cr labeled MP2, and PL12. NK cell-mediated cytotoxicity was determined using 4-hour ⁵¹Cr release assay, and the lytic units 30/10⁶ cells were determined using inverse number of NK cells required to lyse 30% of the target cells×100 (n=5 independent experiments). (FIG. 25A). Cells were left untreated or treated with rhTNF-α (20 ng/mL), rhIFN-γ (200 U/mL) or the combination of rhTNF-α (20 ng/mL) and rhIFN-7 (200 U/mL) for 24 h. Afterwards, the cells were detached and the surface expression of CD44, CD54, MHC-class I and B7H1 were assessed using staining with PE conjugated antibodies followed by flow cytometric analysis. Isotype control antibodies were used as controls (FIG. 25B) MP2 and Capan cells were treated as described in FIG. 25A, and were detached from the tissue culture plates, labeled with ⁵¹Cr and used in a standard 4-hour ⁵¹Cr release assay using IL-2 (1000 U/mL) treated NK cells. Pre-treatment of NK cells with IL-2 (1000 U/mL) were carried out for 18-24 h. Percent cytotoxicity was determined at different effector to target ratio and the lytic units 30/10⁶ cells were determined using inverse number of NK cells required to lyse 30% of the tumor cells×100 (FIG. 25C). One of the eight representative experiments is shown in the figure.

FIG. 26A-FIG. 26F show phenotypic characteristics of bone marrow, spleen, peripheral blood, pancreas in hu-BLT mice. Lack of tumor growth, metastasis and long-term survival of NSG mice after orthotopic implantation of NK-supernatant differentiated MP2 tumors in pancreas. MP2 tumors were differentiated by the NK-supernatants as described in the Example 9. Patient-derived differentiated PL12 (2×10⁶) (n=3) NK-differentiated MP2 tumors (diff-MP2) (5×10⁵) (n=3), and MP2 tumors (3×10⁵) (n=3) were implanted into the pancreas of NSG mice. The rates of survival of the mice were assessed (top right corner), and tumor growth in the pancreas as well as tumor metastasis to liver were determined after euthanasia (FIG. 18). Hu-BLT mice were generated as depicted in Figure (FIG. 26A) and described in Example 9. Reconstitution of human immune system was analyzed in PBMCs, bone marrow and splenocytes using flow cytometric analysis after staining with anti-human CD45 and anti-mouse CD45 antibodies (one of six representative experiment is shown in the figure). The percentages of human and mouse CD45+ immune cells were determined by staining with respective antibodies followed by flow cytometric analysis (FIG. 26B). PBMCs were isolated from hu-BLT mice and human donors as described in Example 9 and percentages of CD3, CD16, CD56, CD19, and CD14 within human CD45+ immune-cells were determined using antibody staining followed by flow cytometric analysis (one of six representative experiment is shown in the figure) (FIG. 26C). Hu-BLT pancreas were harvested, single cells suspension was obtained as described in Example 9 and percentages of CD3, CD19, CD8, CD4, CD16, CD56 and CD14 within human CD45+ immune-cells in pancreas were determined using antibody staining followed by flow cytometric analysis (one of six representative experiment is shown in the figure) (FIG. 26D). OCs were generated from hu-BLT bone marrow monocytes and human peripheral blood monocytes as described in Example 9. NK cells purified from hu-BLT splenocytes were pre-treated with IL-2 (1000 U/mL) and anti-CD16mAb (3 μg/mL) for 18 hours and then either cultured alone or with hu-BLT-OCs or human OCs in the presence of sAJ2 (NK: OCs: sAJ2; 2:1:4) and the numbers of expanding NK cells were counted on days 6, 10, 14, 18 and 22. At each day of culture equal numbers of NK cells from each group were cultured and cell growth determined (FIG. 26E). The supernatants from the NY cells and OCs cultures in the presence of sAJ2, as described in FIG. S2E were harvested on days 6, 10, 14, 18 and 22, the levels of IFN-γ were determined using single ELISA (FIG. 26F).

FIG. 27A-FIG. 27D show single injection of super-charged NK cells inhibited tumor growth and increased immune cells in the pancreas in hu-BLT mice. Hu-BLT mice were generated as described in Example 9. MP2 tumors were differentiated by the NK-supernatants as depicted in the figure (FIG. 27A). Hu-BLT mice were implanted with MP-2 (1×10⁶ cells) tumors in the presence and absence of super-charged NK cell injection and NK-differentiated-MP2 tumor cells (1×10⁶ cells) in the pancreas and disease progression was monitored for another 4-7 weeks (n=3 per each experimental condition). The pancreas were harvested postmortem, and percentages of CD3+ cells within human CD45+ immune cells from the pancreas were determined using antibody staining followed by flow cytometric analysis (n=3 per each experimental condition) (FIG. 27B). Hu-BLT mice were implanted with MP-2 (1×10⁶ cells) tumors in the pancreas in the presence and absence of super-charged NK cell injection (IV) and disease progression was monitored for another 4-7 weeks. Tumors were resected, and single cell cultures were prepared and cultured for 7 days, after which percentages of human CD45, CD94, CD56, NKG2D, and DNAM within the tumors were determined after staining with antibodies, followed by flow cytometric analysis (FIG. 27C). The percentages of each of CD56, CD94, NKG2D and DNAM was calculated within CD45+ cells (FIG. 27D)).

FIG. 28A-FIG. 28C show that single injection of super-charged NK-cells with/without feeding with AJ2 inhibited tumor growth due to differentiation of tumors in hu-BLT mice. Hu-BLT mice were implanted with 1×10⁶ tumor cells in the pancreas, and after 1-2 weeks mice received 1.5×10⁶ super-charged NK cells via tail vein injection, and disease progression was monitored for another 3-5 weeks. Mice were also fed AJ2 (5 billion/dose) starting 1-2 weeks before tumor implantation, and thereafter every 48 h throughout the experiment. At the end of experiment, mice were sacrificed, and pancreas/pancreatic tumor pictures were taken postmortem (n=9 per each experimental condition) (FIG. 28A). Implantation of tumor cells in the pancreas and tail vein injection of super-charged NK cells were carried out as depicted in FIG. 28A, and at the time of sacrifice mice were bled and the levels of IFN-γ in the serum were determined using multiplex array (n=3) (FIG. 28B). Highly purified healthy human NK cells were treated with IL-2 (1000 U/mL) and anti-CD16 mAb (3 μg/mL) for 18 h, after which the supernatants were collected and added to MP2 tumors in the presence/absence of anti-TNF-α (1:100) and anti-IFN-γ (1:100) for a period of 5 days. Hu-BLT mice were implanted with diff-MP2 (1×10⁶ cells) or diff-MP2 treated with monoclonal antibodies against INF-γ and TNF-α (1×10⁶ cells) and disease progression was monitored for another 4-7 weeks (n=6 per each experimental condition) (FIG. 28C).

FIG. 29A-FIG. 29D show NK cell cytotoxicity and ability to secrete IFN-γ is severely decreased in pancreatic cancer patients. PBMCs (FIG. 29A) and purified NK cells (FIG. 29B) from healthy human donors and pancreatic cancer patients were obtained and treated with IL-2 (1000 U/mL) for 18 h before they were used for cytotoxicity against OSCSCs using 4 h ⁵¹Cr release assay. The lytic units (LUs) 30/10⁶ cells were determined using inverse number of NK cells required to lyse 30% of the tumor-cells×100 (nz=3 for each experimental condition) (FIG. 29A and FIG. 29B) PBMCs (FIG. 29C) and purified NK cells (FIG. 29D) from healthy human donors and pancreatic cancer patients were treated with IL-2 (1000 U/mL) for 18 h before the supernatants were harvested and IFN-γ secretion was determined using ELISA (n=3 for each experimental condition) (FIG. 29C-FIG. 29D).

FIG. 30A-FIG. 30C show that combination of super-charged NK cells with anti-PD1 antibody injection increased IFN-γ secretion substantially by PBMCs, splenocytes and bone marrow derived immune cells and halted growth of poorly differentiated MP2 tumors in hu-BLT mice. Successfully reconstituted hu-BLT mice were orthotopically injected with 1×106 of human MP2 cells in the pancreas. One or two weeks after tumor implantation hu-BLT mice received 1.5×10⁶ super-charged NK cells via tail vein injection. Seven days later, anti-PD1 (50 μg/mice) was injected via tail vain injection. At the end of experiment, animals were sacrificed and bone marrows were harvested from hu-BLT mice and single cell suspensions were obtained as described in Example 9. Cells were treated with IL-2 (1000 U/mL), on days as specified in the figures and supernatants were harvested from cultures and IFN-γ secretion was determined using single ELISA (FIG. 30A). NK enriched cells (FIG. 30B) and CD3+ T cells (FIG. 30C) isolated from hu-BLT splenocytes were treated with IL-2 (1000 U/mL and 100 U/mL respectively), on days as specified in the figures and supernatants were harvested from cultured cells and IFN-γ secretion was determined using single ELISA (FIG. 30B and FIG. 30C). One of the two representative experiments is shown in the figure.

FIG. 31A-FIG. 31C show that OCs from pancreatic cancer patients expanded lower numbers of super-charged NK cells and generated NK cells secreted lower levels of IFN-γ when compared to healthy individuals. OCs were generated from the peripheral blood-derived monocytes of healthy human-donors and pancreatic cancer patients, as described in Example 9 and were cultured with healthy human NK cells in the presence of sonicated AJ2 (sAJ2) and the numbers of NK cells were counted on days 6, 9, 12, 15, 18 and 22. On each day of culture, equal numbers of NK cells from each group were cultured and cell growth was determined (FIG. 31A). OCs were generated from the peripheral blood-derived monocytes of healthy human-donors and pancreatic cancer patients, as described in Example 9 and were cultured with healthy human NK cells in the presence of sAJ2, and the numbers of NK cells were counted on days 6, 9, 12, 15, 18 and 22. On each day of culture, equal numbers of NK cells from each group were cultured. On day 15 of culture, NK cells were counted, and equal numbers of NK cells were used for cytotoxicity against OSCSCs using 4-hour ⁵¹Cr release assay. The lytic units (LUs) 30/10⁶ cells were determined using inverse number of NK cells required to lyse 30% of the tumor-cells×100 (FIG. 31B). Supernatants from the cultures were harvested on days 6, 9, 12, 15, 18 and 22 and the levels of IFN-γ were determined using ELISA (FIG. 31C). One of three representative experiments is shown (FIG. 31A-FIG. 31C).

FIG. 32 shows that the identical amounts of IFN-γ secreted by cancer patients NK cells in comparison to healthy donor NK cells induce lower levels of differentiation in oral cancer stem-like tumors. Supernatants containing equal amounts of IFN-γ from the healthy donor and pancreatic patients' NK cells treated with IL-2 (1000 U/mL) and anti-CD16 mAb (3 ug/mL) for 18 h were added to OSCSCs for 4 days, to induce differentiation. Allogeneic NK cells from healthy human donors were treated with IL-2 (1000 U/mL) for 18-24 h before they were used in cytotoxicity against untreated and healthy and patient NK-supernatant differentiated OSCSCs. Tumors were ⁵¹Cr labeled and used in the cytotoxicity assay, and the lytic unit (LUs) 30/10⁶ were determined using inverse number of NK cells required to.

FIG. 33A-FIG. 33E show OC-expanded NK cells from cancer patients have much lower capacity to expand, mediate cytotoxicity, and secrete IFN-γ. T cells (1×10⁶ cells/ml) from healthy individuals and cancer patients were treated with a combination of IL-2 (100 U/ml) and anti-CD3 (1 μg/ml)/CD28 mAb (3 μg/ml) for 18 hours before they were co-cultured with healthy individuals' OCs and sAJ2 at a ratio of 1:2:4 (OCs:T:sAJ2). On days 6, 9, 12, and 15, the T cells were counted using microscopy; the cumulative cell counts from day 0 to day 15 are displayed in the figure (n=7) (FIG. 33A). T cells were treated and cultured as described in FIG. 33A. The supernatants were harvested on days 6, 9, 12, and 15, and the levels of IFN-γ secretion were determined using single ELISA (n=42) (FIG. 33B). Amounts of IFN-γ secretion shown in FIG. 33B were assessed based on 1×10⁶ cells (n=42) (FIG. 33C). NK cells and T cells were treated and cultured as described in FIG. 12A and FIG. 12E respectively. The cells were counted using microscopy on days 6, 9, 12, and 15; the cumulative cell counts from day 0 to day 15 are displayed in the figure (n=10) (FIG. 33D). NK and T cells were treated and cultured as described in FIG. 12A and FIG. 12E respectively. The supernatants were then harvested on days 6, 9, 12, and 15 of the co-cultures and the amounts of IFN-γ secretion were determined using single ELISA; the cumulative levels of IFN-γ secretion from day 0 to day 15 are displayed in the figure (n=10) (FIG. 33E).

FIG. 34A-34G show that unlike those from healthy individuals, OCs from Cancer patients induced decreased cell expansion, IFN-γ secretion and cytotoxicity in allogeneic NK cells obtained from healthy individuals. NK cells (1×10⁶ cells/ml) from healthy individuals were treated with the combination of IL-2 (1000 U/ml) and anti-CD16mAb (3 μg/ml) for 18 hours before they were cultured alone or were co-cultured with either healthy individuals' OCs or cancer patients' OCs in the presence of sAJ2 at a ratio of 1:2:4 (OCs:NK:sAJ2). On days 6, 9, 12, 15, 18 and 22 of co-culture, the numbers of NK cells were counted using microscopy (n=12) (FIG. 34A). NK cells were treated and co-cultured as described in FIG. 34A. On days 6, 9, 12, 15, 18 and 22, supernatants were harvested from the co-cultures to determine IFN-γ secretion using single ELISA (n=12) (FIG. 34B). The amounts of IFN-γ secretion shown in FIG. 2B were assessed based on 1×10⁶ cells (n=12) (FIG. 34C). NK cells were treated and co-cultured as described in FIG. 34A. Cytotoxicity of days 9 and 15 cultured NK cells were determined using a standard 4-hour ⁵¹Cr release assay against OSCSCs. LU 30/10⁶ cells were determined as described in FIG. 12B (n=4) (FIG. 34D). NK cells (1×10⁶ cells/ml) from healthy individuals and cancer patients were treated with the combination of IL-2 (1000 U/ml) and anti-CD16mAb (3 μg/ml) for 18 hours before they were cultured alone, or with autologous OCs in the presence of sAJ2 at a ratio of 1:2:4 (OCs:NK:sAJ2). On days 6, 9, 12, and 16 of co-culture, the numbers of NK cells were counted using microscopy (FIG. 34E). NK cells were treated and co-cultured as described in FIG. 34E. Cytotoxicity of days 9 and 15 cultured NK cells were determined using a standard 4-hour ⁵¹Cr release assay against OSCSCs. LU 30/10⁶ cells were determined as described in FIG. 12B (n=4) (FIG. 34F). NK cells were treated and co-cultured as described in FIG. 34E. On days 6 and 12, supernatants were harvested from the co-cultures to determine IFN-γ secretion using single ELISA (FIG. 34G).

FIG. 35A-FIG. 35C show T cells from Cancer patients exhibit lower CD4+/CD8+ T cell ratio both in peripheral blood and after expansion. T cells purified from PBMCs of healthy individuals and cancer patients were analyzed for the surface expression of CD45RO, CD45RA, CD62L, CD28, CCR7, and CD127 using flow cytometry. IgG isotype control was used to assess non-specific binding. One of 12 representative experiments is shown in the figure (FIG. 35A). PBMCs isolated from the peripheral blood of healthy individuals and cancer patients were used to determine the surface expression of CD4 and CD8 using flow cytometry, and the percentages of CD4+ and CD8+ T cells were determined within CD3+ populations (n=12) (FIG. 35B), The ratio of CD4:CD8 is shown in the figure (n=12) (FIG. 35C).

FIG. 36A-FIG. 36M show that OC-expanded NK cells induced CD8+ T cell expansion whereas DC-expanded NK cells promote CD4+ T cell expansion. OCs and DCs were generated as described in Example 20. NK cells from healthy individuals (1×10⁶ cells/ml) were treated with a combination of IL-2 (1000 U/ml) and anti-CD16mAb (3 μg/ml) for 18 hours before they were co-cultured with autologous DCs or OCs in the presence of sAJ2 at 1:2:4 ratios (DCs or OCs:NK:sAJ2). The expanding cells were counted on days 8, 11, 15, and 18 using microscopy (n=30) (FIG. 36A). NK cells were co-cultured with OCs or DCs as described in FIG. 36A, and the surface expressions of CD3, CD16, and CD56 were analyzed on days 8, 11, 15, and 18 using flow cytometry. The numbers of NK cells and T cells were determined using the percentages of CD16+ and CD3+ cells, respectively, within the total cells in FIG. 36A (n=30) (FIG. 36B and FIG. 36C). NK cells were co-cultured with OCs or DCs as described in FIG. 4A and cytotoxicity of day 15 expanded cells was determined using a standard 4-hour ⁵¹Cr release assay against OSCSCs. LU 30/10⁶ cells were determined using the method described in FIG. 12B (n=12) (FIG. 36D). NK cells were co-cultured with OCs or DCs as described in FIG. 36A, and the surface expressions of CD16 were analyzed on day 15 using flow cytometry. The levels of the cytotoxicity was determined based on 1% of CD16+ NK cells (n=12) (FIG. 36E). NK cells were co-cultured with OCs or DCs as described in FIG. 36A; the supernatants were harvested on days 8, 11, 15, and 18 of the co-cultures, and the amounts of IFN-γ secretion were determined using single ELISA (n=12) (FIG. 36F). NK cells were co-cultured with OCs or DCs as described in FIG. 36A. On days 8, 11, 15, 18, 22 and 25 of the co-cultures the surface expressions of CD3+CD4+ and CD3+CD8+ T cells were determined using flow cytometry, and the percentages were used to determine the total numbers of CD3+CD4+ and CD3+CD8+ cells within the total cells (n=12) (FIG. 36G and FIG. 36H). NK cells were co-cultured with OCs or DCs as described in FIG. 36A, and the surface expressions of CD3, CD4, and CD8 were analyzed on days 8, 11, 15, and 18 using flow cytometry. Percentages of CD4+ and CD8+ T cells within the CD3+ populations are shown in this figure (n=12) (FIG. 36I). NK cells were co-cultured with OCs or DCs as described in FIG. 36A and the surface expressions of CD4, CD8, KLRG1, TIM3, and PD-1 were analyzed within CD3+ cells on day 27 of the co-cultures using flow cytometry (n=8) (FIG. 36J). T cells (1×10⁶ cells/ml) from healthy individuals were treated with a combination of IL-2 (100 U/ml) and anti-CD3 (1 μg/ml)/CD28mAb (3 μg/ml) for 18 hours before they were co-cultured with autologous DCs or OCs in the presence of sAJ2 at 1:2:4 ratios (DCs or OCs:T:sAJ2). Surface expressions of CD4, CD8, KLRG1, TIN3, and PD-1 were analyzed within CD3+ cells on day 27 of the co-culture using flow cytometry (n=8) (FIG. 36K). NK and T cells were co-cultured with OCs or DCs as described in FIG. 36A and FIG. 36K, respectively. Surface expressions of CD45RO, CD62L, CD28, CD44, CCR7, and CD127 were analyzed within CD3+ cells on day 12 of the co-culture using flow cytometry (n=8) (FIG. 36L). NK and T cells were co-cultured with OCs or DCs as described in FIG. 36A and FIG. 36K, respectively and the surface expressions of CD3, CD16, CD56, CD4, CD8, CD28, and CD62L were analyzed on day 12 of the co-culture using flow cytometry (n=8) (FIG. 36M).

FIG. 37A-FIG. 37G show that OC-induced activation increases CD8+ T cells. NK cells and T cells were purified from healthy individuals' PBMCs and the surface expressions of CD3, CD16, and CD56 on freshly isolated NK cells (left panel) and of CD3, CD4 and CD8 on freshly isolated T cells (upper right panel) were determined using flow cytometry. NK cells were treated and co-cultured with OCs and sAJ2 as described in FIG. 12A (middle right panel), and T cells were treated and co-cultured with OCs and sAJ2 as described in FIG. 33A (lower right panel). Surface expressions of CD3, CD4, and CD8 were analyzed on day 12 of the co-culture using flow cytometry (FIG. 37A). NK cells were treated and co-cultured with OCs and sAJ2 as described in FIG. 12A (left bar). Freshly purified CD8+ T cells (1×10⁶ cells/ml) from healthy individuals were treated with a combination of IL-2 (100 U/ml) and anti-CD3 (1 μg/ml)/CD28mAb (3 μg/ml) for 18 hours before they were co-cultured with OCs and sAJ2 at 1:2:4 ratios (OCs:CD4T or CD8T:sAJ2) (right bar). On days 6, 9, 12 and 15 of the co-cultures, the surface expressions of CD3+CD8+ T cells were determined using flow cytometry, and the percentages were used to determine the numbers of CD8+ T cells within the total cells. Fold expansion for each time point is shown in the figure (n=4) (FIG. 37B). Freshly purified CD8+ T cells and CD4+ T cells (1×10⁶ cells/ml) from healthy individuals were treated with a combination of IL-2 (100 U/ml) and anti-CD3 (1 μg/ml)/CD28mAb (3 μg/ml) for 18 hours before they were co-cultured with sAJ2 (T:sAJ2; 1:2) or with OCs and sAJ2 at 1:2:4 ratios (OCs:CD4T or CD8T:sAJ2). On days 6, 12, 15, and 19 of co-culture, the expanded cells were counted using microscopy (n=20) (FIG. 37C). Purified CD8+ T cells and CD4+ T cells were treated and co-cultured with sAJ2 as described in FIG. 37C. On days 6, 12, 15 and 19 of co-culture, the expanded cells were counted using microscopy. Fold expansion for each time point is shown in the figure (FIG. 37D). Freshly purified CD8+ T cells and CD4+ T cells were treated and co-cultured with sAJ2 and OCs as described in FIG. 37C. On days 6, 12, 15 and 19 of co-culture, the expanded cells were counted using microscopy. Fold expansion for each time point is shown in the figure (FIG. 37E). NK cells were treated and co-cultured with OCs as described in FIG. 12A. CD8+ T cells and CD4+ T cells were treated as described in FIG. 37C. On days 6, 9, 12 and 15 of co-culture, the expanded cells were counted using microscopy. The numbers of OC-expanded NK, OC-expanded CD4+ T and and OC-expanded CD8+ T cells were subtracted from the number of non-OC-expanded control cells and the fold expansions were determined by dividing the resulting value by the initial input cells (n=6) (FIG. 37F). NK cells were treated and co-cultured with OCs as described in FIG. 12A. CD8+ T cells and CD4+ T cells were treated as described in FIG. 37C. The supernatants were harvested from the co-cultures on days 6, 9, 12, and 15 and the secretions of IFN-γ were determined using single ELISA, and the values were adjusted based on a million lymphocytes (n=3) (FIG. 37G).

FIG. 38A-FIG. 38B show that NK cells preferentially lyse CD4+ T cells and not CD8+ T cells. Freshly purified CD4+ T and CD8+ T cells from healthy individuals were left untreated, treated with IL-2 (100 U/ml), or treated with a combination of IL-2 (100 U/ml) and anti-CD³ (1 μg/ml)/CD28mAb (3 pig/ml) for 18 hours. NK cells which were treated and co-cultured with OCs as described in FIG. 12A, were used to determine cytotoxicity against CD4+ and CD8+ T cells using a TVA dye assay. LU 30/10⁷ cells were determined using the inverse of the number of NK cells required to lyse 30% of target cells×1000 (n=4) (FIG. 38A). NK cells were left untreated or treated with IL-2 (1000 U/ml) for 18 hours, or expanded with OCs as described in FIG. 12A, and used to determine cytotoxicity against IL-2 (100 U/ml) and anti-CD3 (1 μg/ml)/CD28mAb (3 μg/ml) Treated CD4+ and CD8+ T cells using a TVA dye assay. LU 30/10? cells were determined as described in FIG. 38A. One of several representative experiments is shown in the figures (FIG. 38B).

FIG. 39A-FIG. 39B show that OC-mediated activation of NK cells induced lower secretion of IFN-γ from cancer patients' NK cells in comparison to healthy individuals. Purified NK cells (1×10⁶ cells/ml) from healthy individuals and cancer patients were treated with a combination of IL-2 (1000 U/ml) and anti-CD16 mAb (3 ug/ml) for 18 hours before they were treated with sAJ2 at a ratio of 1:2 (NK:sAJ2). The supernatants were then harvested from the co-cultures on days 6, 9, 12, and 15, and IFN-γ secretions were determined using single ELISA (n=8) (FIG. 39A). Monocytes were purified from healthy individuals' PBMCs and were then cultured in alpha-MEM media supplemented with M-CSF (25 ng/ml) and RANKL (25 ng/ml) for 21 days to generate OCs. Purified NK cells (1×10⁶ cells/ml) from the healthy individuals and cancer patients were treated with a combination of IL-2 (1000 U/ml) and anti-CD16 mAb (3 ug/ml) for 18 hours before they were co-cultured with sAJ2 and OCs at a ratio of 1:2:4 (OCs:NK:sAJ2). The supernatants were then harvested from the co-cultures on days 6, 9, 12, and 15, and IFN-γ secretions were determined using single ELISA (n=8) (FIG. 39B).

FIG. 40A-FIG. 40F show decreased expansion and function of T cells from cancer patients with and without OC-mediated activation when compared to those from healthy individuals. OCs were generated as described in FIG. S2B. Purified T cells (1×10⁶ cells/ml) from healthy individuals and cancer patients were treated with a combination of IL-2 (100 U/ml) and anti-CD3 (1 μg/ml) and anti-CD28 for 18 hours before they were treated with sAJ2 with and without OCs at a ratio of 1:2:4 (OCs:T:sAJ2). Cells were counted using microscopy on days 6, 9, 12, and 15 of the co-cultures and the cumulative cell counts of lymphocytes from day 0-day 15 were determined (n=4) (FIG. 40A). Purified T cells from the healthy individuals and cancer patients were treated and cultured with OCs as described in FIG. 40A. The supernatants were harvested on days 6, 9, 12, and 15 of the co-cultures, and levels of IFN-γ were measured using single ELISA; the cumulative amounts of IFN-γ detected from day 0-day 15 is shown in the figure (n=4) (FIG. 40B) and the corresponding amount were adjusted based on 1 million cell counts (n=4) (FIG. 40C). Purified T cells from healthy individuals and cancer patients were treated with a combination of IL-2 (100 U/ml) and anti-CD3 (1 μg/ml) and anti-CD28 for 18 hours before they were treated with sAJ2 at a ratio of 1:2 (T:sAJ2). The supernatants were then harvested on days 6, 9, 12, and 15 of the co-cultures, and levels of IFN-γ were determined using single ELISA (n=4) (FIG. 40D). Purified T cells from the healthy individuals and cancer patients were treated and cultured with OCs as described in FIG. 40A. The supernatants were harvested on days 6, 9, 12, and 15 of the co-cultures, and IFN-γ secretions were determined using single ELISA (n=4) (FIG. 40E). Freshly purified NK cells from the healthy individuals were treated and cultured as described in FIG. 39A and FIG. 39B. Purified T cells from the healthy individuals were treated and cultured as described in FIG. 40A. Cells were then counted using microscopy on days 6, 9, 12, and 15 of the co-cultures and the cumulative lymphocyte counts from day 0-day 15 were determined (n=4) (FIG. 40F).

FIG. 41A-FIG. 41C shows OC-mediated activation induced higher secretion of cytokines and chemokines from NK cells when compared to T cells. Freshly purified NK cells from the healthy individuals were treated and co-cultured as described in FIG. 39B. On day 12 of the co-culture; NK and NK-expanded CD8+ T cells were isolated from the expanded NK cells using the corresponding isolation kits. NK cells were treated with a combination of IL-2 (1000 U/ml) and anti-CD16 mAb (3 ug/ml) and, CD8+ T cells were treated with IL-2 (100 U/ml) and anti-CD3 (1 μg/ml)/CD28 mAb (3 ug/ml) for 18 hours. The supernatants were then harvested and were used to determine the levels of cytokines, chemokines, and growth factors using multiplex array kits. The amounts of all tested factors were adjusted based on 1 million cell counts and, the ratios of secretion between NK and CD8+ T cells (NK/CD8+ T cells) were determined and fold increase in the secreted levels for NK cells were determined (FIG. 41A). Freshly purified NK cells from the healthy individuals were treated and co-cultured as described in FIG. 39B. Freshly purified T cells from the healthy individuals were treated and co-cultured with OCs as described in FIG. 40A. The supernatants were then harvested on day 6 of co-culture, and the levels of cytokines, chemokines, and growth factors were measured using multiplex array kits. Ratios of secretion between NK and T cells (NK/T cells) were determined and fold increase in the secreted levels for NK cells were determined. (FIG. 41B). The secreted levels shown in FIG. 41B were adjusted based on 1 million cell counts and, the ratios of secretion between NK and T cells (NK/T cells) were determined and fold increase in the secreted levels for NK cells were determined (FIG. 41C). Freshly purified NK cells from the healthy individuals were treated and co-cultured with OCs as described in FIG. 39B. In a separate culture, freshly isolated CD8+ T cells purified from healthy individuals were treated with IL-2 (100 U/ml) and anti-CD3 (1 μg/ml)/CD28 mAb (3 ug/ml) for 18 hours before they were cultured with OCs at a ratio of 1:2:4 (OCs:CD8+ T:sAJ2). On day 12, CD8+ T cells were isolated from OC-expanded NK cells. CD8+ T cells isolated from OC-expanded NK cells and those from OC-expanded CD8+ T cells were further treated with IL-2 (100 U/ml) and anti-CD3 (1 μg/ml)/CD28 mAb (3 ug/ml) and after 18 hours of incubation, the supernatants were harvested from both CD8+ T cells cultures, and the levels of cytokines, chemokines, and growth factors were measured using multiplex array kits. Ratios of secretion between CD8+ T cells isolated from OC-expanded NK cells and OC-expanded CD8+ T cells were determined and fold increase in the secreted levels for CD8+ T cells isolated from OC-expanded NK cells were determined (FIG. 41D). One of three representative experiments is shown in FIG. 41.

FIG. 42 shows that OCs from Cancer patients had lower ability to expand autologous CD8+ T cells both in NK cells and T cells co-cultures in comparison to those from healthy individuals. Freshly purified NK cells (1×10⁶ cells/ml) from healthy individuals and cancer patients were treated with the combination of IL-2 (1000 U/ml) and anti-CD16mAb (3 μg/ml) for 18 hours. NK cells from healthy individuals and cancer patients were co-cultured with their respective autologous OCs in the presence of sAJ2 at a ratio of 1:2:4 (OCs:NK:sAJ2). Purified T cells (1×10⁶ cells/ml) from healthy individuals and cancer patients were treated with the combination of IL-2 (100 U/ml) and anti-CD³ (1 μg/ml)/CD28mAb (3 μg/ml) for 18 hours and then co-cultured with their respective autologous OCs. On day 9 of co-culture, surface expression of CD4 and CD8 were analyzed using flow cytometry, and the percentages of CD4+ and CD8+ T cells within CD3+ T cells were determined.

### DETAILED DESCRIPTION OF THE INVENTION

The present invention relates, in part, to methods that determine the expansion potential and function of NK cells, and/or the suitability of NK cells for NK cell-based immunotherapies. Such methods can be employed for treating diseases such as cancer. However, the utility of the methods is not limited to the treatment of cancer, as NK cell-based therapies generally strengthen the immune system of a subject and thus can be useful to bolster a subject's immune system in any situation where that might be beneficial.

Various standards may be used for evaluating the cytotoxic function of test NK cells as disclosed herein. For example, the cytotoxic function of the test NK cells may be measured by co-incubating the test NK cells with the target cells, e.g., cancer stem cells (direct killing); or by co-incubating the test NK cells, the target cells, and antibodies to the cell surface marker expressed on the target cells (ADCC activity). In such assays, the cytotoxic function of NK cells may be measured e.g., as percentage of target cells killed by the test NK cells in a given time frame, compared to the percentage of target cells killed by non-NK cells that do not have cytotoxic function. Alternatively, the percentage of target cells killed by the test NK cells may be compared to the percentage of target cells killed without the NK cells (e.g., natural death) or to the percentage of target cells killed by reference NK cells of known activity (e.g., cell line, NK cells of known or defined function and expansion potential). Similarly, the percentage of target cells killed by the test NK cells may be compared to a predetermined percentage, e.g., a percentage representative of a percentage of cells typically killed by active NK cells in vitro, ex vivo, or in vivo. For example, the predetermined percentage may be derived from a pool of NK cells from multiple subjects, either diseased or healthy subjects. In some cases, the predetermined percentage may be adjusted based on the severity of the patient's cancer and/or the number of e.g., cancer cells required to kill to treat cancer in a given subject. In some embodiments, the percentage of cells killed by the test NK cells is qualitatively compared (e.g., visualization under the microscope, MFI plots from FACS analysis) to any of the controls or references mentioned above.

Alternatively, the cytotoxic function of the test NK cells may be represented as the number of test NK cells required to kill a certain percentage of target cells. This number can then be compared to a corresponding reference or control number determined for any of the references or controls outlined above. Similarly, any other measure of the cytotoxic function of the test NK cells may be compared to a corresponding control or reference value that permits the comparative evaluation of the cytotoxic function of the test NK cells quantitatively or qualitatively.

Similarly, various standards may be used for evaluating the amount of IFN-g produced by test NK cells as disclosed herein. For example, the amount of IFN-g produced by the test NK cells may be measured and compared with the amount of IFN-g produced by non-NK cells or by reference NK cells (e.g., cell line, NK cells of known or defined function and expansion potential). The amount of IFN-g produced by NK cells may be represented as concentration. In some such embodiments, the concentration is compared to the concentration of IFN-g in a subject (e.g., serum, whole blood, tumor). The amount or concentration of IFN-g produced by the test NK cells may be compared to a predetermined value such as a value representative of a sample of multiple subjects of known condition (e.g., diseased or healthy).

Various standards may be also used for evaluating the ability of test NK cells' IFN-g to induce differentiation of tumor cells as disclosed herein. For example, where the differentiated tumor cells grow slowly or their cell divisions are inhibited, the IFN-g's ability to decrease and/or inhibit tumor growth and/or tumor cell division is monitored. The measured growth and/or cell division of tumor cells co-incubated with the test NK cells' IFN-g may be compared to the growth and/or cell division of tumor cells without IFN-g, to the growth and/or cell division of tumor cells with reference IFN-g of known activity, or to the growth and/or cell division of already-differentiated cells. Similarly, the measured growth and/or cell division of tumor cells co-incubated with test NK cells' IFN-g may be compared to the growth and/or cell division of suitable undifferentiated cells (e.g., cancer cell line, cancer stem cells).

The ability of test NK cells' IFN-g to induce differentiation of tumor cells can be evaluated by looking at changes in cell markers associated with cell differentiation. For example, the level of one or more different cell surface markers (e.g., CD44, CD54, MHC class I, PD-L1) indicative of various differentiation states may be measured after incubation or treatment with the test NK cells' IFN-g. The measured level(s) of markers on cells treated with the test NK cells' IFN-g can then be compared to level(s) of the markers on cells not treated with IFN-g, differentiated cells (e.g., normal primary cells, non-transformed cells), or undifferentiated cells (e.g., cancer cell line, cancer stem cells). Alternatively, the level(s) of such markers before and after treatment with the test NK cells' IFN-g can be measured and compared to assess the effects of the IFN-g on those cells.

Various standards may be used for evaluating the ability of test NK cells' IFN-g to induce differentiation of tumor cells as disclosed herein. For example, where the differentiated tumor cells are resistant to NK cell-mediated killing, the tumor cells incubated with the test NK cells' IFN-g may be tested for the degree of killing by reference NK cells (e.g., those known to have cytotoxic function). In measuring the reference NK cell-mediated cytotoxic killing of the tumor cells that are differentiated by the test NK cells' IFN-g, any one of the comparators disclosed above for testing the NK cell cytotoxic function may be used.

In certain embodiments, other methods for evaluating the intrinsic activity of IFN-g can similarly be used to compare the activity of test NK cells' IFN-g with the activity of normal IFN-g, or to assess the activity of test NK cells' IFN-g on an absolute scale and compare to suitable reference or control values.

Various standards may be used for evaluating the ability of test NK cells to be expanded by osteoclast cells. In some embodiments, the number of NK cells after co-incubation with osteoclast cells is compared to the number of NK cells before co-incubation with osteoclasts, to the number of NK cells of a control cell population cultured without co-incubation with osteoclasts, or to a predetermined value such as a value representative of a sample of reference (e.g., normal) NK cells expanded by osteoclasts. These osteoclasts may or may not be autologous to the NK cells.

Various standards may be used for evaluating the ability of the sample NK cells to expand CD8+ T cells. The number of CD8+ T cells after NK cell-mediated expansion may be compared to the number of CD8+ T cells before NK cell-mediated expansion, to the number of CD8+ T cells without expansion by the NK cells, or to a predetermined value such as a value representative of a sample of CD8+ T cells expanded by a reference (e.g., normal) NK cells. Alternatively, the predetermined value may represent a value representative of the expansion potential of NK cells from multiple subjects of known condition (e.g., afflicted with cancer or healthy). In preferred embodiments, the number of CD8+ T cells expanded by NK cells is determined relative to the number of CD4+ T cells expanded by the same NK cells. The ratio of CD8+ T cells to CD4+ T cells may be compared to any of the controls and/or reference standards mentioned above.

Similarly, various standards may be used for evaluating the function of CD16 receptors on the test NK cells. For example, the amount of IFN-g secreted by the NK cells in response to treatment with CD16 may be determined and compared to any of the controls and/or reference standards mentioned above, especially those related to the measurements of IFN-g. The ability of the test NK cells to mediate ADCC function against differentiated tumors in response to CD16 may alternatively or additionally be measured and used as a basis for comparison. The measured ADCC function may be compared to any of the controls and/or reference standards mentioned above, especially those related to measuring the NK cells' cytotoxic function.

Various standards may be used for evaluating the amount of CD16 receptor on the NK cells. For example, the measured amount may be compared to the amount of CD16 receptor on normal NK cells, e.g., NK cells from a healthy subject and/or a subject free of cancer. Alternatively, the measured amount may be compared to the amount of CD16 receptor on reference NK cells (e.g., cell line).

Various standards may be used for evaluating the amount of CD44, CD54, MHC class I, PD-L1 (B7H1), MICA, and/or MICB. The amount measured in cancer cells may be compared to the amount of the same marker on any undifferentiated cancer cells (e.g., cancer cell line, cancer stem cells) or differentiated cancer cells (e.g., normal primary cells, non-transformed dysplastic cells). Similarly, the markers described above may also be characterized on tumor tissues and/or dissociated cells thereof.

It will be understood that for the methods described herein for assessing NK cells using multiple assays, it is not necessary that all of the assays be conducted on the exact same cells. NK cells may be assessed by testing a first sample of the NK cells in a first assay, a second sample of the cells in a second assay, etc., provided that each sample is effectively random and representative of the NK cells as a group. For methods of assessing the NK cells of a subject, it is preferred that the NK cells be obtained in a single sampling of the patient, but NK cells can be obtained in multiple samplings, possibly even at different times, so long as the NK cells at those different times are still reasonably representative of the subject's NK cells.

Accordingly, various samples may be used for the assays described herein. In some embodiments, the patient tumor tissues are obtained and the characteristics (e.g., differentiation state) of the tumor tissues may be analyzed (e.g., via immunohistochemistry). In addition to, or alternatively, the tumor cells and the infiltrating immune cells may be dissociated from the tumor tissues (e.g., mechanically or chemically) and analyzed using the assays described herein. The analysis of tumor tissues and/or dissociated cells may be compared with the analysis of the patient's infiltrating immune cells (e.g., NK cells), which allows important in vivo determination of the state of immune function against tumor cells/tissues and/or the state of tumor cells, e.g., the differentiation stage of tumor cells. For example, high infiltration of tumor cells with immune cells may indicate a high differentiation stage of tumors corresponding to smaller tumor sizes. Thus, these tests provide a valuable prognostic tool as well as a guide for devising treatment strategies for the cancer patients.

Numerous applications of these assays are provided herein. These assays may be used to assess the state of NK cells of healthy individuals as well as those of diseased patients (e.g., patients afflicted with cancer) whose NK cell function and/or expansion potentials may be compromised. These assays may also be used to assess whether a patient's autologous NK cells are suitable for immunotherapy. Such assessment may be made with or without additional expansion and/or activation of the NK cells. These assays may be used to determine whether NK cells allogeneic to the patients should be used for immunotherapy. These assays may be used to further determine whether such allogeneic NK cells are suitable for the immunotherapy.

The assays provided herein may be used to select appropriate therapeutic regimens for a patient. For example, if a patient's NK cells are determined to have suitable cytotoxicity, but exhibit substandard levels of IFN-γ secretion and/or IFN-γ tumor differentiation potency, the patient may be selected to receive therapy with IL-2 (preferably at low doses) and/or probiotic bacteria as described in greater detail herein in order to increase the levels of IFN-γ secretion in the patient's NK cells. Such patients may also benefit from infusion of NK cells (autologous or allogeneic) that have been expanded and/or activated, e.g., by one or more of the methods described herein.

Analogously, if the patient's NK cells produce a standard amount of IFN-γ but exhibit substandard expansion potential, cytotoxicity, or ability to expand CD8+ T cells, then the patient may be selected to receive a therapeutic regimen of IL-2, IL-15, and/or IL-21. Alternatively or additionally, the patient may be selected to receive therapy with IL-2 (preferably at low doses) and/or probiotic bacteria, e.g., as described in greater detail herein, in order to improve the cytotoxic function of the patient's NK cells. In patients with split anergized NK cells, in which the cytotoxicity is deficient but the IFN-g secretion is maintained, IFN-γ can promote differentiation of tumors to make them susceptible to chemotherapy. Accordingly, such patients may be further selected to receive treatment with chemotherapy and/or radiotherapy.

If multiple NK cell functions (e.g., cytotoxicity, expansion potential, IFN-γ secretion, IFN-γ tumor differentiation potency, ability to expand CD8+ T cells) of a patient's NK cells are determined to be substandard, then the patient may be selected to receive infusions, e.g., repeated infusions, of NK cells (autologous or allogeneic), e.g., NK cells that have been expanded and/or activated by one or more of the methods described herein such that they meet the standards for all or almost all of the NK cell functions assessed in the assays described herein. Such patients may also be selected to receive treatment with IL-15, IL-2 (e.g., low doses), and/or probiotic bacteria as described herein.

In certain preferred embodiments, the methods may further comprise administering to the patients one or more of the treatments they have been selected to receive.

Various criteria may be used to determine whether NK cells are standard or substandard in function and/or expansion potential. In certain preferred embodiments, NK cells may be considered substandard if the efficiency at which the NK cells kill cancer cells and/or cancer stem cells (e.g., direct killing and/or ADCC-dependent killing) is less than 25% of the efficiency at which healthy NK cells kill cancer cells and/or cancer stem cells. In some embodiments, the number of NK cells needed to mediate killing of one cancer cell may be determined. For example, in order to kill one cancer cell (direct killing and/or ADCC-dependent killing), at least two substandard NK cells may be needed. By contrast, in order to kill one cancer cell, only about 0.25 to about 0.5 standard or healthy NK cells is typically needed, i.e., one healthy NK cells may be able to kill more than one cancer cells or cancer stem cells, such as two, three, or even four cancer cells or cancer stem cells.

In some embodiments, NK cells may be considered substandard if the amount of IFN-γ produced by the NK cells when treated with IL-2 is less than about 33% of the amount of IFN-γ produced by standard or healthy NK cells when treated with IL-2. In certain embodiments, the NK cells may be considered substandard if the amount of IFN-γ produced by each million NK cells when treated with IL-2 is less than about 100 picograms (pg), as measured by, e.g., ELISA. By contrast, each million of standard or healthy NK cells may produce about 3-fold more IFN-γ (about 300 picograms). Analogously, NK cells may be considered substandard if NK cells produce less than about 30% of the amount of IFN-γ produced by healthy NK cells at a single cell level. For example, a substandard NK cell may produce less than about 20-30 spots as measured by e.g., ELISPOT. This is in contrast to healthy NK cells that may produce greater than 100 spots, which may even be too numerous to accurately count.

In certain aspects, NK cells may be considered substandard if the IFN-γ produced by the NK cells are not able to induce differentiation of tumor cells. For example, NK cells may be substandard if the IFN-γ produced by the NK cells does not decrease or inhibit tumor growth and/or tumor cell division by at least 50%. In certain embodiments, NK cells may be substandard if the IFN-γ produced by the NK cells does not decrease the expression level of CD44 and/or increase the expression level of at least one of CD54, MHC class I, and PD-L1 as compared to the expression level of the same markers in the control by at least 3 fold. In some embodiments, NK cells may be substandard if the IFN-γ produced by the NK cells increases resistance of the tumor cells to the NK-cell-mediated cytotoxicity as compared to the resistance in the control by less than about 60-70%.

In certain aspects, NK cells are considered in need of activation by one or more methods described in detail herein. In some embodiments, NK cells as considered substandard by any criterion outlined above need to be activated further. In some embodiments, similar but variations of the criteria set forth above may be considered. For example, NK cells may be considered in need of activation, if NK cells produce at a single cell level less than about 20-40 spots as measured by, e.g., ELISPOT. In certain embodiments, NK cells may be considered in need of activation if the IFN-γ produced by the NK cells does not decrease or inhibit tumor growth and/or tumor cell division by at least 50%. Furthermore, NK cells may be considered in need of activation if the IFN-γ produced by the NK cells decreases an expression level of CD44 and/or increases an expression level of at least one of CD54, MHC class I, and PD-L1 as compared to the expression level of the same markers in the control by less than about 2-3 fold. In addition, NK cells may be considered in need of activation if the IFN-γ produced by the NK cells increases resistance of the tumor cells to the NK-cell-mediated cytotoxicity as compared to the resistance in the control by less than about 50-70%. After activation, the NK cells can be re-tested according to the originally substandard criterion or criteria, or can be retested in the original panel of assays.

In some embodiments, the ability of the NK cells to be expanded by the osteoclast cells may be determined. For example, NK cells may be considered substandard if the NK cells are not expanded by osteoclast cells to at least about 17-21 population doubling within 4 weeks.

In certain embodiments, the ability of NK cells to expand CD8+ T cells may be assessed. For example, NK cells may be considered substandard if the NK cells do not expand CD8+ T cells to at least 10 fold.

In certain embodiments, the amount and/or function of CD16 receptors on NK cells is assessed. NK cells may be considered substandard if the NK cells show at least 20% decrease in the level of CD16 expression (as measured by e.g., flow cytometry (MFI), Western blot, PCR to detect mRNA/cDNA).

### VIII. NK Cell Activation

If NK cells are deemed inadequately active or inactive by the criteria set forth by the instant assays, any of a number of methods can be used to activate the NK cells. Suitable methods are known in the art and/or are disclosed herein. Certain preferred embodiments of activating the NK cells include those disclosed in International Patent Applications WO 2018/112366 and WO 2018/152340, hereby incorporated herein by reference.

The NK cells may be activated by contacting, e.g., in vitro, ex vivo, or in vivo, the NK cells with monocytes expressing an amount of CD16 sufficient to activate the NK cells. In some such embodiments, the monocytes comprise an exogenous DNA that induces expression of CD16. Alternatively, the NK cells may be activated by contacting with at least one of IL-2, CD16, anti-CD16 antibody, anti-CD3 antibody, anti-CD28 antibody, and a composition comprising at least one bacterial strain, e.g., a probiotic composition, preferably comprising sAJ2 bacteria. A number of suitable probiotic compositions are disclosed herein and in International Patent Application, WO18/112366.

After activation, the NK cells can be re-tested according to the originally substandard criterion or criteria, or can be retested in the original panel of assays. If the NK cells pass retesting, they can be administered to a patient or otherwise treated as though they had been deemed to meet the standard(s) in the original set of assays.

### I. Definitions

The articles “a” and “an” are used herein to refer to one or to more than one (i.e. to at least one) of the grammatical object of the article. By way of example, “an” element means one element or more than one element.

The term “administering” is intended to include routes of administration which allow a therapeutic to perform its intended function. Examples of routes of administration include injection (subcutaneous, intravenous, parenterally, intraperitoneally, intrathecal, etc.), oral, inhalation, and transdermal routes. The injection can be a bolus injection or continuous infusion. The therapeutic may be administered alone, or in conjunction with a pharmaceutically acceptable carrier. Examples of routes of administration further include transplantation or grafting of cells into the body that may or may not be preceded by a surgical opening of the body. The immune boosting agents, e.g., NK cells, CD8+ T cells, of the invention are preferably administered to subjects in a biologically compatible form suitable for pharmaceutical administration in vivo, to enhance immune cell mediated immune responses. By “biologically compatible form suitable for administration in vivo” is meant a form to be administered in which any toxic effects are outweighed by the therapeutic effects.

The term “activating” or “activation” refers to an enhancement of the function of a target. For example, the instant disclosure provides a method of activating a NK cell in vitro, ex vivo, and/or in vivo. In the instant disclosure, the activation of a cell refers to an enhancement of the function of such cell, including at least an enhancement of activity and/or at least one cellular function (e.g., cytotoxicity, cell division and/or growth rate, etc.). In some embodiments, an agent used herein activates at least one cell, such as an NK cell(s). In some embodiments, an agent used herein activates at least one function of a cell, such as an NK cell(s).

The term “NK cell function(s)” refers to any function of NK cells, such as cytotoxicity and/or cytokine/chemokine production/secretion activities, including secretion of IFN-γ.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. In some embodiments, such cells exhibit such characteristics in part or in full due to at least one genetic mutations. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is oral cancer, oral squamous carcinoma, breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “control” refers to any suitable reference standard, such as a normal patient, cultured primary cells/tissues isolated from a subject such as a normal subject, adjacent normal cells/tissues obtained from the same organ or body location of the patient, a tissue or cell sample isolated from a normal subject, or a primary cells/tissues obtained from a depository. In other preferred embodiments, the control may comprise an expression level, numbers of a certain cell type (e.g., NK cells or monocytes), and/or a cellular function of a certain cell type for a set of subject, such as a normal or healthy subject. In some embodiments, a control refers to a sample lacking the test agent, e.g., IFN-g.

The term “control” also refers to any reference standard suitable to provide a comparison to the expression products in the test sample. In one embodiment, the control comprises obtaining a “control sample” from which expression product levels, e.g., biomarkers on NK cells, tumor cells, monocytes, are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In some embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention.

In some embodiments, the amount of proteins or nucleic acids may be determined within a sample relative to, or as a ratio of, the amount of proteins or nucleic acids of another gene in the same sample. In some embodiments, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the present invention are not limited to use of a specific cut-point in comparing the level of expression product in the test sample to the control.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “cytokine” refers to a broad and loose category of small proteins (˜5-20 kDa) that are important in cell signaling. Their release has an effect on the behaviour of cells around them. cytokines are involved in autocrine signaling, paracrine signaling and endocrine signaling as immunomodulating agents. Cytokines include chemokines, interferons, interleukins, lymphokines, and tumour necrosis factors, and may additionally include hormones or growth factors in the instant disclosure. Cytokines are produced by a broad range of cells, including immune cells like macrophages, B lymphocytes, T lymphocytes and mast cells, as well as endothelial cells, fibroblasts, and various stromal cells. Preferred cytokines are exemplified in the specification and the Figures of the instant disclosure.

The term “cytokine/chemokine activity,” includes the ability of a cytokine or a chemokine to modulate at least on of cellular functions. Generally, cytokines or chemokines modulate the balance between humoral and cell-based immune responses, and they regulate the maturation, growth, and responsiveness of particular cell populations. Thus, the term “cytokine/chemokine activity” includes the ability of a cytokine or chemokine to bind its natural cellular receptor(s), the ability to modulate cellular signals, and the ability to modulate the immune response.

The term “immune response” includes NK-mediated, T cell mediated, and/or B cell mediated immune responses. Exemplary immune responses include T cell responses, e.g., cytokine production and cellular cytotoxicity. In addition, the term immune response includes immune responses that are indirectly affected by NK cell or T cell activation, e.g., antibody production (humoral responses) and activation of cytokine responsive cells, e.g., macrophages.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. In some embodiments, immunotherapy comprises administration of immune cells. In some embodiments, immunotherapy comprises administration of NK cells and/or CD8+ T cells to a subject. The NK cells and/or CD8+ T cells may be autologous or allogeneic to the subject. The NK cells and/or CD8+ T cells may be expanded, modified, and/or activated in vitro, ex vivo, or in vivo.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “inhibit” includes the reduce, decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “subject” refers to any healthy animal, mammal or human, or any animal, mammal or human afflicted with a cancer, e.g., brain metastasis, oral cancer, lung, ovarian, pancreatic, liver, breast, prostate, colon carcinomas, melanoma, multiple myeloma, and the like. The term “subject” is interchangeable with “patient.”

The term “therapeutic effect” refers to a local or systemic effect in animals, particularly mammals, and more particularly humans, caused by a pharmacologically active substance. The phrase “therapeutically-effective amount” means that amount of such a substance that produces some desired local or systemic effect at a reasonable benefit/risk ratio applicable to any treatment.

The term “amount” or “level” refers to a copy number of a nucleic acid, and/or the amount or level of a protein. The amount or level of a nucleic acid or a protein may be determined using any methods known in the art.

As used herein, the amount of a biomarker or activity (e.g., CD16, CD44, CD54, MHC class I, PD-L1 (B7H1), MICA, MICB, IFN-g, cytotoxic function, number of expanded T cells, number of NK cells expanded by osteoclasts, etc.) in a sample is “significantly” higher or lower than the normal/control amount of the biomarker or activity, if the amount is greater or less, respectively, than the normalcontrol level by an amount greater than the standard error of the assay employed to assess amount, and preferably at least 10%, 20%, 30%, 40%, 50%, 60%, 70%, 80%, 90%, 100%, 150%, 200%, 300%, 350%, 400%, 500%, 600%, 700%, 800%, 900%, 1000% or than that amount. Such “significance” can be assessed from any desired or known point of comparison, such as a particular post-treatment versus pre-treatment biomarker measurement ratio, e.g., differentiation of tumor cells by treatment of IFN-g, (e.g., 1-fold, 1.1-fold, 1.2-fold, 1.3-fold, 1.4-fold, 1.5-fold, and the like). Alternately, the amount of the biomarker or activity in the sample can be considered “significantly” higher or lower than the normal amount if the amount is at least about two, three, four, or five times, higher or lower, respectively, than the normal amount of the biomarker or activity. Such “significance” can also be applied to any other measured parameter described herein, such as for expression, cytotoxicity, cell growth, and the like.

The instant inventions use antibodies in assays including the antibody-dependent cellular cytotoxicity assays and for detecting the biomarkers. Antibodies may be polyclonal or monoclonal; xenogeneic, allogeneic, or syngeneic; or modified forms thereof (e.g., humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

### NK Cells

Natural killer cells or NK cells are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

NK cells are negatively regulated by major histocompatibility complex (MHC) class I-specific inhibitory receptors (Karre et al., 1986; Ohlen et al, 1989). These specific receptors bind to polymorphic determinants of MHC class I molecules or HLA present on other cells and inhibit NK cell lysis. In humans, certain members of a family of receptors termed killer Ig-like receptors (KIRs) recognize groups of HLA class I alleles.

KIRs are a large family of receptors present on certain subsets of lymphocytes, including NK cells. The nomenclature for KIRs is based upon the number of extracellular domains (KIR2D or KIR3D) and whether the cytoplasmic tail is either long (KIR2DL or KIR3DL) or short (KIR2DS or KIR3DS). Within humans, the presence or absence of a given KIR is variable from one NK cell to another within the NK population present in a single individual. Within the human population there is also a relatively high level of polymorphism of the KIR molecules, with certain KIR molecules being present in some, but not all individuals. Certain KIR gene products cause stimulation of lymphocyte activity when bound to an appropriate ligand. The confirmed stimulatory KIRs all have a short cytoplasmic tail with a charged transmembrane residue that associates with an adapter molecule having an immunostimulatory motif (ITAM). Other KIR gene products are inhibitory in nature.

Natural killer cells constitute about 10% of peripheral blood mononuclear cells in human blood, and are identified by their lack of surface expression of CD3 and expressions of CD16 and CD56. NK cells mediate both direct and antibody-dependent cellular cytotoxicity (ADCC) against tumor cells and virally infected cells. They can recognize these cells without prior sensitization. NK cells mediate direct cytotoxicity by releasing pre-formed granules known as perforn and granzyme B, which can induce necrosis and apoptosis. When NK cell recognize its target cells and forms the lytic immunological synapse, the secretory lysosome polarizes towards the synapse and move into close proximity to the plasma membrane. Perforin, a membrane-disrupting protein, facilitates delivery of the Granzyme, a serine protease, which cleaves a variety of targets, such as caspases, resulting in cell death. NK cells can also mediate direct cytotoxicity via death receptors on the target cells through surface expression of their ligands such as Fas Ligand, Trail and TNF-alpha. Fas (CD95/APO-1/TNFRSF6), a cell surface protein that belongs to the tumor necrosis factor receptor family, can mediate apoptosis when bound to its natural ligand, CD95L (CD178/TNFSF6) or stimulated with agonistic antibodies. NK cells can mediate antibody dependent cellular cytotoxicity (ADCC) against tumors and regulate the function of other cells through the secretion of cytokines and chemokines.

Two major subsets of NK cells have been identified, one with the surface expression of CD16⁻⁺⁺CD56⁺, which is the predominant subset in the circulating blood with high cytotoxicity, whereas the other is CD16⁻CD56⁺⁺⁻ subset residing in the mucosa known as the regulatory subset. Our Laboratory has established four different stages of NK cell maturation. Stage one NK cells are CD16⁻⁺⁺, CD56⁺, CD69⁻, and CD107a⁺ found to select and kill cancer stem-like cells/undifferentiated tumors. Upon IL-2 activation and CD16 receptor triggering NK cells express CD16^{+/-}CD56⁺⁺CD69⁺CD107a⁺ and increase secretion of IFN-γ and TNF-α while exhibiting decreased cytotoxicity. This is the second stage and NK cells in this stage are known as split-anergized NK cells. Without further activation NK cells move towards stage three where they become non-functional and lose their cytotoxicity and cytokine secretion ability. Finally, NK cells may undergo apoptosis giving rise to stage 4.

### Probiotic Bacteria

In some embodiments, the methods disclosed herein use a composition comprising at least one probiotic bacterial strain capable of regulating NK cell function. Such probiotic bacteria induce significant split anergy in activated NK cells, leading to a significant induction of IFN-γ and TNF-α. In addition, such probiotic bacteria induce significant expansion of NK cells. Exemplary probiotic bacteria useful for this purpose are disclosed in International Patent Application WO18/112366, hereby incorporated herein by reference, in particular for the probiotic bacteria it discloses.

Many commercial probiotics are available, having various effects of reducing gastrointestinal discomfort or strengthening of the immune system. Preferred probiotic bacteria species for use in the compositions and methods described herein include those commercially available strains of probiotic bacteria (such as AJ2 bacteria), especially those from the Streptococcus (e.g., S. thermophiles), Bifidobacterium (e.g., B. longum, B. breve, B. infantis, B. breve, B. infantis), and Lactobacilbus genera (e.g., L. acidophilus, L. helveticus, L. bulgaricus, L. rhamnosus, L. plantarum, and L. paracasei). The methods may involve administering at least one probiotic bacterial strain, preferably a combination of two or more different bacterial strains, to a subject, preferably a mammal (e.g., a human). Such administration may be systemically or locally (e.g., directly to intestines) performed. A preferred administration route is oral administration. Other routes (e.g., rectal) may be also used. For administration, either the bacteria (e.g., in a wet, sonicated, ground, or dried form or formula), the bacterial culture medium containing the bacteria, or the bacterial culture medium supernatant (not containing the bacteria), may be administered.

AJ2 is a combination of eight strains of gram positive probiotic bacteria with the ability to induce synergistic production of IFN-γ when added to IL-2-treated- or IL-2+anti-CD16 monoclonal antibody-treated NK cells (anti-CD16mAb). The combination of strains is used to provide bacterial diversity in addition to synergistic induction of a balanced pro- and anti-inflammatory cytokine and growth factor release NK cells. The beneficial effects of AJ2 on immune cells are disclosed in International Patent Application WO18/112366, hereby incorporated herein by reference.

### Antibody-Dependent Cell-Cytotoxicity

Antibody-dependent cellular-cytotoxicity (ADCC), is a mechanism by which immune cells bearing the Fc receptor can kill the cells coated with the antibody upon binding of the Fc receptor to the Fc portion of the antibody. NK cells are one the subset of immune cells that can mediate ADCC through FcγRIIIA receptor also known as CD16. The mechanism by which NK cells mediate ADCC is not fully understood. When the effector cell recognizes the target by cross-linking of the Fc receptor and the antibody coating the target cell, the immunoreceptor tyrosine-based activation motifs (ITAMs) gets phosphorylated in the effector cells and leading to triggering of main downstream signaling pathways in the effector cell to kill the target cell. One of the mechanisms by which NK cells mediated ADCC can be through perforin-granzyme mediate cytotoxicity. The role of FAS ligand in ADCC is unknown but It has been shown that cross-linking of the CD16 receptor on NK cells can upregulate FAS ligand on them which may be indicative an important role of Fas/Fas-L in ADCC.

### Split Anergy

Split anergy is a maturation stage of NK cells, wherein NK cells show reduced cytotoxicity and augmented secretion of IFN-γ. Split-anergized NK cells promote differentiation of target cells via secreted and membrane-bound factors, increase tumor cell resistance to NK cell-mediated cytotoxicity, as well as inhibit inflammation due to the reduction of cytokine and chemokine production after tumor differentiation.

### Cancer Stem Cells

Cancer stem cells (CSCs) are stem cells which can create various populations of differentiated cells that define the tumor mass. CSCs are like normal stem cells, and have self-renewal capacity and also can be differentiated, but in a dysregulated manner. The existence of CSCs is described in many tumors including, but not limited to, acute myeloid leukemia, breast, prostate, melanoma, lung, colon, brain, liver, gastric and pancreatic cancer.

### Osteoclasts

Osteoclast are the bone cells responsible for the bone homeostasis by resorbing the bone. Osteoclast matures via RANKL stimulation and the process is regulated by ICAM-1. Proinflammatory signals can induce expression of ICAM-1 and RANKL on osteoclasts. These signals are mediated by subsets of immune cells. It has been shown that osteoclasts express multiple ligands for both activating and inhibitory NK cell receptors.

### MICA/MICB

Major Histocompatibility Complex Class I-Related Chains A and B (MICA/MICB) are proteins known to be induced upon stress, damage, viral infection or transformation of cells which act as a ‘kill me’ signal through the cytotoxic lymphocytes. In contrast to classical MIIC class-I molecules, this protein is not involved in antigen presenting but they are known to be a ligand for a natural killer group 2D (NKG2D) receptor, a receptor on cytotoxic cells. Engagement of NKG2D receptors triggers natural killer (NK) cell-mediated cytotoxicity and provides a costimulatory signal for CD8 T cells and γδ T cells. MICA/B were not thought to be constitutively expressed by healthy normal cells, but recently studies have shown that this protein is also expressed on surface of healthy cells such as breast, colon, liver, pancreas, stomach, bronchus, bladder and ureter in smooth muscle cells and/or myofibroblasts within stomach, small intestine, colon, bladder, cervix, fallopian tube, prostate and ureter. The differential expression of MICA/MICB based on the differentiation status of the tumor cells have not be studied. In this study, we will evaluate the expression of MICA/MICB on the undifferentiated/stem-like and differentiated oral and pancreatic tumors.

### Kit

A “kit” is any manufacture (e.g. a package or container) comprising at least one reagent, e.g. an antibody, an antibody fragment, a probe, or a small molecule, for specifically detecting and/or affecting the copy number, expression, and/or amount of a marker of the present invention. The kit may also comprise a biological reagent, such as cells (e.g., osteoclasts or cancer cells). The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

### II. Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human.

In certain embodiments, the subject is an animal model of cancer. For example, the animal model can be an orthotopic xenograft animal model of a human-derived cancer.

In various embodiments of the methods of the present invention, the subject has not undergone treatment, such as chemotherapy, radiation therapy, targeted therapy, and/or immunotherapies. In other embodiments, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

### III. Anti-Cancer Therapies

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with a therapy as disclosed herein. As described below, agents can be administered in combination therapy with, e.g., chemotherapeutic agents, hormones, antiangiogens, radiolabelled compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In other embodiments, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

### IV. Treatment Methods

Agents that upregulate immune responses can be in the form of enhancing an existing immune response or eliciting an initial immune response. Thus, enhancing an immune response using the subject compositions and methods is useful for treating cancer, but can also be useful for treating an infectious disease (e.g., bacteria, viruses, or parasites), a parasitic infection, and an immunosuppressive disease.

Immune responses can also be enhanced in an infected patient through an ex vivo approach, for instance, by removing immune cells from the patient, contacting immune cells in vitro with an agent described herein and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response. Such additional agents and therapies are described further below.

Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

Additionally, upregulation or enhancement of an immune response function, as described herein, is useful in the induction of tumor immunity.

Furthermore, the immune response can be stimulated by the methods described herein. For example, immune responses against antigens to which a subject cannot mount a significant immune response, e.g., to an autologous antigen, such as a tumor specific antigens can be induced by administering appropriate agents described herein, NK cells, CD8+ T cells, that upregulate the immune response. Similarly, an autologous antigen, such as a tumor-specific antigen, can be coadministered. In addition, the subject compositions can be used as adjuvants to boost responses to foreign antigens in the process of active immunization.

In certain embodiments, immune cells are obtained from a subject and cultured ex vivo in the presence of an agent as described herein, to expand the population of immune cells and/or to enhance immune cell activation. The immune cells may then be administered to a subject. Immune cells can be stimulated in vitro by, for example, providing to the immune cells a primary activation signal and a costimulatory signal, as is known in the art. Various agents can also be used to costimulate proliferation of immune cells. Immune cells may be cultured ex vivo according to the method described in PCT Application No. WO 94/29436. The costimulatory polypeptide can be soluble, attached to a cell membrane, or attached to a solid surface, such as a bead.

### V. Samples

In some embodiments, the methods of present invention may be performed using primary NK cells from a subject. In other embodiments, the NK cells have been transiently or stably transformed. In some embodiments, the NK cells are a representative sample of NK cells. In some embodiments, the NK cells are from a single subject. In other embodiments, the NK cells are a pool of NK cells from at least two subjects. In some embodiments, the NK cells are from a diseased subject, e.g., a subject that has cancer. In some embodiments, the NK cells are purified. In other embodiments, the assays may be performed with a bodily sample (e.g., a bodily fluid, such as blood) comprising NK cells. In some embodiments, the method of the present invention further comprises obtaining the sample (e.g., NK cells) from the subject prior to detecting or determining the presence or level of at least one marker or activity/function in the sample. In other embodiments, the method of the present invention further comprises obtaining additional samples from the subject after having tested the sample in the assays, e.g., if the subject's NK cells are determined to demonstrate sufficient activity.

In some embodiments, the cytokine (e.g., IFN-γ) or marker (e.g., CD44, CD54, MIHC class I, PD-L1, MICA, MICB, or CD8+) amount and/or activity measurement(s) in a sample from a subject is compared to a predetermined control (standard) sample. The sample may be from a healthy subject or a diseased subject. The control sample can be from the same subject or from a different subject. The control sample is typically a normal, non-diseased sample. However, in some embodiments, the control sample can be from a diseased tissue. The control sample can be a combination of samples from several different subjects. In some embodiments, the marker amount and/or activity measurement(s) from a subject is compared to a pre-determined level. This pre-determined level is typically obtained from normal samples. As described herein, a “pre-determined” marker amount and/or activity measurement(s) may be a marker amount and/or activity measurement(s) used to, by way of example only, evaluate a subject that may be selected for treatment. A pre-determined marker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In some embodiments, the amounts determined and/or compared in a method described herein are based on relative measurements, such as ratios (e.g., CD8+ T cells vs. CD4 T cells before and after expansion, CD8+ T cells vs. CD4 T cells within PBMC, measurement of differentiated cells in response to IFN-g produced by NK cells vs. measurement of differentiated cells in response to purified IFN-g (often commercially available), measurement of IFN-g vs. purified IFN-g).

The pre-determined marker amount and/or activity measurement(s) can be any suitable standard. For example, the pre-determined marker amount and/or activity measurement(s) can be obtained from the same or a different human for whom a patient selection is being assessed. In some embodiments, the pre-determined marker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of marker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a subject including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid). In some embodiments, the subject and/or control sample is selected from the group consisting of cells, cell lines, histological slides, paraffin embedded tissues, biopsies, whole blood, serum, plasma, buccal scrape, saliva, cerebrospinal fluid, and bone marrow. In some embodiments, the sample is PBMC.

The samples can be collected from individuals repeatedly over a longitudinal period of time (e.g., once or more on the order of days, weeks, months, annually, biannually, etc.).

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids. In some embodiments, certain cell types are purified based on at least one marker present on the cell surface. In some embodiments, such purification is be preceded by centrifugation to concentrate and/or separate out other types of undesired cells or proteins. In some embodiments, the markers present on the cell surface are determined by flow cytometry. In some embodiments, one marker is determined. In preferred embodiments, at least two, three, four, five, six, or seven markers are determined.

### VI. Gene Delivery

The instant inventions use gene delivery methods to introduce nucleic acid into cells (e.g., an exogenous nucleic acid molecule encoding CD16 is introduced to induce expression of CD16 in monocytes, which can then be used to activate NK cells). Any means for the introduction of a polynucleotide into mammals, human or non-human, or cells thereof may be adapted to the practice of this invention for the delivery of the various constructs of the present invention into the intended recipient. In one embodiment of the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well known and any can be selected for a particular application. In one embodiment of the present invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

### EXAMPLES

### Example 1: Materials and Methods for Examples 2-8

### Cell Lines, Reagents, and Antibodies

RPMI 1640 supplemented with 10% fetal bovine serum (FBS) (Gemini Bio-Products, CA, USA) was used for the cultures of human NK cells and monocytes. OSCCs and stem-like OSCSCs were isolated from oral cancer patient tongue tumors at UCLA, and cultured in RPMI 1640 supplemented with 10% FBS (Gemini Bio-Products, CA, USA), 1.4% antibiotic antimycotic, 1% sodium pyruvate, 1.4% non-essential amino acids, 1% L-glutamine, 0.2% gentamicin (Gemini Bio-Products, CA, USA), and 0.15% sodium bicarbonate (Fisher Scientific, PA, USA). Mia-Paca-2 (MP2) were cultured in DMEM with 10% FBS and 1% penicillin and streptomycin (Gemini Bio-Products, CA, USA). Recombinant IL-2 was obtained from NIH-BRB. Recombinant TNF-α and IFN-γ were obtained from BioLegend (San Diego, Calif., USA). Anti-MHC class I was prepared and 1:100 dilution was found to be the optimal concentration to use. PE conjugated anti-CD54, anti-CD44, anti-B7H1, anti-MICA/MICB antibody were obtained from BioLegend (San Diego, Calif., USA). Antibody against MICA/MICB was a generous gift from Dr. Jennifer Wu from Feinberg school of medicine. The human NK and monocyte purification kits were obtained from Stem Cell Technologies (Vancouver, BC, Canada).

RPMI 1640 supplemented with 10% Fetal Bovine Serum (FBS) (Gemini Bio-Products, CA) was used for the cultures of human NK cells, and oral squamous carcinoma stem-like cells (OSCSCs). RPMI 1640 supplemented with 10% Fetal Bovine Serum (FBS) (Gemini Bio-Products, CA) was used for the cultures the cells isolated from hu-BLT mice tissues. MiaPaCa-2 (MP2), PL12, BXPC3, HPAF, and Capan were cultured with DMEM supplemented with 10% FBS. DMEM supplemented with 10% FBS was used to culture pancreatic tumor cells isolated from hu-BLT mice pancreas. Recombinant IL-2 (rhIL-2) was obtained from NIH-BRB. Flow cytometry and other antibodies used in the study were obtained from Biolegend (San Diego, Calif.). Monoclonal antibodies to TNF-α and IFN-γ were prepared and 1:100 dilutions were found to be the optimal concentration to use for blocking experiments. NAC at 20 mM was prepared using sterilized distilled water at pH 7-7.2 and, was diluted using DMEM media to have final concentration of 20 nM.

Human pancreatic cancer cell lines Panc-1, MIA PaCa-2 (MP2), BXPC3, HPAF, Capan were generously provided by Dr. Guido Eibl (UCLA David Geffen School of Medicine) and PL12 was provided by Dr. Nicholas Cacalano (UCLA Jonsson Comprehensive Cancer Center). Panc-1, MP2 and BXPC3 were cultured with DMEM in supplement with 10% FBS and 2% Penicillin-Streptomycin (Gemini Bio-Products, CA). HPAF, Capan and PL12 were cultured in RMPI 1640 medium supplemented with 10% FBS and 2% Penicillin-Streptomycin. Recombinant human IL-2 was obtained from NIH-BRB. Recombinant human TNF-α rand IFN-γ were obtained from Biolegend (San Diego, Calif.). Antibodies to CD16 were purchased from Biolegend (San Diego, Calif.). Anti-MHC class I was prepared and 1:100 dilution was found to be the optimal concentration to use. Fluorochrome-conjugated human and mouse antibodies for flow cytometry were obtained from Biolegend (San Diego, Calif.). Monoclonal antibodies to TNF-α were prepared from ascites of mice injected with TN-α-hybridomas, after which the antibodies were purified and specificity determined by both ELISA and functional assays against rh TNF-α. Monoclonal IFN-γ antibodies were prepared in rabbits, purified and specificity determined with ELISA and functional assays against rIFN-γ. 1:100 dilution of anti-TNF-α and anti-IFN-γ antibodies was found to be the optimal concentration to block rhTNF-α and rhIFN-γ function. The human NK, CD3+ T cells and monocytes purification kits were obtained from Stem Cell Technologies (Vancouver, Canada). Propidium iodide and N-Acetyl Cysteine (NAC) were purchased from Sigma Aldrich (St. Louis, Mo.). Cisplatin and paclitaxel were purchased from Ronald Reagan UCLA Medical Center Pharmacy (Los Angeles, Calif.).

Purification of NK Cells and T Cells from the Human Peripheral Blood

Written informed consents, approved by UCLA Institutional Review Board (IRB), were obtained from healthy blood donors, and all procedures were approved by the UCLA-IRB. Peripheral blood was separated using Ficoll-Hypaque centrifugation, after which the white, cloudy layer, containing peripheral blood mononuclear cells (PBMC), was harvested, washed and resuspended in RPMI 1640 (Invitrogen by Life Technologies, CA) supplemented with 10% FBS and plated on plastic tissue culture dishes. After 1-2 hours of incubation, non-adherent, human peripheral blood lymphocytes (PBL) were collected. NK cells were negatively selected and isolated from PBLs using the EasySep® Human NK cell enrichment kit and T cells isolation kit, respectively purchased from Stem Cell Technologies (Vancouver, BC, Canada). Isolated NK cells were stained with anti-CD16 and anti-CD3 antibody, respectively, to measure the cell purity using flow cytometric analysis. Purified NK cells were cultured in RPMI Medium 1640 supplemented with 10% FBS (Gemini Bio-Products, CA), 1% antibiotic/antimycotic, 1% sodium pyruvate, and 1% MEM non-essential amino acids (Invitrogen, Life Technologies, CA).

### Expansion of Human NK Cells and Human T Cells

Human purified NK cells were activated with rh-IL-2 (1000 U/ml) and anti-CD16mAb (3 μg/ml) for 18-20 hours before they were co-cultured with feeder cells (osteoclasts or dendritic cells) and sAJ2 (NK:OCs or DCs:sAJ2; 2:1:4). The medium was refreshed every 3 days with RMPI containing rh-IL-2 (1500 U/ml). Human purified T cells were activated with rh-IL-2 (100 U/ml) and anti-CD3 (1 μg/ml) for 18-20 hours before they were co-cultured with/without osteoclasts and with/without sAJ2 (T:OCs:sAJ2; 2:1:4). The culture media was refreshed with rh-IL-2 (150 U/ml) every three days.

Human purified and hu-BLT enriched NK cells were activated with rh-TL-2 (1000 U/ml) and anti-CD16mAb (3 ug/ml) for 18-20 hours before they were co-cultured with feeder cells and sAJ2. The culture media was refreshed with rh-IL-2 every three days.

### NK Cells Supernatants Used for Stem Cell Differentiation

As described above, human NK cells were purified from PBMCs of healthy donors. NK cells were treated with a combination of anti-CD16mAb (3 μg/mL) and IL-2 (1,000 U/mL) for 18 hours before supernatants were removed and used for differentiation experiments. The amounts of IFN-γ produced by activated NK cells were assess with IFN-v ELISA (BioLegend, CA, USA). OSCSCs were differentiated with gradual daily addition of increasing amounts of NK cell supernatants. On average, to induce differentiation, a total of 3,500 μg. of IFN-γ containing supernatants were added for 5 days to induce differentiation and resistance of OSCSCSs to NK cell-mediated cytotoxicity and a total of 7000 μg. of IFN-γ containing supernatants were added for 7 days to induce differentiation and resistance of MP2 to NK cell-mediated cytotoxicity. Afterwards, target cells were washed with PBS, detached and used for experiments.

### Probiotic Bacteria (AJ2)

AJ2 is a combination of 8 different strains of gram-positive probiotic bacteria (Streptococcus thermophiles, Bifidobacterium longum, Bifidobacterium breve, Bifidobacterium infantis, Lactobacillus acidophilus, Lactobacillus plantarum, Lactobacillus casei, and Lactobacillus bulgaricus) are selected for their superior ability to induce optimal secretion of both pro-inflammatory and anti-inflammatory cytokines in NK cells.

### Sonicating AJ2

AJ2 was weighed and resuspended in RPMI Medium 1640 containing 10% FBS at a concentration of 10 mg/mL. The bacteria were thoroughly vortexed, then sonicated on ice for 15 seconds, at 6 to 8 amplitudes. Sonicated samples were then incubated for 30 seconds on ice. After every five pulses, a sample was taken to observe under the microscope until at least 80 percent of cell walls were lysed. It was determined that approximated 20 rounds of sonication/incubation on ice, were conducted to achieve complete sonication. Finally, the sonicated samples (sAJ2) were aliquoted and stored in a −80° C. freezer.

### Generation of Osteoclasts

Osteoclasts were generated from PBMC-purified monocytes and cultured in alpha-MEM medium, containing M-CSF (25 ng/mL) and RANK Ligand (RANKL) (25 ng/mL), for 21 days. 14 Medium was refreshed every 3 days with fresh alpha-MEM, containing M-CSF (25 ng/mL) and RANKL (25 ng/mL).

### ADCC Induction

The target cells (5×10⁵) were labeled with 50 μCi ⁵¹Cr (Perkin Elmer, Santa Clara, Calif.) and chromated for 1 hour. Following incubation, target cells were washed once to remove excess unbound ⁵¹Cr. Cells were resuspended in 1×10⁶/mL and the treated with the anti-MICA/MICB antibody or Cetaximab (3 μg/mL) and incubated for 30 minutes. Following incubation, target cells were washed again to remove excess unbound antibody and ⁵¹Cr. Labeled target cells were culture with effector cells and the cytotoxicity against target cells were assessed using ⁵¹Cr release cytotoxicity assay.

### ⁵¹Cr Release Cytotoxicity Assay

⁵¹Cr was purchased from Perkin Elmer (Santa Clara, Calif.). Standard ⁵¹Cr release cytotoxicity assays were used to determine NK cell cytotoxic function in the experimental cultures. The effector cells (1×10⁵ cells/well) were aliquoted into 96-well round-bottom micro-well plates (Fisher Scientific, Pittsburgh, Pa.) and titrated at 4 to 8 serial dilutions. Target cells (5×10⁵) were labeled with 50 μCi ⁵¹Cr (Perkin Elmer, Santa Clara, Calif.) and chromated for 1 hour. Following incubation, target cells were washed twice to remove excess unbound ⁵¹Cr. ⁵¹Cr-labeled target cells were aliquoted into the 96-well round bottom microwell plates containing effector cells at a concentration of 1×10⁴ cells/well at a top effector: target (E: T) ratio of 5:1. Plates were centrifuged and incubated for a period of 4 hours. After a 4-hour incubation period, the supernatants were harvested from each sample and counted for released radioactivity using the gamma counter. Total (containing ⁵¹Cr labeled target cells) and spontaneous (supernatants of target cells alone) release values were measured and used to calculate the percentage specific cytotoxicity. The percentage specific cytotoxicity was calculated using the following formula:

% Cytotoxicity=Experimental cpm−spontaneous cpm/Total cpm spontaneous cpm

Lytic unit (LU) 30/106 is calculated by using the inverse of the number of effector cells needed to lyse 30% of target cells×100.

### Enzyme-Linked Immunosorbent Assays (ELISAs) and Multiplex Cytokine Assay

ELISA kit for IFN-γ was purchased from BioLegend (San Diego, Calif.). ELISA was performed to detect the level of IFN-γ produced from cell cultures. The assay was conducted as described in the manufacturer's protocol. Briefly, 96-well EIA/RIA plates were coated with diluted capture antibody corresponding to target cytokine and incubated overnight at 4° C. After 16-18 hours of incubation, the plates were washed 4 times with wash 18 buffer (0.05% Tween in 1×PBS) and blocked with assay diluent (1% BSA in 1×PBS). The plates were incubated for 1 hour at room temperature, on a plate shaker at 200 rpm; plates were washed 4 times following incubation. Then, 100 μL of standards and samples collected from each culture were added to the wells and incubated for 2 hours at room temperature, on the plate shaker at 200 rpm. After incubation, plates were washed 4 times, loaded with detection antibody, and incubated for 1 hour at room temperature, on the plate shaker at 200 rpm. After 1 hour of incubation, the plates were washed 4 times; wells were loaded with Avidin-HRP solution and incubated for 30 minutes at room temperature, on the plate shaker at 200 rpm. After washing the plates 5 times with wash buffer; 100 uL of TMB substrate solution was added to the wells and plates were incubated in the dark until they developed a desired blue color (or up to 30 minutes). Then, 100 μL of stop solution (2N H₂SO₄) was added per well to stop the reaction. Finally, plates were read in a microplate reader, at 450 nm to obtain absorbance values (BioLegend, ELISA manual).

The levels of cytokines and chemokines were examined by multiplex assay, which was conducted as described in the manufacturer's protocol for each specified kit. Analysis was performed using a Luminex multiplex instrument (MAGPIX, Millipore, Billerica, Mass.) and data was analyzed using the proprietary software (xPONENT 4.2, Millipore, Billerica, Mass.).

### Surface Staining

1×10⁵ cells from each condition were stained in 100 μL of cold 1% BSA-PBS with predetermined optimal concentration of PE conjugated antibodies, as detailed in the experiments, and incubated at 4° C. for 30 minutes. Then, cells were washed and resuspended in 1% BSA-PBS. The Epics C (Coulter) flow cytometer was used for cellular surface analysis.

### Statistical Analysis

An unpaired or paired two-tailed Student's t-test were performed to compare different groups depending on the experimental design. The p-values were expressed within the figures as follows: ***p-value <0.001, **p-value: 0.001-0.01, *p-value: 0.01-0.05. The GraphPad Prism software was used to analyze the data.

The prism-7 software was also used for the statistical analysis. An unpaired or paired, two-tailed student t-test was performed for the statistical analysis. One-way ANOVA with a Bonferroni post-test was used to compare different groups. (n) denotes the number of human donors or mice. For in-vitro studies either duplicate or triplicate samples were used for assessment. The following symbols represent the levels of statistical significance within each analysis, ***(p value <0.001), **(p value 0.001-0.01), *(p value 0.01-0.05).

### Tumor Implantation in Hu-BL T Mice

Animal research was performed under the written approval of the UCLA Animal Research Committee (ARC) in accordance to all federal, state, and local guidelines. Combined immunodeficient NOD.CB17-Prkdcscid/J and NOD.Cg-Prkdcscid Il2rgtm1Wjl/SzJ (NSG lacking T, B, and natural killer cells) were purchased from Jackson Laboratory. Humanized-BLT (hu-BLT; human bone marrow/liver/thymus) mice were prepared on NSG background as described previously. To establish orthotopic tumors, mice were first anesthetized with isoflurane in combination with oxygen, and human OSCSCs tumor cells were then directly injected in the floor of mouth in suspension with 10 μl HC Matrigel (Corning, N.Y., USA) (1×106 cells). Four to five weeks after the tumor injections, mice were euthanized, and bone marrow, spleen, and peripheral blood were harvested.

Cell Isolations from Hu-BL T Mice BA, Spleen and Peripheral Blood

To obtain single-cell suspensions from BM, femurs were cut from both ends and were flushed from one end to other using RPMI 1640 media, afterward BM cells was filtered through a 40 μm cell strainer. To obtain single-cell suspensions from spleen, spleen was smashed until no big piece was left and sample was filtered through a 40 μm cell strainer and centrifuged at 1500 rpm for 5 minutes at 4° C. The pellet was re-suspended in ACK buffer to remove the red blood cells for 2-5 mins followed re-suspension in RMPI media and centrifuged at 1500 rpm for 5 minutes at 4° C. Peripheral blood mononuclear cells (PBMCs) were isolated using ficoll-hypaque centrifugation of heparinized blood specimens. The buffy coat containing PBMCs were harvested, washed and re-suspended in RPMI 1640 medium.

### Analysis of Human Pancreatic Cancer Cells Growth in Immune-Deficient (NSG) and Humanized-BLT Mice

Animal research was performed under the written approval of the UCLA Animal Research Committee (ARC). Humanized-BLT (hu-BLT; human bone marrow/liver/thymus) mice were prepared as previously described.

In vivo growth of pancreatic tumors were done by orthotopic cell implantation into 8-10 week-old NSG mice or hu-BLT mice pancreas. To establish orthotopic tumors, mice were anesthetized using isoflurane followed by 2 cm of the incision on the lower right abdomen. Once the spleen was exposed, spleen was pulled out as pancreas in lying under the spleen. Spleen was holded using sterilized forceps and the pancreas was exposed (laparotomy). Tumor cells were then transferred by direct injection with 10 μl HC Matrigel (Corning, N.Y., USA) using insulin syringe with 28 G needle in the pancreas. Mice were monitored for tumor growth by palpating the abdominal site. 7 to 10 days after the surgery mice received 1.5×10⁶ super-charged NK cells via tail vein injection. Mice were fed AJ2 (5 billion/dose) orally, similar to how humans ingest probiotics. The first dose of AJ2 was given one or two weeks before tumor implantation and was continued throughout the experiment every 48 hours. Mice were euthanized when signs of morbidity were evident. Pancreas, pancreatic tumors, bone marrow, spleen, and peripheral blood were harvested from mice at the end of the experiment or when tumor size reached 2 cm diameter.

### Analysis of Human Oral Cancer Cells Growth in Immunodeficient and Humanized Mice

To establish orthotopic tumors, mice were anesthetized using isoflurane and oral tumor cells were then injected in oral floor by direct injection with 10 μl HC Matrigel (Corning, N.Y., USA). 7 to 10 days after the oral tumor injections, mice received 1.5×10⁶ super-charged NK cells via tail vein injection. Mice were fed AJ2 (5 billion/dose) orally, similar to how humans ingest probiotics. The first dose of AJ2 was given one or two weeks before tumor implantation and was continued throughout the experiment every 48 hours. Mice were euthanized when signs of morbidity were evident. Peripheral blood was harvested from mice at the end of the experiment or when tumor size reached 2 cm diameter.

Cell Dissociation and Cell Culture of Tissues from Hu-BL T and NSG Mice

The pancreas and/or pancreatic tumor harvested from NSG and hu-BLT mice were immediately cut into 1 mm³ pieces and placed into a digestion buffer containing 1 mg/ml collagenase IV, 10 U/ml DNAse I, and 1% bovine serum albumin (BSA) in DMEM media, and incubated for 20 minutes at 37° C. oven on a 150 rpm shaker. After digestion, the sample was filtered through a 40 mm cell strainer and centrifuged at 1500 rpm for 10 minutes at 4° C. The pellet was re-suspended in DMEM media and cells were counted. To obtain single-cell suspensions from BM, femurs were cut from both ends and were flushed from one end to other using RPMI media, BM cells was filtered through a 40 mm cell. To obtain single-cell suspensions from spleen, spleen was smashed until no big piece was left and sample was filtered through a 40 mm cell and centrifuged at 1500 rpm for 5 minutes at 4° C. The pellet was re-suspended in ACK buffer to remove the red blood cells for 2-5 mins followed re-suspension in RMPI media and centrifuged at 1500 rpm for 5 minutes at 4° C. Peripheral blood mononuclear cells (PBMCs) were isolated using ficoll-hypaque centrifugation of heparinized blood specimens. The buffy coat containing PBMCs were harvested, washed and re-suspended in RPMI 1640 medium.

Purification of NK Cells, CD3+ T Cells, and Monocytes from Hu-BLT Mice

NK cells from hu-BLT mice splenocytes were isolated using the human CD56+ selection kit (Stem Cells Technologies, Canada). Monocytes from hu-BLT mice BM cells were positively selected from BM using human CD 14 isolation kit (eBioscience, San Diego, Calif.). Isolated NK cells and monocytes were stained with anti-CD16 and anti-CD14 antibody, respectively, to measure the cell purity using flow cytometric analysis.

### Generation of Osteoclasts and Expansion of Human and Hu-BLT NK Cells

Purified monocytes both form human peripheral blood and hu-BLT mice BM cells were cultured in alpha-MEM medium containing M-CSF (25 ng/mL) and RANKL (25 ng/mL) for 21 days, or otherwise specified. The medium was refreshed every 3 days with fresh alpha-MEM containing M-CSF and RANKL. Human purified and hu-BLT NK cells were activated with rh-IL-2 (1000 U/ml) and anti-CD16mAb (3 ug/ml) for 18-20 hours before they were co-cultured with osteoclasts and sonicated AJ2 for NK cells expansion. The medium was refreshed every 3 days with RMPI containing rh-IL-2 (1500 U/ml).

### In-Vitro MP2 Cancer Stem Cell Differentiation

Differentiation of MP2 tumors was conducted as described previously. NK cells were treated with a combination of anti-CD16mAb (3 μg/mL) and IL-2 (1,000 U/mL) for 18 hours before supernatants were removed and used for differentiation experiments. The amounts of IFN-γ produced by activated NK cells were assess with IFN-γ ELISA (Biolegend, CA, USA). MP2 cells were differentiated with gradual daily addition of increasing amounts of NK cell supernatants (of corresponding treatments). On average, to induce differentiation, a total of 3,500 μg of IFN-γ containing supernatants were added for 4 days to induce differentiation and resistance of MP2 tumor cells to NK cell-mediated cytotoxicity. Afterwards, target cells were washed with 1×PBS, detached and used for experiments.

### Human Single-Color Enzymatic ELISPOT Assay for IFN-γ

80 μl of anti-human IFN-γ capture antibody was added to each well of a 96-well high-protein-binding PVDF filter plate and incubated overnight at 4° C. The plate was washed with 150 μl of PBS once before adding samples into the plate. 50,000 cells in 200 μl of RPMI were added into each well and incubate at 37° C., 5% CO2 overnight. After incubation, the plate was washed twice with 200 μl PBS followed by 0.05% 200 μl Tween-PBS twice. 80 μl of anti-human IFN-γ detection antibody was added into each well and incubated at room temperature for 2 hours and the plate was washed three times with 200 μl/well of 0.05% Tween-PBS. 80 μl/well of tertiary solution which was made from 1:1000 diluted Strep-AP was added in the plate and incubated for 30 minutes. The plate was washed twice with 200 μl/well of 0.05% Tween-PBS followed by 200 μl/well distilled water twice. Then, 80 μl/well of blue development solution was added, and the plate was incubated at room temperature for 15 minutes. The reaction was stopped by gently rinsing membrane with tap water for 3 times. Air-dried the plate for 2 hours and was scanned to count IFN-γ release using CTL machine with immunoSpot® Sofeware. (Cellular Technology Limited, OH, USA).

Purification of Human Monoytes and, Generation of Osteoclasts and Dendritic Cells

Monocytes were negatively selected and isolated from PBMCs using the EasySep® Human monocyte isolation kit purchased from Stem Cell Technologies (Vancouver, BC, Canada). Isolated monocytes were stained with anti-CD14 antibody to measure the cell purity using flow cytometric analysis, greater than 95% purity was achieved. Monocytes were differentiated to osteoclasts by treating with M-CSF (25 ng/mL) and RANKL (25 ng/mL) for 21 days. To generate dendritic cells (DCs), monocytes were treated with GM-CSF (150 ng/mL) and IL-4 (50 ng/mL) for 7 days.

### Target Cell Visualization Assay (TVA)

Target cells were incubated with TVATM dye at 370 C for 15 mins, afterwards effector cells were cultured with target cells for 4 hours. After a 4-hour incubation period the target cells were counted with immunospot at 525 nm emission wavelengths. The percentage specific cytotoxicity was calculated as follows:

% Cytotoxicity=Experimental cpm−spontaneous cpm/Total cpm−spontaneous cpm

### Example 2: [1] the Cytotoxic Function of NK Cells Will be Measured Against OSCSCs and MP2 Tumors which are Very Sensitive and Specific Targets of NK Cells Unlike Gold Standard K562 Tumors Used Currently

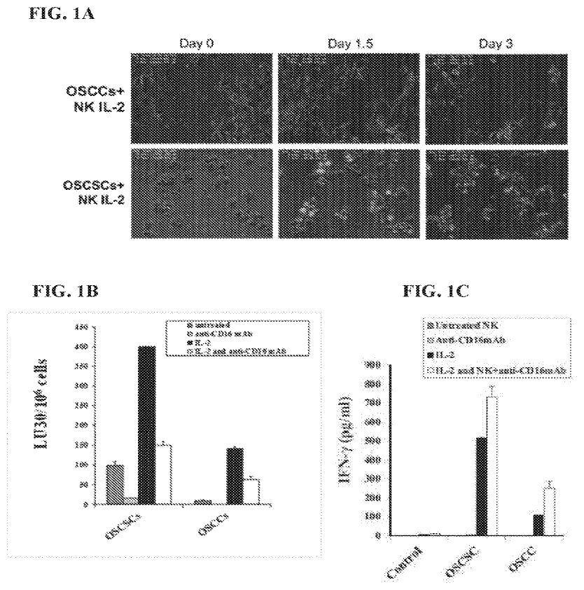

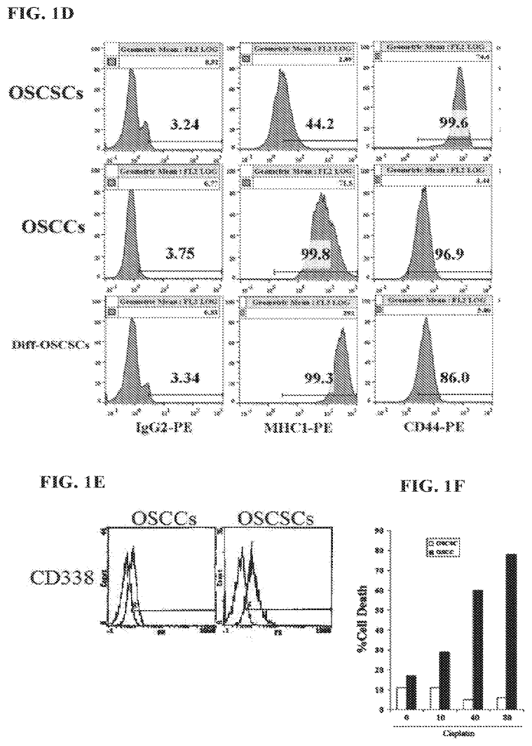

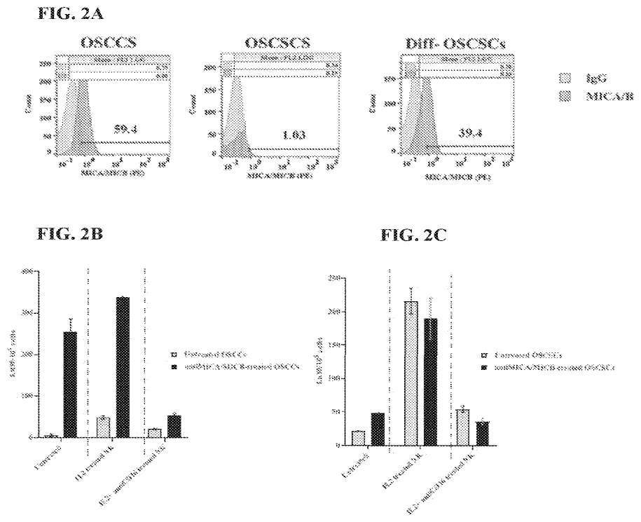

The uniqueness of this test is because of the use of NK specific tumor cells. The existing methodologies are not specific to NK cell mediated killing. K562 cells are the gold standard cells used to assess NK cytotoxicity, but these cells sometimes are killed by the T cells and they are not very specific to the function of NK cells. We identified a number of cancer stem cells/poorly differentiated tumors which are highly susceptible to direct killing by the NK cells and their respective differentiated counterparts, which are not or are less susceptible to NK cell mediated direct cytotoxicity (FIGS. 1 and 2). However, differentiated tumors become susceptible to NK cell mediated ADCC even though they are not killed directly by the NK cells since often they upregulate expression of receptors that antibodies target, therefore, NK cells can kill both undifferentiated and differentiated tumors using different mechanisms of killing (please see below for further description). The killing ability of NK cells against cancer stem cells is specific since other subsets of immune cells such as CD8+ T cells, γδ T cells and CD4+ T cells are not capable of killing these targets (see below).

### OSCSCs are Sensitive and Specific Targets of NK Cells.

Increased lysis of stem-like OSCSCs but not differentiated OSCCs by untreated, IL-2-treated, and IL-2+ anti-CD16-treated NK cells (FIGS. 1A and 1B). In addition, OSCSCs trigger IFN-g secretion from the NK cells whereas their differentiated counterparts OSCCs trigger much less (FIG. 1C). OSCSCs express higher CD44 and lower MHC class I surface receptors and are not susceptible to CDDP induced cell death unlike differentiated OSCCs potentially due to increased levels of CD338 (multidrug resistant gene) (FIG. 1D-1F).

### Differential NK Cell Mediated Lysis Through Direct Killing or ADCC Depending on the Stage of Differentiation of the Oral Tumors

OSCCs express higher levels of surface MIC A/B as compared to OSCSCs (FIG. 2A). We observed lower NK cell mediated direct killing but increased ADCC against differentiated OSCCs (FIG. 2B) whereas increased NK cell mediated direct killing were observed in the absence of ADCC against OSCSCs (FIG. 2C) in the presence of anti-MIC A/B antibody. Differentiation of OSCSCs by the NK cells increases MIC A/B expression (FIG. 2A), and increased ADCC by the NK cells (data not shown). Treatment of NK cells with IL-2 and anti-CD16mAb completely blocked NK cell mediated ADCC against OSCCs (FIG. 2B)

MP2 Stem-Like/Poorly Differentiated Pancreatic Tumors are Highly Susceptible to NK Cell Mediated Cytotoxicity Whereas their Well Differentiated Counterparts are Resistant to NK Cell Mediated Cytotoxicity

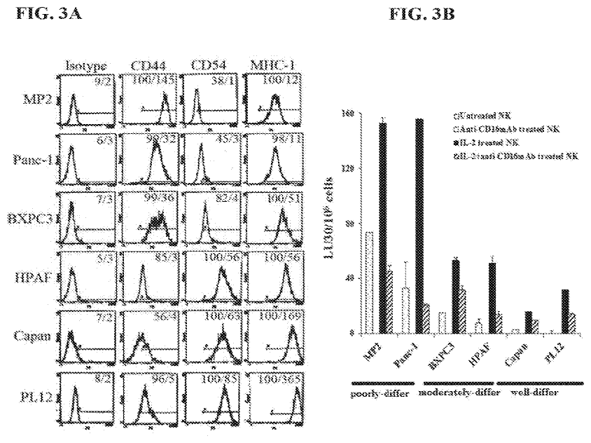

Six pancreatic tumor cells were used to determine surface expression and susceptibility to NK cell mediated cytotoxicity when cultured with NK cells. Poorly differentiated MP-2 and Panc-1 expressed higher amounts of CD44 and moderate or low levels of MHC class I and CD54. Moderately differentiated BXPC3 and HPAF expressed moderate to high levels of CD44 and CD54 and higher levels of MHC-class I when compared to MP2 and Panc-1. Well differentiated Capan and PL12 had much lower levels of CD44 and much higher levels of CD54 and MHC class I (FIG. 3A). A direct correlation between the stage of differentiation and susceptibility to NK cell mediated cytotoxicity was observed in pancreatic tumor cells. Undifferentiated MP2 and Panc-1 exhibited the highest whereas PL-12 and Capan well differentiated tumors demonstrated the lowest sensitivity to NK mediated lysis (FIG. 3B). Moderately differentiated BXPC3 and HPAF demonstrated intermediate sensitivity to NK cell lysis (FIG. 3B). Thus, there was a direct correlation between increased susceptibility to NK-mediated cytotoxicity and poor differentiation of pancreatic tumors.

### Paclitaxel Induced Significant Cell Death in Patient-Derived Differentiated PL12 and Capan Pancreatic Tumors but not in MP2 Poorly Differentiated Tumors

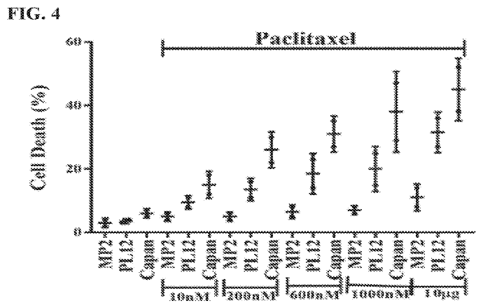

Unlike MP2 tumors, treatment of well-differentiated PL12 and Capan tumors with paclitaxel (FIG. 4) demonstrated much higher levels of cell death

### Differential NK Cell Mediated Lysis Through Direct Killing or ADCC Depending on the Stage of Differentiation of the Pancreatic Tumors

Differentiated PL-12 tumors express higher levels of surface MIC A/B as compared to MP2s (FIG. 5A). Differentiation of V1P2s by the NK cells increases MIC A/B expression (FIG. 5A). Lower NK cell mediated direct killing but increased ADCC against differentiated PL12 (FIG. 5B) whereas increased NK cell mediated direct killing in the absence of ADCC against MP2 tumors (FIG. 5C) in the presence of anti-MIC A/B antibody. Treatment of NK cells with IL-2 and anti-CD 16mAb completely blocked NK cell mediated ADCC against OSCCs (FIG. 2B). Unlike primary NK cells, NK92 cells or CD16 transfected NK92 cells (NK92V) were unable to mediate either direct killing or ADCC (FIGS. 5D and 5E).

### Only Primary Peripheral Blood Derived NK Cells Mediate Cytotoxicity Against OSCSCs

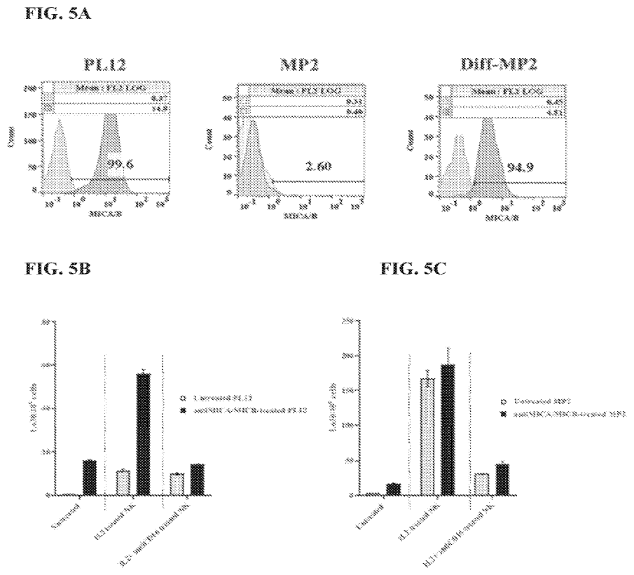

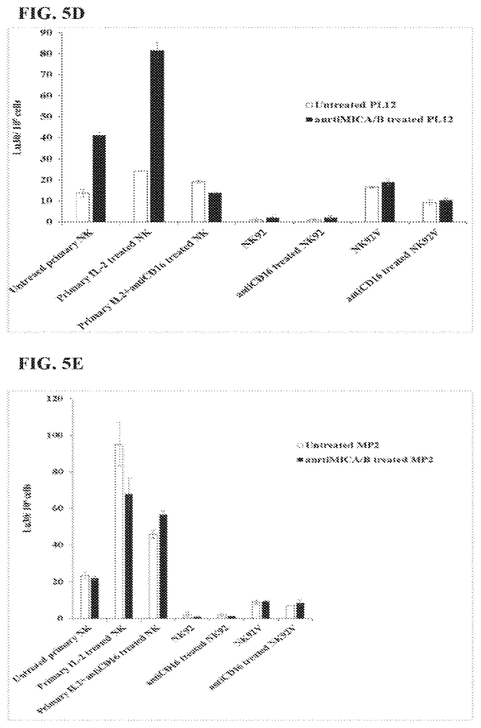

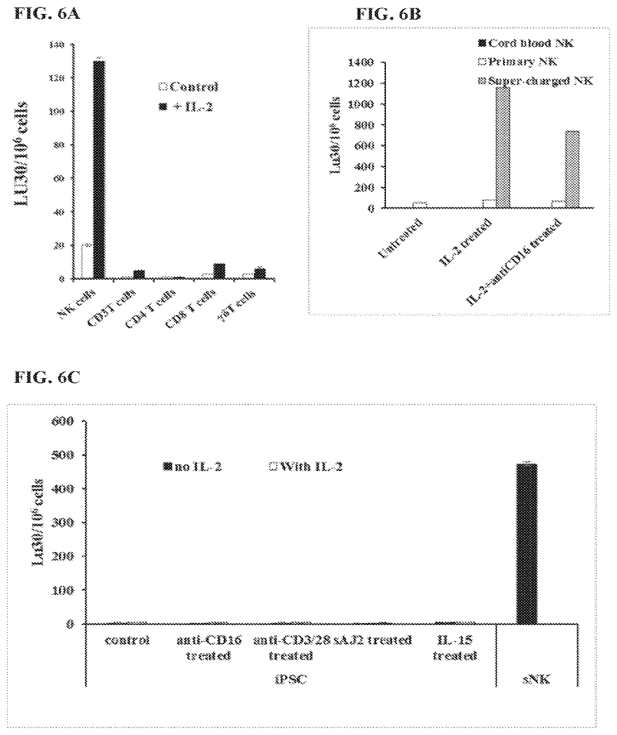

NK cells, CD3+ T cells, CD4+ T cells, CD8+ T cells and gdT cells were all sorted from the peripheral blood and activated with IL-2 before they were added to 51 Cr labeled OSCSCs Only NK cells were able to kill the OSCSCs (FIG. 6A) Primary NK cells were sorted from the peripheral blood and super-charged before both the primary NK cells and super-charged NK cells (please see below for the description of super-charged NK cells) were used in 51Cr release assay (FIG. 6B). The effect were compared to cord blood derived NK cells (FIG. 6B). Cord blood derived NK cells were unable to kill OSCSCs (FIG. 6B). Super-charged NK cells killed OSCSCs at very high levels (FIG. 63). iPSC derived NK cells were also devoid of killing OSCSCs whereas very high levels of NK cell mediated killing of OSCSCs were observed in the presence of super-charged NK cells (FIG. 6C). These experiments demonstrate the fine specificity of peripheral blood NK cells in lysis of specific NK cell targets unlike K562 tumors which have been used in the literature to demonstrate cytotoxicity by cord blood derived NK cells and iPSC derived NK cells. Tberefore, our protocol provides a very specific system to determine the NK cell cytotoxicity.

Functional Loss of NK Cells Obtained from Peripheral Blood of Cancer Patients

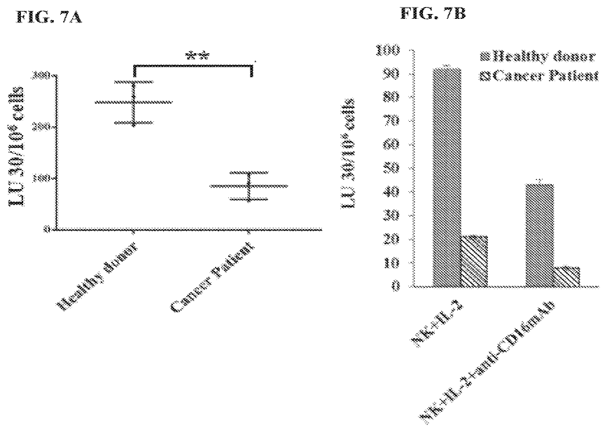

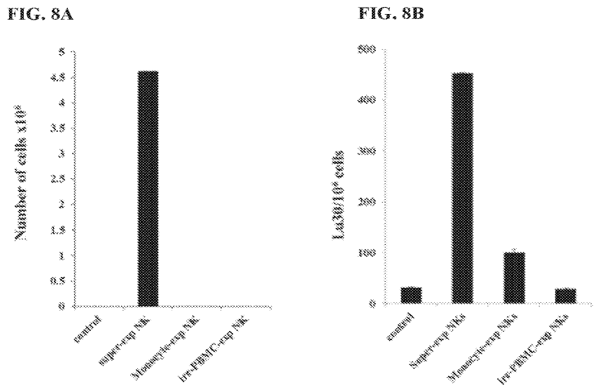

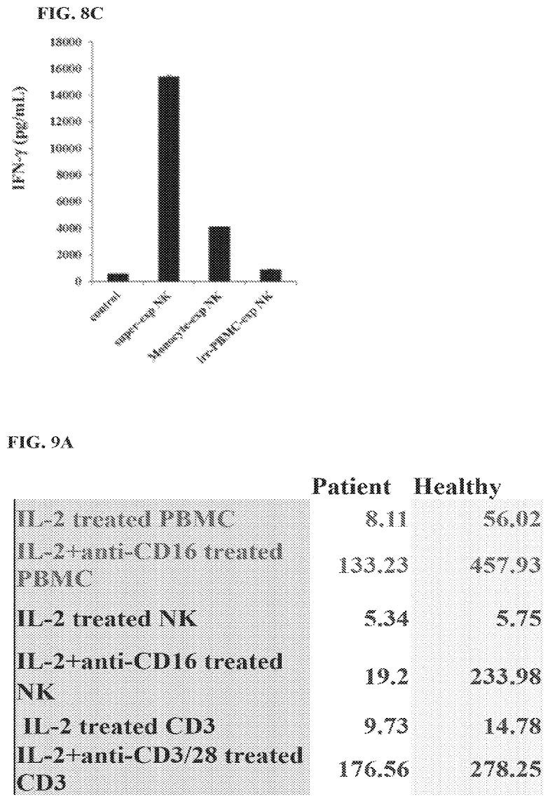

Purified primary NK cells from peripheral blood of cancer patients have significantly less capability to kill OSCSCs when compared those isolated from healthy individuals' peripheral blood (FIGS. 7A and B)

Osteoclast Expanded Super-Charged NK Cells have Superior Levels of Cytotoxicity and Secretion of IFN-g

Super charge NK cells have superior expansion capability as well as increased cytotoxicity and secretion of IFN-g when compared to monocyte expanded NK cells or irradiated PBMC expanded NK cells.

### Example 3: [2] Both ELISA and Elispot Will be Used to Assess the Levels of IFN-2 Induced in NK Cells Obtained from Healthy and Cancer Patients

Assessment with both methodologies are more precise and determines overall as well as per cell basis of NK cells

### Patients' PBMCs and NK Cells Produce Lower Levels of IFN-g Under Different Treatment Conditions

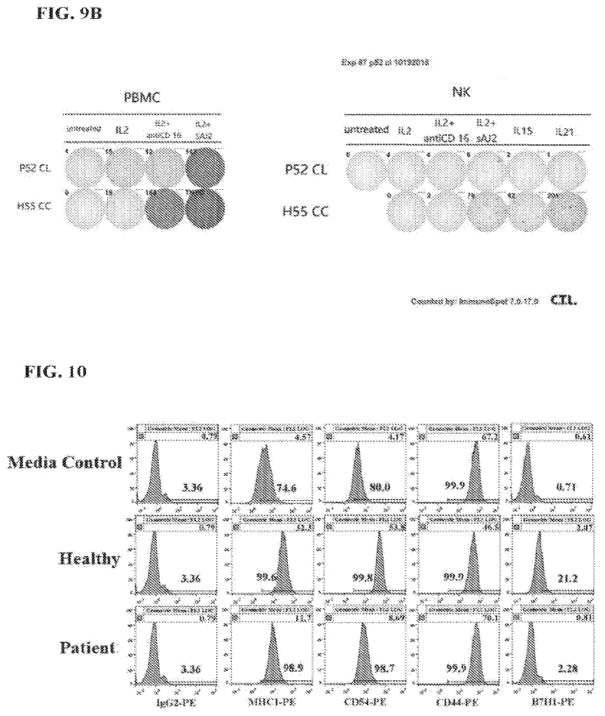

Patient PBMCs and purified NK cells produce much lower levels of IFN-g when compared to those obtained from healthy individuals as assessed with both Elisa (FIG. 9A) and Elispot (FIG. 9B)

### Example 4: [3] the Ability of Secreted IFN-2 to Differentiate Tumors Will be Assessed

We determined that IFN-g and TNF-α together can synergize to increase differentiation of tumor cells to limit tumor growth and increase surface markers of CD54, MHC class I and PDL-1. Since the same amount of IFN-g secreted from healthy individuals and those of cancer patients mediate differential levels of differentiation as assessed by upregulation of differentiation antigens listed above, being much less in NK cells from cancer patients, this will determine the functional ability of IFN-g secreted from the NK cells in differentiation of tumors in cancer patients. As can be seen in FIG. 10 IFN-g secreted from patient's NK cells had much lower capacity to increase differentiation antigens of MHC class I, CD54 and B7H1 when compared to those secreted from the healthy individuals NK cells

### Example 5: [4] the Ability of Patient NK Cells to be Expanded by Autologous and Allogeneic Osteoclasts Will be Assessed and if Low then Allogeneic NK Cells Will be Used, Both Cytotoxicity and IFN-2 Secretion and Ability to Differentiate Tumors Will be Tested

This test will determine whether cancer patients NK cells can be expanded, and whether expanded NK cells will be functional in terms of cytotoxicity (both direct killing and ADCC) and that they will produce functional IFN-g. If it was found that they do not expand to the therapeutic levels then allogeneic NK cells will be used for immunotherapy.

Suppression of Primary NK Cell-Mediated Cytotoxicity and/or Secretion of Cytokines in Cancer Patients

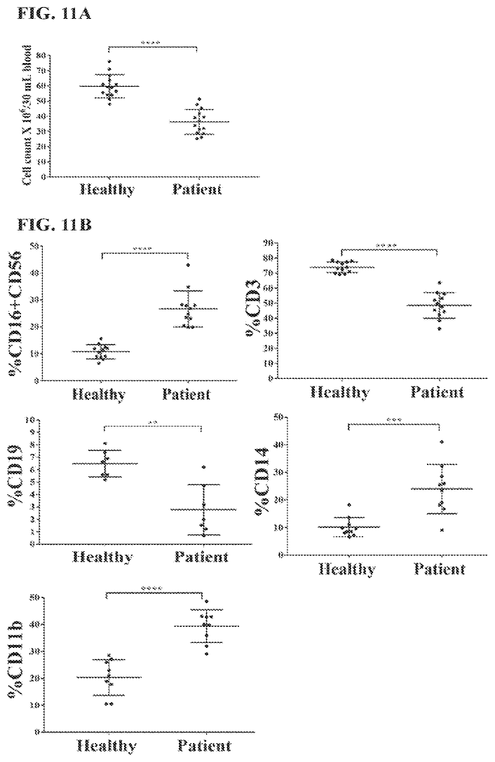

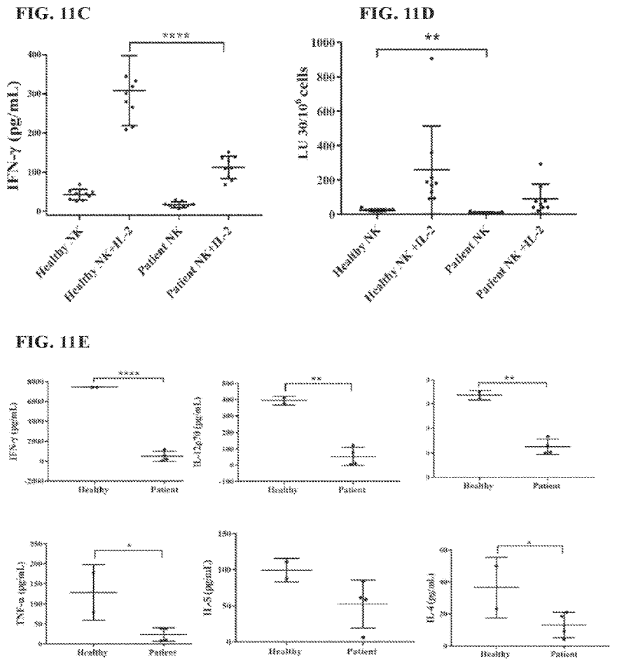

Lower numbers of PBMCs were recovered from the peripheral blood of cancer patients when compared to those isolated from healthy individuals (FIG. 11A). Higher percentages of CD16+CD56+, CD14+, CD11b+ cells, and low percentages of CD3+, and CD19+ cells were obtained within CD45+PBMCs isolated from cancer patients in comparison to healthy individuals (FIG. 111B). NK cells from cancer patients exhibited decreased IFN-γ secretion (FIGS. 11C and 11E) and, significantly lower NK cell mediated cytotoxicity when compared to NK cells from healthy individuals (FIG. 11D). In addition, similar to IFN-γ, secretion of IL-12p70, IL-6, TNF-α, IL-5, and IL-4 were also significantly lower from cancer patients' NK cells when compared to those from healthy individuals (FIG. 11E). Similar results were seen in the sera collected from peripheral blood of the cancer patients (FIG. 11F).

Super-Charged NK Cells from Cancer Patients have Much Lower Capacity to Expand, or Mediate Cytotoxicity and Secrete IFN-γ Compared to Healthy Individuals.

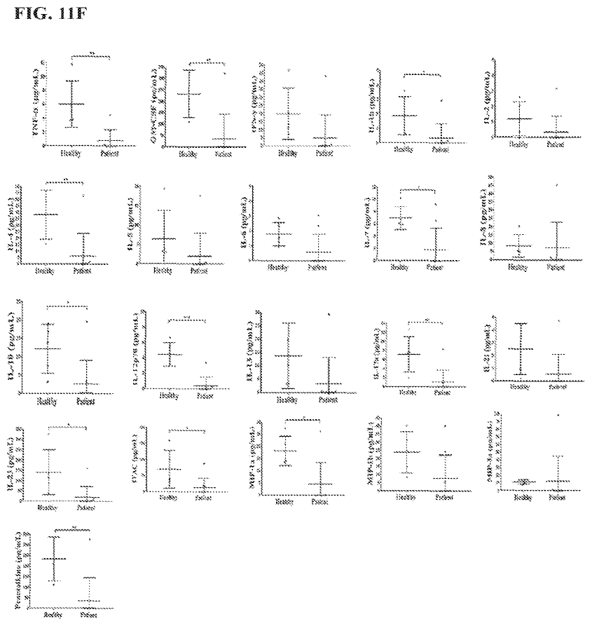

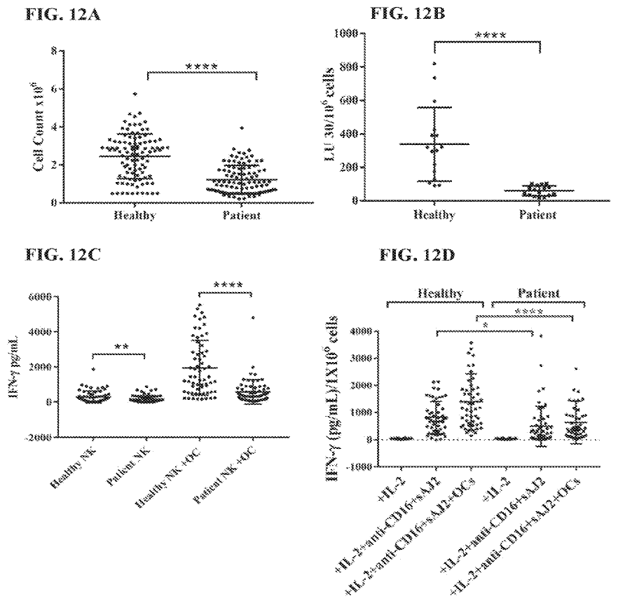

Purified NK cells from cancer patients and healthy individuals were cultured with healthy allogeneic OCs, and the levels of NK cell expansion, cytotoxicity and IFN-γ secretion were assessed. NK cells from cancer patients had significantly lower expansion (FIG. 12A), and lower NK cell-mediated cytotoxicity (FIG. 12B) compared to those from healthy individuals (FIGS. 12A-12B). Cancer patients' NK cells also mediated significantly lower levels of IFN-γ secretion both in the absence or presence of OCs (FIGS. 12C-12D). Similar to NK cells, T cells from cancer patients had defects which we can determine by using our methodologies (data can be provided if needed)

### Example 6: [5] Determining the Ability of NK Cells to Expand Functional CD8+ T Cells

Our recent findings indicated that NK cells are important in selection and expansion of CD8+ T cells by elimination of CD4+ T cells. We will perform this test to determine how well NK cells are able to expand CD8+ T cell expansion

### Super-Charged NK Cells Expanded CD8+ T Cells Significantly

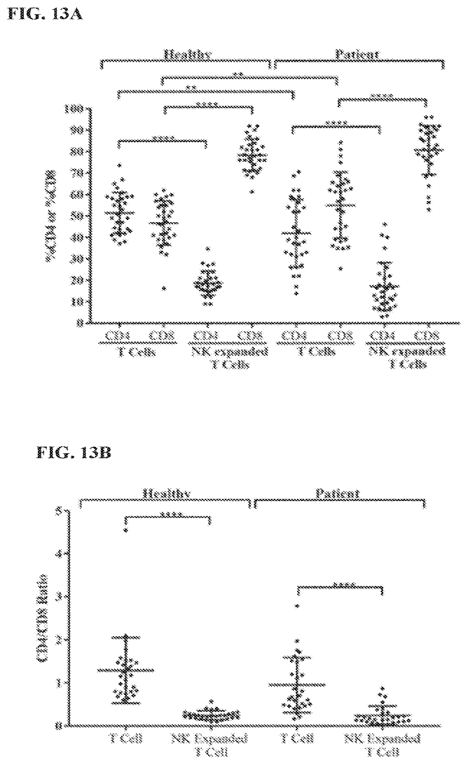

We next cultured each of purified NK cells and T cells with OCs and determined the fractions of CD4+ and CD8+ T cells within both the NK and T cell co-cultures with OCs. Purified T cells cultured with OCs increased the percentages of CD8+ T cells and the ratio of CD4/CD8 decreased from 2.4 in T cells in the absence of OCs to 1.2 in those cultured with OCs (FIG. 13A). In contrast, T cells expanded within NK cultures with OCs significantly increased the percentages of CD8+ T cells and accordingly the ratio of CD4/CD8 decreased substantially (FIG. 3E). Similar trends were also seen when T cells and NK cells from the patients were cultured with OCs, except T cells cultured with OCs from patients had lower CD4/CD8 ratios when compared to healthy controls (FIG. 13B). Thus, purified T cells isolated from healthy individuals and cancer patients in the absence of NK cells failed to expand CD8+ T cells significantly, although cancer patients T cells had higher percentages of CD8+ T cells constitutively (FIGS. 13A-13B). In addition, purified NK cells activated with OCs which contained undetectable or negligible levels of contaminating T cells at the start of the culture, expanded CD8+ T cells at the later times during the expansion period from both healthy and patient cultures, albeit patient NK cell cultures expanded CD8+ T cells faster than healthy NK cells (FIG. 13).

Immunotherapy with NK Cells Increased CD8+ T Cells and, Resulted in an Increase in IFN-γ Secretion and NK Cell-Mediated Cytotoxicity in Oral Tumor-Bearing Hu-BLT Mice

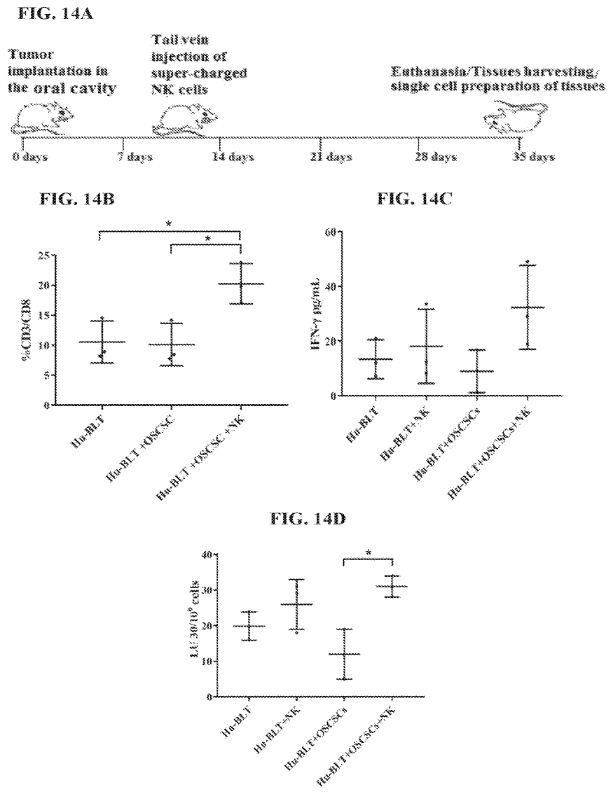

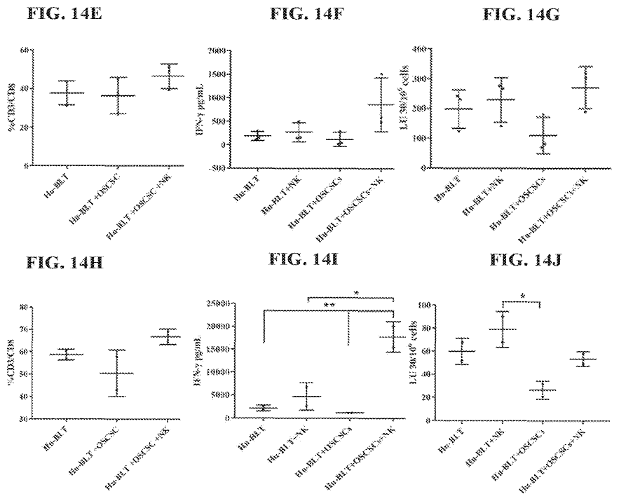

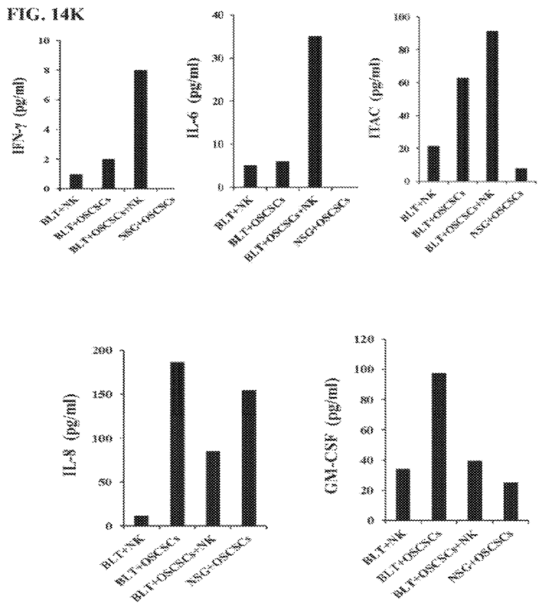

Hu-BLT mice were implanted with OSCSCs in the oral cavity and injected with super-charged NK cells with potent cytotoxic and cytokine secretion capabilities. After several weeks mice were sacrificed and tissues were removed, dissociated and the cells were analyzed (FIG. 14A). Increased proportions of CD3*CD8-T cells within BM (FIG. 14B), spleen (FIG. 14E) and peripheral blood (FIG. 14H) were seen in NK-injected tumor-bearing mice as compared to the other groups. Injection of NK cells resulted in an increased IFN-γ secretion from BM (FIG. 5C), spleen (FIG. 14F) and peripheral blood (FIG. 14I), and increased NK cell-mediated cytotoxicity in BM (FIG. 14D), spleen (FIG. 14F), and peripheral blood (FIG. 14J) in tumor-bearing mice when compared to those in the absence of NK cell injection. Interestingly, sera from peripheral blood of NK-injected tumor-bearing mice exhibited increased IFN-γ, IL-6 and ITAC, but decreased IL-8 and GM-CSF in NK-injected tumor-bearing mice when compared to tumor-bearing mice in the absence of NK cells injection (FIG. 14K).

### CD8+ T Cells Expanded by Super-Charged NK Cells Secrete Higher Levels of Cytokines

CD8+ T cells expanded by super-charged NK cells and sorted after 12 days of expansion secrete higher levels of cytokines when compared to those obtained from OC expanded CD8+ T cells. GM-CSF, sCD137, IFN-g, IL-10, sFASL and TNF-α were higher in CD8+ T cells sorted out from super-charged NK cells whereas no difference could be seen for Granzyme B and secreted Fas and lower levels of Granzyme A and perforin could be observed. These results suggest that NK cells increase cytokine release by the CD8+ T cells while decreasing the release of granules.

### Example 7: [6] Measuring the Expression and Function of CD16 Receptor

Many patients have lower expression of CD16 receptor and defective CD16 function, this will be tested for the secretion of IFN-g and ability to mediate ADCC function against differentiated tumors (please see above) since if they have dysfunctional CD16 expression and function different strategies for the immunotherapy of these patients should be employed.

Activation Through CD16 Receptor does not Trigger IFN-g Secretion from the Cancer Patients

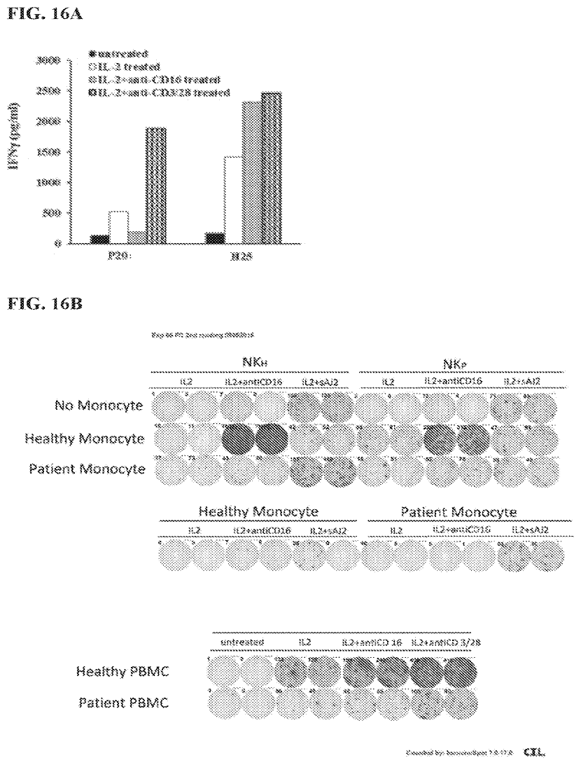

PBMCs and Purified NK cells isolated from cancer patients do not respond to CD16 mediated signaling to upregulate IFN-g secretion both in Elisa (FIG. 16A) and in Elispot (FIG. 16B). The defect is not only due to the dysfunction of NK cells but also for the lack of ability of patient monocytes to synergize with NK cells to induce IFN-g. These results indicated that patients have significant defect in CD16 mediated signaling for the increase secretion of IFN-g. More importantly patients' Monocytes do not have defect in triggering NK cell mediated IFN-g release activated by our patented probiotic bacteria sAJ2 which can serve significant treatment strategy. In addition, patient T cells are still capable of activation through CD3 and CD28. Therefore, these experiments will allow us to clearly demonstrate which cell type is defective and which receptor is defective in cancer patients and tail make the immunotheraputic strategies accordingly.

### Example 8: [7] Determining the Status of Patient Tumor Cells with Our Biomarkers of Differentiation

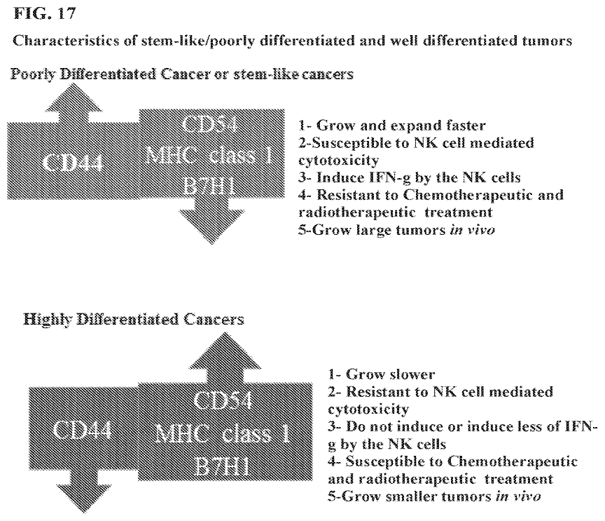

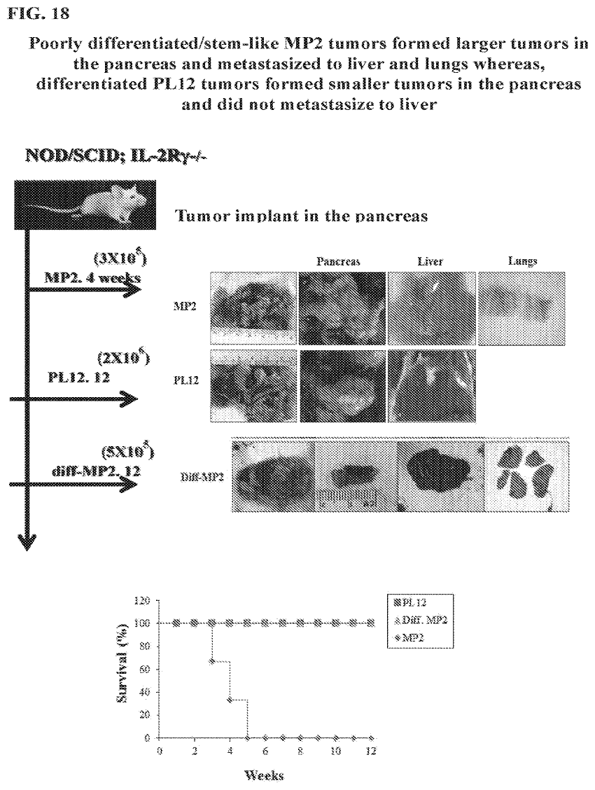

We have established biomarkers of tumor differentiation within the tumors. The biomarkers to be tested are CD44, CD54, MHC class I and PD-L1 (B7H1). Higher CD44 and lower CD54, MHC class I and PD-L I will establish the poorly differentiated nature of the tumor cells and lower CD44 and higher CD54, MHC class I and PD-L1 will establish the higher differentiation of tumor cells (Please see FIG. 1-3). We will also include MICA/B as a differentiation agent (FIG. 2). Establishing the levels of differentiation will also be important to predict the efficacy of chemotherapy treatment, as shown above. These tests will be important for the prognosis. Please see below for the characteristics of well differentiated vs. poorly differentiated tumors (FIG. 17) and the protocol for tumor implantation in NSG mice (FIG. 18). As can be seen (FIG. 18) smaller numbers of MP2 grew larger tumors with metastasis whereas higher numbers of PL12 only grew small tumors with no metastasis. Differentiation with NK cells before implantation of MP2 did not grow any tumors even when used at a higher numbers than the parental lines without NK differentiation.

### Example 9: Materials and Methods for Examples 10-19

### Cell Lines, Reagents, and Antibodies

RPMI 1640 supplemented with 10% fetal bovine serum (FBS) (Gemini Bio-Products, San Diego, Calif., USA) was used for the cultures of human NK cells. Human pancreatic cancer cell lines Panc-1, MIA PaCa-2 (MP2), BXPC3, HPAF, and Capan were generously provided by Dr. Guido Eibl (UCLA David Geffen School of Medicine) and PL12 was provided by Dr. Nicholas Cacalano (UCLA Jonsson Comprehensive Cancer Center). Panc-1, MP2, and BXPC3 were cultured with DMEM supplemented with 10% FBS and 1% Penicillin-Streptomycin (Gemini Bio-Products, West Sacramento, Calif., USA). HPAF, Capan and PL12 were cultured in RMPI 1640 medium supplemented with 10% FBS and 1% penicillin-streptomycin. Recombinant human IL-2 was obtained from NIH-BRB. Human TNF-α and IFN-γ was obtained from Biolegend (San Diego, Calif., USA). Antibody to CD16 was purchased from Biolegend (San Diego, Calif., USA). Fluorochrome-conjugated human and mouse antibodies for flow cytometry were obtained from Biolegend (San Diego, Calif., USA). Monoclonal antibodies to TNF-α and IFN-γ were prepared in our laboratory, and used at 1:100 dilutions to block rhTNF-α and rhIFN-γ functions. The human NK cell and monocyte purification kits were obtained from Stem Cell Technologies (Vancouver, BC, Canada). Propidium iodide (PI) and N-Acetyl Cysteine (NAC) were purchased from Sigma Aldrich (St. Louis, Mo., USA). Cisplatin and paclitaxel were purchased from Ronald Reagan UCLA Medical Center Pharmacy (Los Angeles, Calif., USA).

### Ethics Approval and Consent to Participate

Written informed consents approved by UCLA Institutional Review Board (IRB) were obtained and all procedures were approved by the UCLA-IRB (IRB #11-000781). Animal research was performed under the written approval of the UCLA Animal Research Committee (ARC) (protocol #2012-101-13).

### Purification of Human NK Cells and Monocytes

NK cells and monocytes were negatively selected from PBMCs using isolation kits from Stem Cell Technologies (Vancouver, BC, Canada). Greater than 96% purity was obtained both for purified NK cells and monocytes based on flow cytometric analysis.

### Analysis of Human Pancreatic Cancer Cell Growth in Immune-Deficient (NSG) and Humanized-BLT Mice

Humanized-BLT (hu-BLT; human bone marrow/liver/thymus) mice were generated as previously described. In vivo growth of pancreatic tumors was performed by orthotopic tumor implantation in the pancreas of NSG or hu-BLT mice. To establish orthotopic tumors, mice were anesthetized using isoflurane, and tumors in a mixture with Matrigel (10 μL) (Corning, N.Y., USA) were injected in the pancreas using insulin syringe. Mice received 1.5×10⁶ super-charged NK cells via tail vein injection 7 to 10 days after the tumor implantation. They were also fed AJ2 (5 billion/dose) orally. The first dose of AJ2 was given one or two weeks before tumor implantation, and feeding was continued throughout the experiment at an interval of every 48 h. Mice were euthanized when signs of morbidity were evident. Pancreas, pancreatic tumors, bone marrow, spleen, and peripheral blood were harvested and single cell suspensions were prepared from each tissue as described previously and below.

Cell Dissociation and Cell Culture of Tissues from Hu-BLT and NSG Mice

Pancreatic tumors were harvested from NSG and hu-BLT mice and cut into 1 mm³ pieces and placed into a digestion buffer containing 1 mg/mL collagenase IV, 10 U/mL DNAse I, and 1% bovine serum albumin (BSA) in DMEM media for 20 min at 37° C. The samples were then filtered through a 40 mm cell strainer and centrifuged at 1500 rpm for 10 min at 4° C. To obtain single-cell suspensions from BM, femurs were flushed using media, and filtered through a 40 μm cell strainer. Spleens were removed and single cell suspensions were prepared and filtered through a 40 μm cell strainer and centrifuged at 1500 rpm for 5 min at 4° C. The pellets were re-suspended in ACK buffer to remove the red blood cells. Peripheral blood mononuclear cells (PBMCs) were isolated using ficoll-hypaque centrifugation.

Isolations of NK Cells, T Cells and Monocytes from Hu-BLT Mice

NK cells and T cells from hu-BLT splenocytes were obtained as described previously by using the human CD56+ and CD3+ selection kits respectively (Stem Cells Technologies, Vancouver, BC, Canada). Monocytes from hu-BLT bone marrow were isolated using human CD14 isolation kit (eBioscience, San Diego, Calif., USA).

### Generation of Osteoclasts and Expansion of Human and Hu-BLT NK Cells

Monocytes were purified form human peripheral blood or hu-BLT BM and cultured using alpha-MEM medium containing M-CSF (25 ng/mL) and RANKL (25 ng/mL) for 21 days (medium was refreshed every 3 days). NK cells were activated with rh-IL-2 (1000 U/mL) and anti-CD16 mAb (3 μg/mL) for 18-20 h before they were cultured with osteoclasts and sonicated-AJ2 to generate super-charged NK cells. The medium was refreshed every 3 days with RNPI containing rh-IL-2 (1000 U/mL).

### In-Vitro MP2 and OSCSCs Cancer Stem Cell Differentiation

Differentiation of MP2 and OSCSCs (oral squamous carcinoma stem-cells) tumors was conducted as described previously. Briefly NK cells were treated with a combination of anti-CD16 mAb (3 μg/mL) and IL-2 (1000 U/mL) for 18 h before the supernatants were removed and used for differentiation of the tumors. The amounts of IFN-γ produced by activated NK cells were assessed using ELISA kits purchased from Biolegend (San Diego, Calif., USA). To induce differentiation of tumors a total of 3500 μg of IFN-γ containing supernatants were added for 4 days.

### Enzyme-Linked Immunosorbent Assays (ELISAs) and Multiplex Cytokine Assay

Human ELISA kits for IFN-γ and IL-6 were purchased from Biolegend (San Diego, Calif., USA). The assays were conducted as recommended by the manufacturer. For certain experiments multiplex arrays were used to determine the levels of secreted cytokines and chemokines. Analysis was performed using MAGPIX (Millipore, Danvers, Mass., USA) and data was analyzed using xPONENT 4.2 (Luminex, Austin, Tex., USA).

### Surface Staining and Cell Death Assays

Staining was performed by staining the cells with antibodies as described previously, briefly, antibodies were added to 1×10⁴ cells in 50 μL of cold-PBS+1% BSA and cells were incubated on ice for 30 min. Thereafter cells were washed in cold PBS+1% BSA and flow cytometric analysis was performed using Beckman Coulter Epics XL cytometer (Brea, Calif., USA) and results were analyzed in FlowJo vX software (Flowjo, Ashland, Oreg., USA).

### ⁵¹Cr Release Cytotoxicity Assay

The ⁵¹Cr release assay was performed as described previously. Patient-derived OSCSCs were used as a specific and sensitive NK targets to assess NK cell-mediated cytotoxicity. Briefly, different numbers of effector cells were incubated with ⁵¹Cr-labeled OSCSCs. After 4 h incubation the supernatants were harvested from each sample and counted on a gamma counter. The percentage specific cytotoxicity was calculated using the following formula:

$$\% Cytotoxicity = \frac{Experimental cpm - Spontaneous cpm}{Total cpm - Spontaneous cpm}$$

Lytic unit 30/10⁶ is calculated by using the inverse of the number of effector cells needed to lyse 30% of tumor target cells×100.

### Statistical Analysis

An unpaired, two-tailed Student t-test was performed for the statistical analysis. One-way ANOVA using Prism-7 software (Graphpad Prism, San Diego, Calif., USA) was used to compare different groups. (n) denotes the number of mice used for each condition in the experiment. The following symbols represent the levels of statistical significance within each analysis, *** (p-value <0.001), ** (p-value 0.001-0.01), * (p-value 0.01-0.05).

### Example 10: Correlation between NK Cell Cytotoxicity and the Stage of Differentiation in Pancreatic Tumors

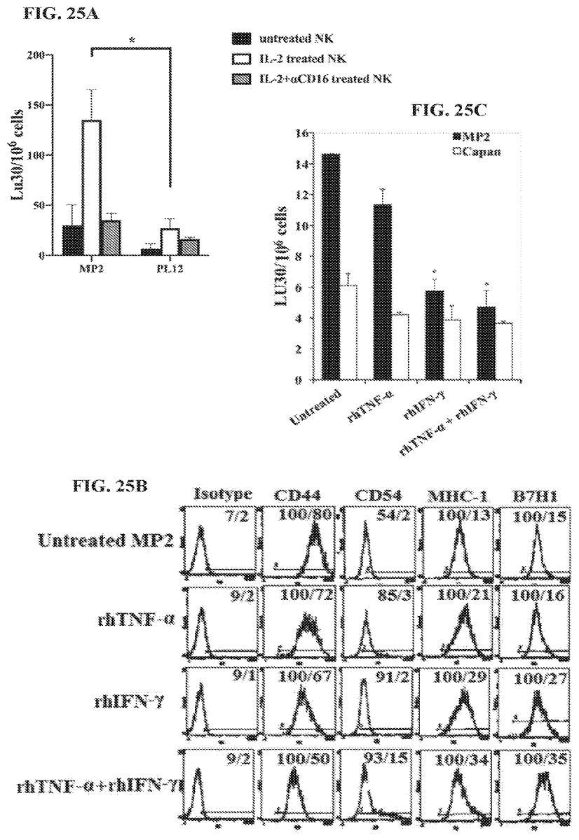

Six different pancreatic tumor cell lines each characterized at poorly, intermediate, and well differentiated stages pathologically were used to determine phenotype, susceptibility to NK cell-mediated cytotoxicity and secretion of IFN-γ directly correlating with the differentiation stages of the tumors. Poorly differentiated VIP2 and Panc-1 demonstrated moderate to low levels of MHC-class I and CD54 in the presence of higher surface expression of CD44 receptors. Moderately differentiated BXPC3 and HPAF exhibited higher levels of MHC-class I surface expression in the presence of moderate to high expression of surface CD44 and CD54 receptors, and well-differentiated Capan and PL12 expressed higher levels of surface CD54 and MHC-class I in the presence of lower CD44 surface expression (FIG. 3A). Furthermore, the stage of differentiation of the tumors was correlated with sensitivity to NK cell mediated cytotoxicity in pancreatic tumor cells. The highest susceptibility to NK cell mediated cytotoxicity was seen with undifferentiated MP2 and Panc-1 tumors; whereas the well differentiated PL12 and Capan tumors demonstrated the lowest sensitivity to NK mediated lysis (FIG. 3B and FIG. 25A). BXPC3 and HPAF, being moderately differentiated tumors, exhibited intermediate sensitivity to NK cell lysis (FIG. 3B). Therefore, a direct correlation between augmented sensitivity to NK mediated lysis and poor differentiation of pancreatic tumors was evident from these experiments. Identical results in tumor differentiation were seen in oral and glioblastoma tumors, and more recently in melanoma and lung tumors. Similar to PL12 and Capan, NK-differentiated MP2 tumors exhibited identical surface receptor phenotype, and were resistant to NK cell mediated cytotoxicity. NK cells induced differentiation of MP2 tumors through the functions of IFN-γ and TNF-α, with rhIFN-γ and/or rhTNF-α exhibiting similar results to NK-induced IFN-γ and TNF-α (see Example 17 and FIG. 25).

### Example 11: Curtailed Pancreatic Tumor Growth and Long-Term Survival of Mice after Implantation of NK-Differentiated MP2 and Patient-Derived Differentiated PL12 Tumors

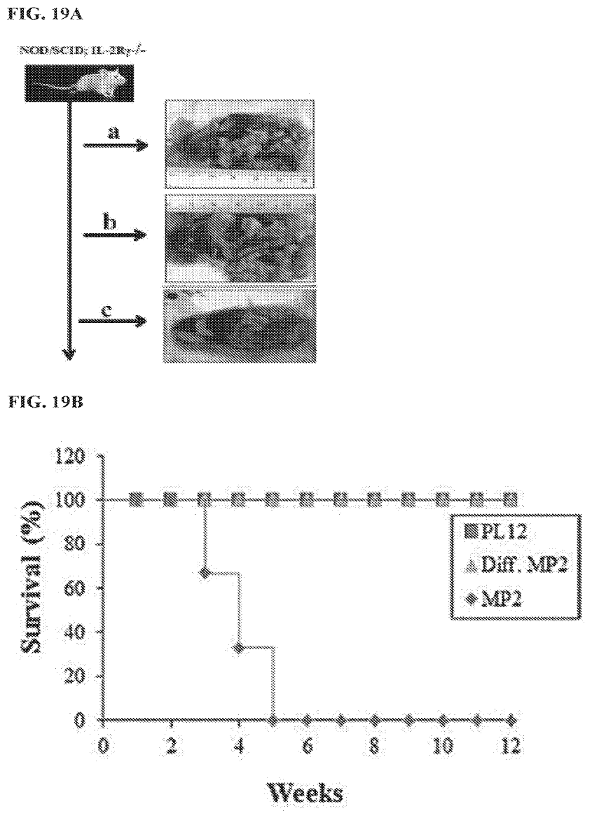

MP2 tumors (3×10⁵) implanted in the pancreas of NSG mice grew within 4 weeks and metastasized to the liver and caused significant morbidity and mortality in the mice (FIG. 18 and FIG. 19), whereas mice injected with greater numbers of PL12 tumors (2×10⁶) generated no or very small tumors within 12 weeks and the tumors did not metastasize nor caused morbidity in the mice (FIG. 18 and FIG. 19). Injection of NK-differentiated MP2 tumors (5×10⁵) to pancreas of NSG mice neither exhibited visible tumor growth nor tumors metastasized to the liver, and all mice survived at 12 weeks when the experiments were terminated (FIG. 18 and FIG. 19).

### Example 12: NK-Differentiated MP2 Tumors Did not Grow Visible Tumors in the Pancreas of Hu-BLT Mice

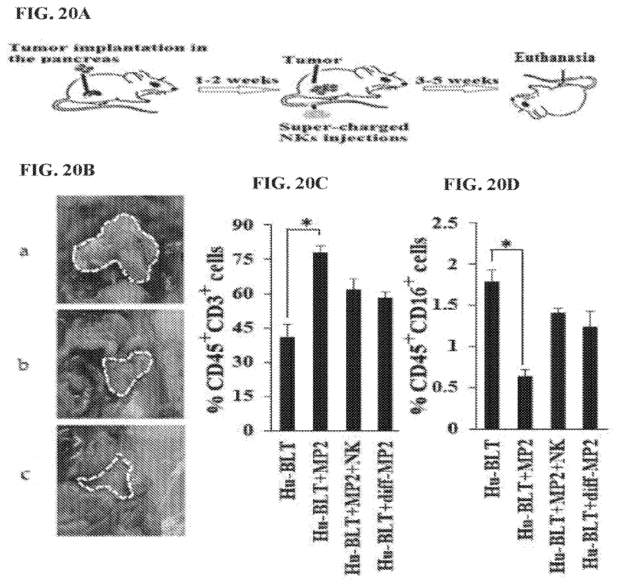

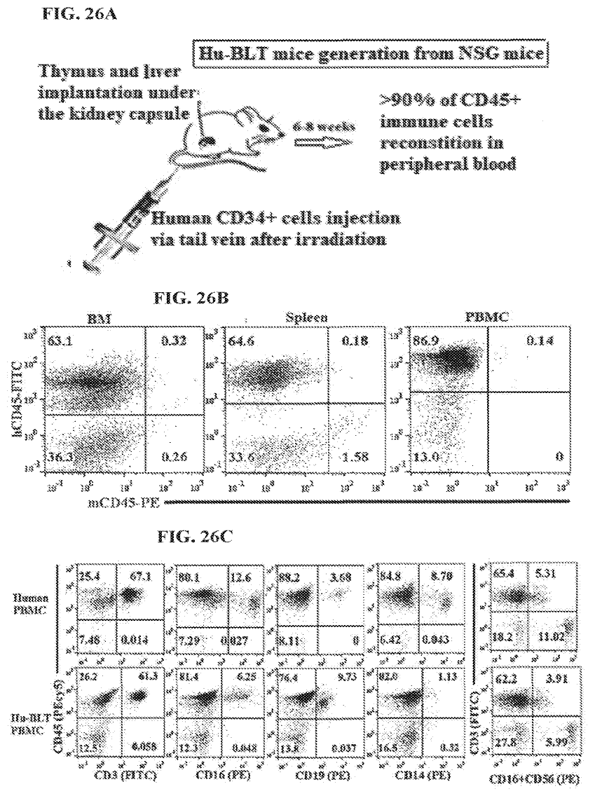

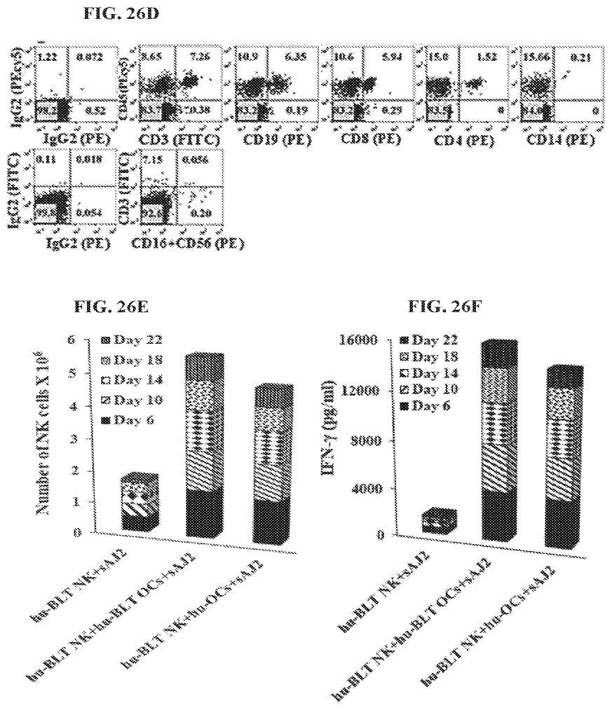

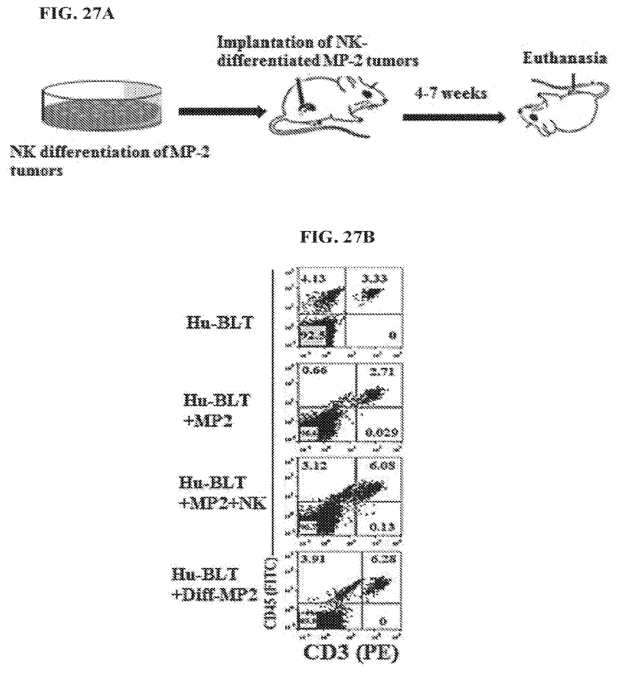

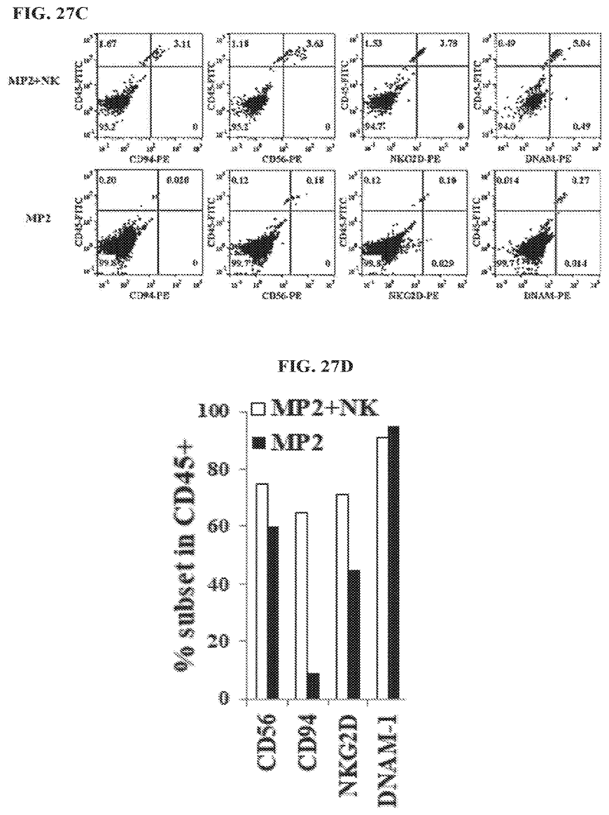

Hu-BLT mice were generated (FIG. 26B), and the successful reconstitution of human immune cells in spleen, bone marrow, and peripheral blood (FIG. 26C) were verified, and the levels of different immune subsets in peripheral blood (FIG. 26D) and pancreas (FIG. 26E) were determined, and the results were compared to peripheral blood from human donors (FIG. 26D). Hu-BLT NK cells purified from the spleen of mice responded to the activation signals provided by the IL-2 and anti-CD16 mAb treatment and expanded greatly, and demonstrated increased secretion of IFN-γ when cultured with both autologous and allogeneic osteoclasts in the presence of sAJ2 treatment (FIG. 26F and FIG. 26G), indicating close similarity between hu-BLT and human donor derived NK cell expansion and function by osteoclasts. Therefore, although the frequencies of NK cells are lower in the peripheral blood of hu-BLT mice, their function is similar to those obtained from human donors. Hu-BLT mice were implanted with undifferentiated MP2 tumors (FIG. 20A) and those differentiated with NK-supernatants as described before (FIG. 27A) in the pancreas, and their growth dynamics and overall effect on mice were studied. MP2 tumors grew rapidly and formed tumors in the pancreas, and mice exhibited all the signs of morbidity within 6-7 weeks, and upon sacrifice at week 7, they exhibited tumors which spanned the entire abdomen and enveloped the spleen, stomach, and a portion of intestines (FIG. 20B, panel a). When NK-differentiated MP2 tumors were implanted in mice, no tumors were seen, and mice did not exhibit any signs of morbidity (FIG. 20B, panel c). In the in vitro cell cultures, NK-differentiated MP2 tumors similar to patient derived PL12 differentiated tumors grew slower when compared to undifferentiated MP2 tumors. The proportions of huCD45+ cells in pancreas were significantly decreased in mice implanted with MP2 tumors (3.37%) when compared to control mice (7.46%) likely reflecting the increased tumor burden in these mice (FIG. 27B), however, those implanted with NK-differentiated MP2 tumors maintained higher proportions of huCD45+ cells (10.19%), and furthermore, the percentages of huCD3+ T cells within huCD45+ cells were much higher in MP2 implanted tumors (80%) when compared to either NK-differentiated MP2 tumor implanted mice (62%) or control mice (45%) (FIG. 20C and Fib. 27B).

### Example 13: Single Injection of NK Cells Inhibited Tumor Growth in Mice Implanted with MP2 Tumors

Mice implanted with MP2 tumors and injected with 1.5×10⁶ super-charged NK cells with potent cytotoxic and cytokine secretion capabilities (FIG. 20A) exhibited no or substantially smaller tumors, without the involvement of other organs or signs of morbidity (FIG. 20B, panel b). Due to increased morbidity and mortality in tumor bearing mice by fast growing tumors in 7 weeks after tumor implantation, we shortened the time of sacrifice to 4-5 weeks after tumor implantation to be able to study the pancreas and the dynamics of immune cell infiltration in the pancreas. Greater numbers of huCD45+ cells were seen in the single cells prepared from the dissociated pancreas of either NK-injected tumor-bearing mice (9.2%) or NK-differentiated tumor-implanted mice (10.19%) or in the healthy control mice (7.46%) when compared to those from tumor-bearing mice (3.37%) (Fib. 27B). In addition, increased percentages of huCD45+CD3+ T cells were seen in cells dissociated from the pancreas of MP2 implanted mice (FIG. 20C and FIG. 27B), whereas cells dissociated from the pancreas of tumor-bearing mice which received NK cells or from mice with implanted NK-differentiated tumors or from control healthy mice exhibited relatively lower percentages of huCD45-CD3+ T cells (FIG. 20C and FIG. 27B) and higher percentages of NK cells in the dissociated pancreas when compared to those from tumor-bearing mice (FIG. 20D). Likewise, greater than two-fold increase in huCD16+ NK cells within the cells dissociated from the pancreas were seen from either NK-injected tumor-bearing mice or from the pancreas of healthy control mice with no tumor implantation, when compared to those from tumor-bearing mice (FIG. 20D).

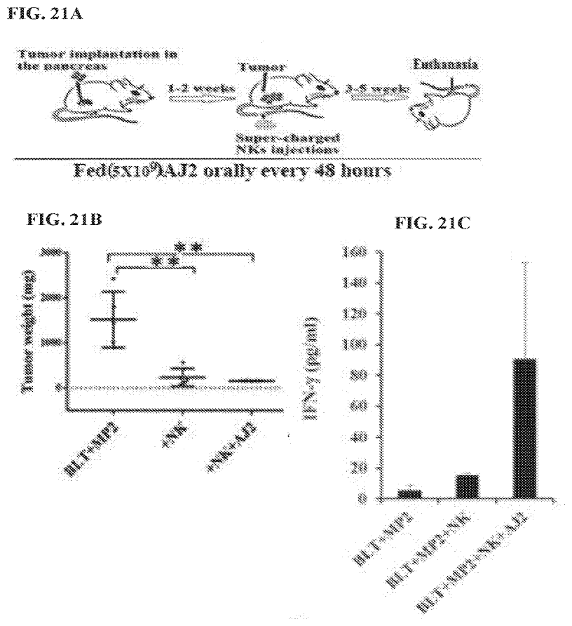

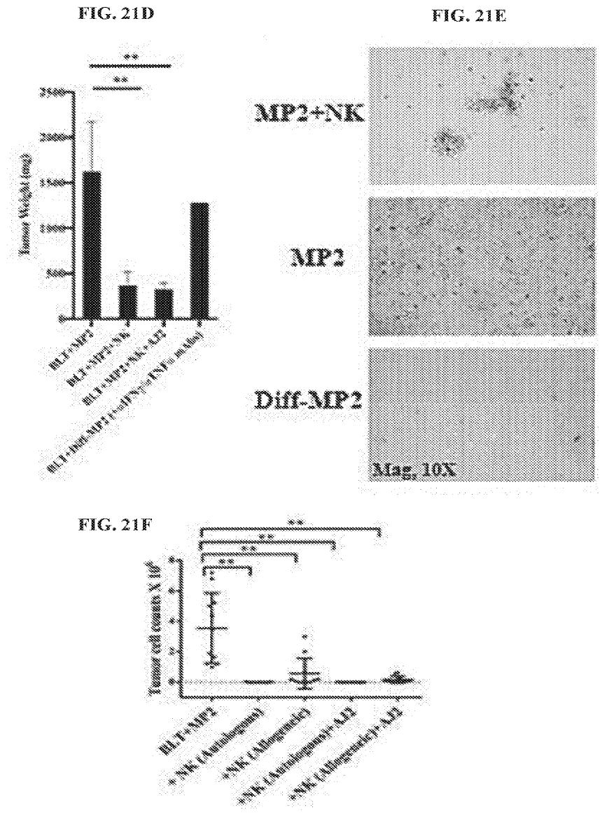

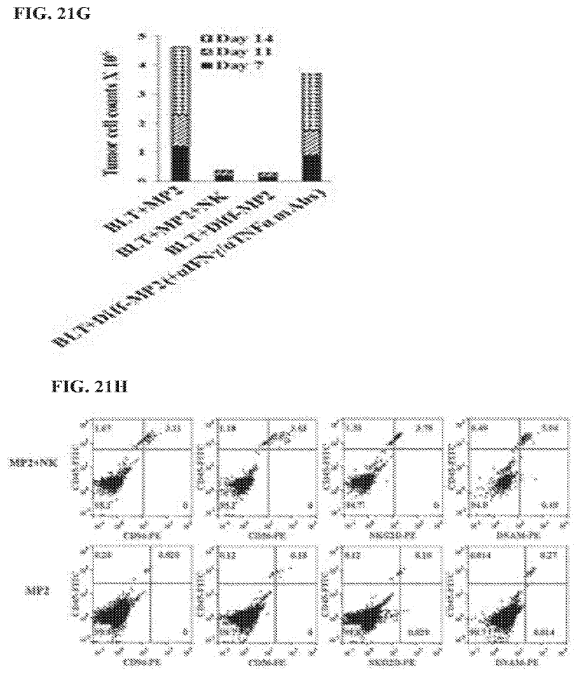

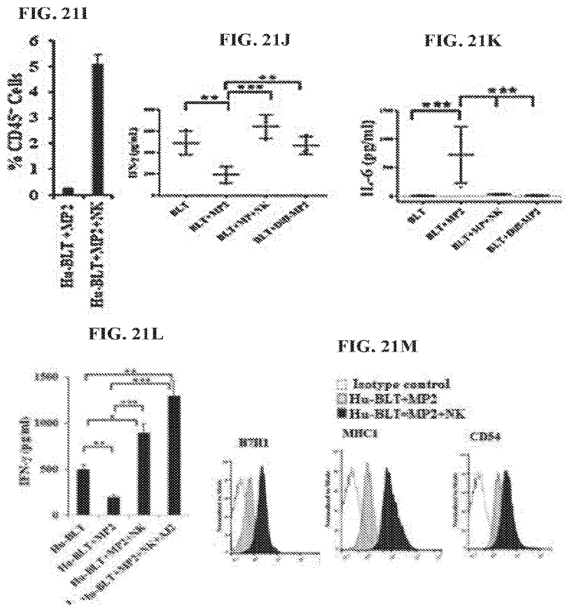

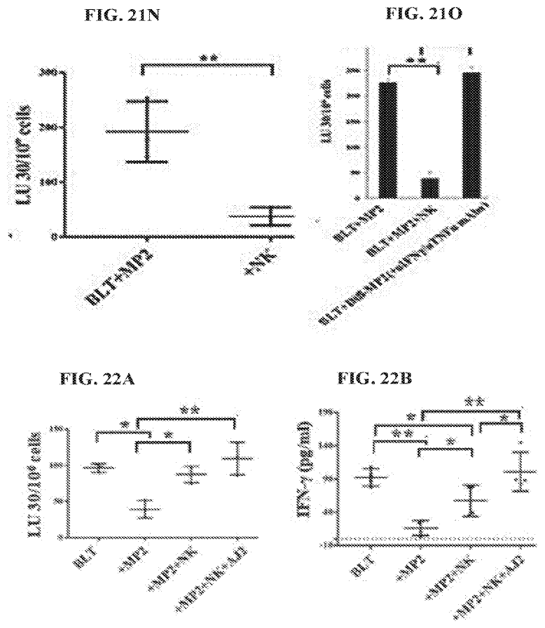

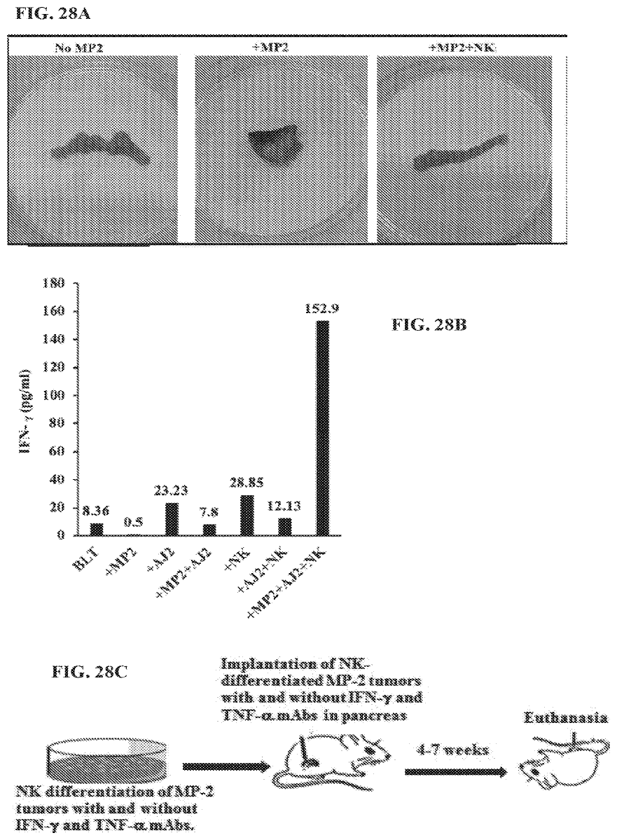

Unlike tumor-bearing mice, when mice were fed AJ2 1-2 weeks before tumor implantation and injected with allogeneic or autologous super-charged NK cells (FIG. 21A and FIG. 28A; see below), their tumor weights remained substantially less (FIG. 21B). No statistically significant differences in tumor weight could be observed between NK or NK injected/AJ2 fed mice, even though a slight decrease in the average tumor weight could be seen between the two groups (FIG. 21B). This is likely due to the significant decrease already seen with NK injection alone in tumor bearing mice. Indeed, sera from the peripheral blood of either NK-injected or NK injected/AJ2 fed tumor-bearing mice exhibited 2.73 and 4.8-fold more IFN-γ, respectively, when compared to tumor-bearing mice (FIG. 21C and FIG. 28B). Feeding AJ2 alone, or injecting super-charged NK cells in the absence of tumor implantation, or feeding AJ2 with implantation of tumors, or injecting super-charged NK cells and feeding AJ2 all increased the levels of IFN-γ in the serum of the hu-BLT mice moderately when compared to control mice in the absence of any treatments. These mice had much less IFN-γ in the sera when compared to those which were implanted with the tumor and fed with AJ2 and injected with super-charged NK cells (FIG. 28B). Mice with implantation of the tumor in the absence of any treatment had the least amount of IFN-γ in the sera (FIG. 21C and FIG. 28B). Similarly, mice implanted with NK-differentiated MP2 tumors (FIG. 27A) had minimal tumor weight (FIG. 21D), and blocking MP2 differentiation with anti-IFN-γ and anti-TNF-α antibodies (FIG. 21D and FIG. 28C) resulted in the inhibition of tumor differentiation and generation of tumors with higher tumor weights (FIG. 21D).

When pancreata were removed, dissociated and equal numbers of cells were cultured from tumor-bearing mice which did not receive NK injection, attached colonies of tumors could be seen in 24-48 h and they grew rapidly thereafter, whereas those injected with allogeneic NK cells or autologous NK cells (FIG. 21E and FIG. 21F) did not exhibit colonies initially, but a few were visible after day 5 or 6 and those colonies grew very slowly, and the numbers of tumors recovered remained substantially lower in comparison to those which did not receive NK injection (FIG. 21E and FIG. 21F). Similarly, in NK-differentiated tumors, when implanted in mice and their pancreas were dissociated after sacrifice, tumors did not grow or grew very few colonies at later days and their growth remained extremely slow (FIG. 21E and FIG. 21G), however, blocking differentiation with anti-IFN-γ and anti-TNF-α antibodies allowed attachment and growth of the tumors at 24-48 h with increased kinetics of growth (FIG. 21G). Tumor growth after dissociation and plating was less in mice fed with AJ2 and injected with NK cells in comparison to NK alone injected mice, and both were substantially less than those which only received implantation of the MP2 tumors (FIG. 21F). There was 18-22 fold more infiltrating huCD45+ immune cells in pancreas cultured from mice injected with tumors and NK cells in comparison to tumor-alone injected mice (FIG. 21H, FIG. 21I, and FIG. 27C). Greater percentages of infiltrating huCD45+ immune cells within the pancreas of NK injected tumor-bearing mice expressed CD94, and NKG2D surface receptors, whereas they expressed similar percentages of DNAM surface receptors when compared to tumor-bearing mice in the absence of NK injection (FIG. 21H, FIG. 27C, and FIG. 27D).

On average, a decrease in IFN-γ secretion from the pancreatic cell cultures could be observed in mice implanted with MP2 tumors, when compared to control mice with no tumors (FIG. 21J). Injection of NK cells into tumor-bearing mice restored IFN-γ secretion in pancreatic cell cultures and the levels exceeded those seen in the control mice with no tumors (FIG. 21J). Implantation of NK-differentiated MP2 tumors did not result in inhibition of IFN-γ in pancreatic cell cultures, and the amounts were comparable to those obtained from control mice with no tumors (FIG. 21J). In contrast, IL-6 secretions were the highest in pancreatic cell cultures from tumor-bearing mice, and they were substantially lower in all other groups of mice (FIG. 21K). Although slight differences could be seen between NK alone injected or NK-injected and AJ2 fed mice in terms of tumor weight/tumor growth in pancreas, there was, on average, higher secretion of IFN-γ by NK injected and AJ2 fed pancreatic cell cultures (FIG. 21L).

MP2 tumors cultured from the pancreas of NK-injected mice exhibited increased expression of B7H1 (PD-L1), MHC-class I and CD54 when compared to tumor-bearing mice without NK injection (FIG. 21M). Moreover, similar to our in vitro experiments, MP2 tumors cultured from the pancreas of NK-injected mice exhibited decreased sensitivity to NK cell-mediated lysis, whereas those cultured from tumor-bearing mice without NK injection exhibited increased susceptibility (FIG. 21N and FIG. 21O)). The addition of antibodies against IFN-γ and TNF-α during NK cell mediated differentiation of pancreatic tumors was found to restore the tumors' susceptibility to NK cell-mediated cytotoxicity (FIG. 21O). Since we were unable to recover enough tumors after implantation of NK-differentiated tumors in the pancreas; we were unable to run cytotoxicity assay.

### Example 14: Suppression of NK Cell Cytotoxicity and Decreased Secretion of IFN-γ in Tumor-Bearing Mice within all Tissue Compartments: Restoration by Super-Charged NK Cells

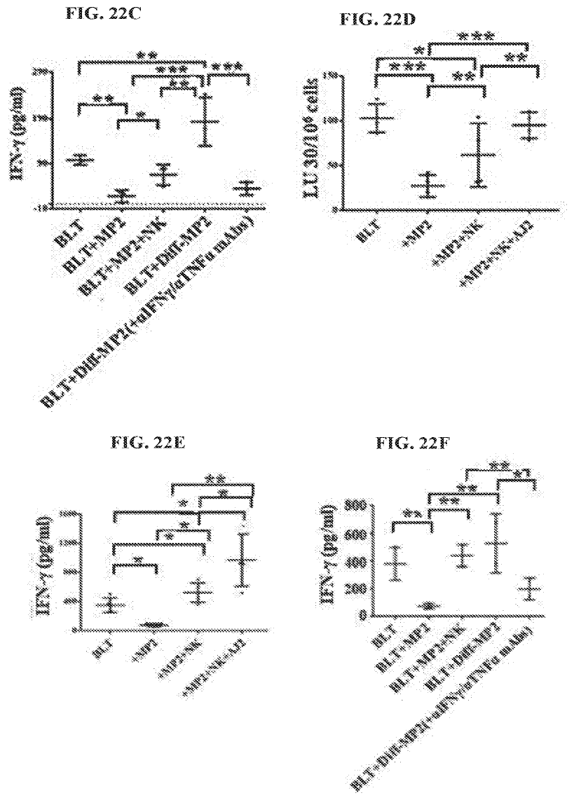

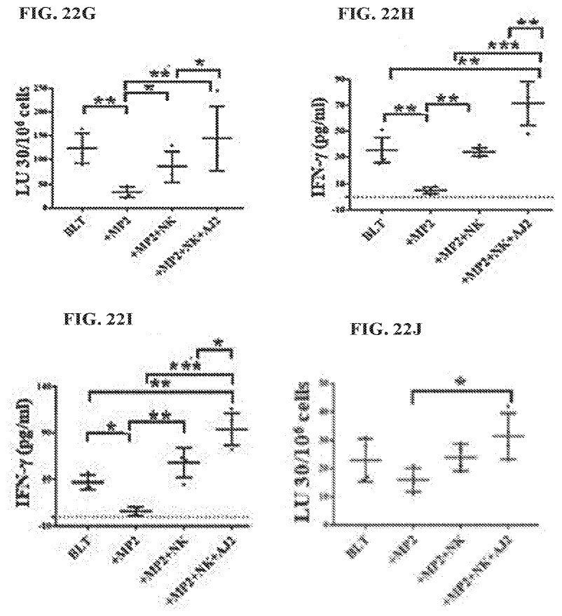

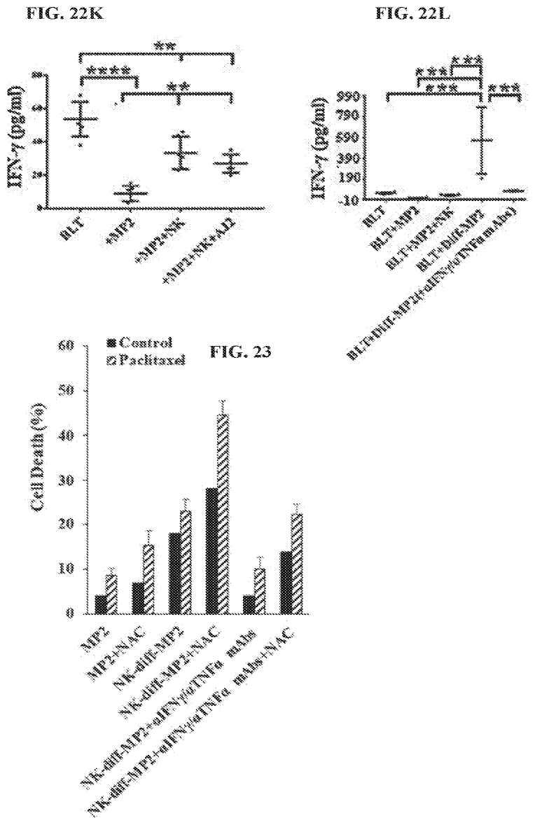

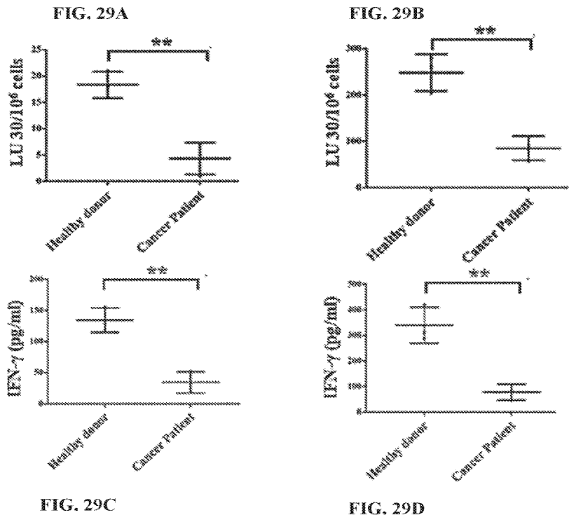

PBMCs from tumor-bearing mice (FIG. 22A), which were similar to PBMCs (FIG. 29A and FIG. 29C) and NK cells (FIG. 29B and FIG. 29D) from pancreatic cancer patients, had significantly lower NK cell-mediated cytotoxicity and exhibited decreased IFN-γ secretion, when compared to those from healthy mice or humans, respectively. When PBMCs (FIG. 22A-22C), splenocytes (FIGS. 22D and 22F), enriched-NK cells from splenocytes (FIG. 22G and FIG. 22H), huCD3+ T cells from splenocytes (FIG. 22I), and BM-derived immune cells (FIG. 22J-FIG. 22L) were assessed for NK cytotoxicity and/or secretion of IFN-γ, tumor-bearing mice had much lower cytotoxicity and/or secretion of IFN-γ in cells obtained from all tissue compartments, in comparison to those obtained from control mice without tumor, or tumor-bearing mice injected with NK cells, or those implanted with NK-differentiated tumors (FIG. 22). Blocking NK differentiation of the tumors by anti-IFN-γ and anti-TNF-α antibodies resulted in a similar magnitude of IFN-v secretion to those obtained from undifferentiated tumors in all tissue compartments tested (FIG. 22C,F,L).

Similar to those seen with the pancreatic tumors, implantation of oral stem-like tumors in the oral cavity of hu-BLT mice resulted in similar profiles of cytotoxicity and secretion of IFN-γ from PBMCs isolated from oral tumor bearing mice in the presence and absence of NK injection.

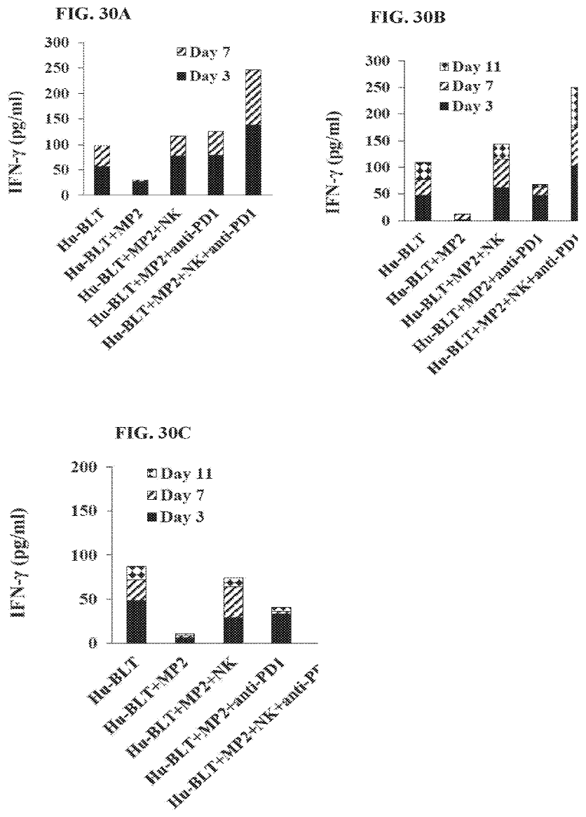

IV injection of anti-PD1 in combination with NK cells elevated secretion of IFN-γ in different tissue compartments tested (FIG. 30A-30C). Anti-PD1 antibody injection in the absence of NK cells in tumor-bearing mice increased secretion of IFN-γ in the tissues (FIG. 30A-30C).

### Example 15: Paclitaxel Induce Cell Death in NK-Differentiated MP2 Tumors Treated with/without N-Acetyl Cysteine (NAC)

Unlike MP2 tumors, treatment of well-differentiated PL12 and Capan tumors with paclitaxel (FIG. 4) exhibited higher induction of cell death. Similarly, when MP2 tumors were differentiated with NK-supernatants and treated with paclitaxel, higher induction of cell death was observed in MP2 tumors (FIG. 23). Inhibition of NK-mediated differentiation by the addition of antibodies to IFN-γ and TNF-α substantially decreased the cell death induced by paclitaxel (FIG. 23). As shown in (FIG. 23), the addition of NAC to MP2, PL12, and Capan increased paclitaxel mediated cell death. Similarly, the addition of NAC to NK-supernatant differentiated MP2 tumors increased cell death, and blocking differentiation with IFN-γ and TNF-α mAbs decreased paclitaxel mediated cell death (FIG. 23). The differentiation potential of cells by NAC was shown, and the addition of paclitaxel or cis-dichlorodiammineplatinum (CDDP or Cisplatin) to patient-derived differentiated oral squamous carcinoma cells (OSCCs) or NK-differentiated OSCSCs also mediated higher cell death, whereas minimal effects were seen on stem-like/poorly differentiated OSCSCs.

### Example 16: Monocytes or Osteoclasts from NK Injected Tumor Bearing Mice or NK-Differentiated Tumor Bearing Mice had Higher Capacity to Activate NK Cells

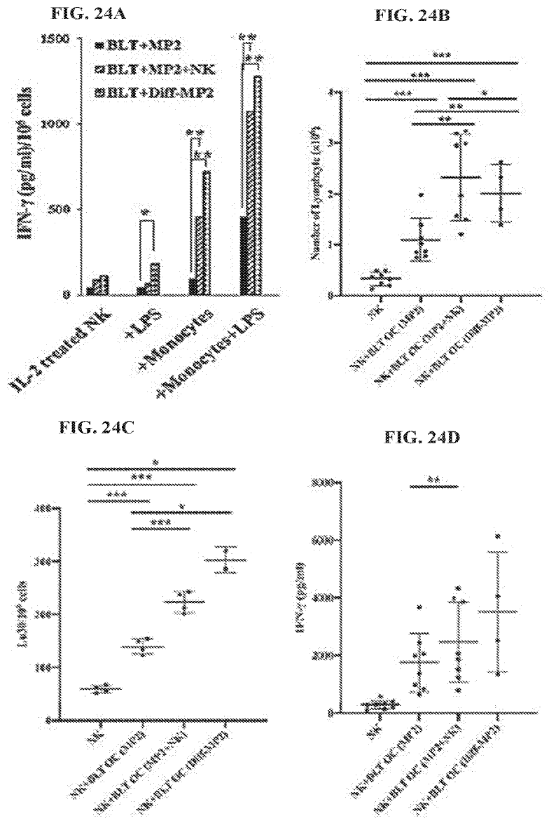

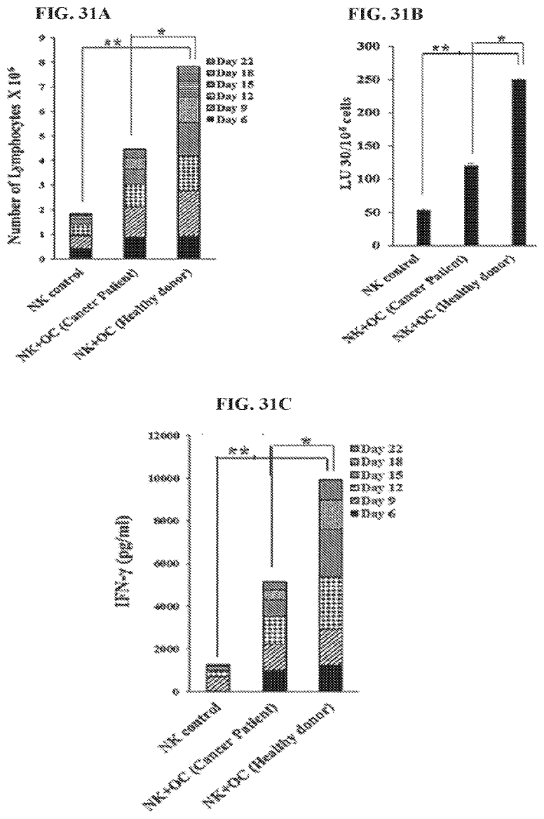

When NK cells were cultured in the presence of autologous monocytes from tumor-bearing mice injected with the NK cells or those implanted with NK-differentiated MP2 tumors, they demonstrated increased secretion of IFN-γ (FIG. 24A). Similarly, NK cells cultured with osteoclasts from tumor-bearing mice injected with NK cells or implanted with NK-differentiated tumors had significantly greater expansion and function of NK cells when compared to those from tumor-bearing mice in the absence of NK injection (FIG. 24B-D). Similar results to those seen with tumor bearing hu-BLT mice were also seen when osteoclasts from pancreatic-cancer patients were cultured with NK cells (FIG. 31A-C). Osteoclasts from cancer patients were less able to expand NK cells (FIG. 31A), or increase NK cell-mediated cytotoxicity (FIG. 31B) or increase NK cell-mediated secretion of IFN-γ (FIG. 31C) when compared to those from healthy donors. When examining the surface receptor expression on cancer-patient and healthy individuals' osteoclasts, decreased expression of MHC-class I, CD54, KLRG1, KIR2, and MICA/B could be seen on cancer patients' OCs as compared to healthy OCs (FIG. 31).

Finally, when identical amounts of IFN-γ from the supernatants of NK cells were used to differentiate OSCSC tumors, those from pancreatic cancer patients' NK cells were less effective in differentiating OSCSC tumors as compared to those from healthy donors' NK cells (FIG. 32). NK supernatants from patients elevated MHC-class I expression moderately (FIG. 32) and induced only 35% resistance of OSCSC tumors to NK-mediated cytotoxicity (FIG. 32), whereas NK supernatants from healthy individuals elevated MHC-class I substantially (FIG. 32) and induced 78% resistance of OSCSCs against NK-mediated cytotoxicity (FIG. 32). The rationale for using OSCSCs is because these cells are highly sensitive to IFN-γ mediated differentiation.

NK cells limit growth and expansion of CSCs/poorly differentiated pancreatic tumors by tumor lysis and differentiation. MP2 tumors, being poorly differentiated, form large tumors in NSG and hu-BLT mice, and have the ability to metastasize, whereas their NK-differentiated tumors or patient-derived well-differentiated tumors form very small tumors in the pancreas without metastatic potential. Indeed, the growth potential of MP2 tumors in in vitro cultures is found to be 10-15 fold, whereas those of the NK-differentiated counterparts are between 1.5-4 fold when the same numbers of tumors are cultured within the same time period, and no or slight cell death could be seen in the cultures of either undifferentiated MP2 tumors or those differentiated by the NK cells. The slower growth rates of well differentiated pancreatic tumors in comparison to MP2 tumors were also shown previously.

Patient-derived PL12 tumors or NK-differentiated tumors, although not killed by primary NK cells, were however, susceptible to chemo-drugs and were killed by paclitaxel (FIG. 4 and FIG. 23) as well as CDDP, whereas poorly differentiated tumors were resistant. Indeed, when NK-differentiation of MP2 tumors was inhibited by the combination of IFN-γ/TNF-α antibodies, tumors lost their sensitivity to chemotherapy and became susceptible to NK cell mediated cytotoxicity. Moreover, NAC, which is known to differentiate cells in addition to its other effects, increased paclitaxel mediated death of NK-differentiated MP2 and well-differentiated tumors (FIG. 4 and FIG. 23).

Both autologous and allogeneic osteoclasts were able to expand hu-BLT NK cells with hu-BLT osteoclasts having slightly higher NK expansion potential and higher levels of IFN-γ secretion (FIGS. 26E and 26F). Similarities in NK responses between hu-BLT and human NK cells to be expanded by their autologous osteoclasts, and secrete increased levels of IFN-γ and mediate augmented cytotoxicity partly provides the rationale for the use of this animal model as a surrogate model for the studies of human disease. Furthermore, similar defects in both tumor-bearing hu-BLT and cancer patients' NK cells were found.

NK-differentiated MP2 tumors did not grow in hu-BLT mice, and when tumor differentiation was prevented by using antibodies to IFN-γ and TNF-α, tumors grew substantially (FIG. 21G). In contrast, blocking IL-6 or IL-8 with antibodies was not able to influence differentiation of tumors by the NK cells. Immunotherapy with super-charged NK cells in the presence or absence of AJ2 feeding resulted in a significant inhibition of tumor growth in hu-BLT mice. The rationale for feeding AJ2 was to maintain and increase NK cell activation in vivo, since recent studies from our laboratory and those of the others have shown significant increases in NK cell function by probiotic bacteria.

Tumors grew slower in tumor-bearing mice injected with NK cells, and they were of differentiated phenotype, whereas those in the absence of NK injection grew rapidly and remained undifferentiated. Moreover, tumors cultured from NK-injected tumor-bearing hu-BLT mice contained about 18-22 fold more huCD45+ immune cells and secreted higher IFN-γ in the presence of lower IL-6 secretion, whereas those cultured from tumor-bearing mice in the absence of NK injection had lower infiltrating huCD45+ cells and secreted lower IFN-γ in the presence of much higher IL-6 secretion. The increased secretion of IFN-γ was observed not only in tumor tissues, but also in all tissues examined from tumor-bearing mice fed with AJ2 and injected with NK cells when compared to those of tumor-bearing mice. Increased IL-6 secretion is likely due to the growing tumors in tumor-bearing mice.

The single injection of super-charged NK cells in tumor-bearing mice resulted in an increased surface receptor expression of PD-L1, CD54, and MHC-class I on tumor cells exhibiting decreased tumor growth and the loss of susceptibility of tumor cells to NK cell-mediated cytotoxicity (FIG. 21L-FIG. 21O), potentially paving the road for their increased susceptibility to cytotoxic T lymphocyte (CTL) mediated killing due to increased MHC-class I expression. Increased percentages of T cells in the presence of decreased NK cells in the pancreas of tumor-bearing mice could be problematic for successful removal of undifferentiated tumors since these tumors are not eliminated by the T cells.

It should be emphasized that malignant tumors are not the only cells that are able to influence the function of NK cells within the tumor microenvironment. There are many other cells, including stromal cells such as tumor associated fibroblasts, fat cells, and other immune effectors within the pancreatic tumor microenvironment that could either increase the function of NK cells to drive differentiation of the tumors, or decrease their function resulting in the survival and expansion of stem-like/undifferentiated tumors depending on the early or late stages of cancer, respectively. In addition, at the later stages of cancer, many inhibitory effector cells such as T regulatory cells and MDSCs accumulate, and are therefore able to inhibit the function of NK cells resulting in the survival and expansion of cancer stem cells. Indeed, competent NK cells should be able to target and lyse MDSCs as they are able to lyse many different myeloid derived immune effectors.

In addition to releasing suppressive soluble factors into circulation, tumors can also suppress the function of NK cells by releasing various sized vesicles such as small, endosome-derived extracellular microvesicles of 30-100 nm exosomes which contain tumor proteins, mRNAs, and microRNAs, and larger-sized vesicles containing encapsulated cytosolic contents of 0.1 to 1 μm microparticles. Thus, tumors can profoundly inhibit the function of NK cells in cancer patients locally within the tumor microenvironment, and distantly within the peripheral blood and healthy tissues leading to irreversible damage of patients' NK cells.

Similar to cancer patients' monocytes and osteoclasts, those from tumor-bearing mice had much lower ability to expand autologous or allogeneic NK cells or increase their functional potential. More severe inhibition of NK cell expansion and function is seen when both NK and monocytes are from tumor-bearing mice due to the combined defects in both NK cells and monocytes. These experiments not only highlight similarities between the tumor-bearing hu-BLT mouse model and human cancers, but also indicate a severe functional deficiency in NK cell activating effectors in tumor-bearing hu-BLT mice similar to cancer patients. It is also important to note that the highest activation of NK cells from hu-BLT mice was achieved through the implantation of NK-differentiated tumors, suggesting that optimal differentiation of tumors can indeed promote and maintain intact monocyte/osteoclast function.

To understand the underlying mechanisms which govern inhibition of NK cell function by patient osteoclasts, it was determined herein the surface expression of osteoclasts from cancer patients in comparison to healthy donors' osteoclasts. The findings indicated that not only inhibitory MHC-class I expression is down-regulated, but also activating CD54, KLRG1, and MICA/B surface receptor expressions were decreased (FIG. 31), which indicates an overall decrease in NK ligand expression. Loss of activating ligands could clearly be a reason for decreased activation of NK cells; however, loss of inhibitory receptors provides a more complex picture. Loss of expression of both activating and inhibitory NK cell ligands was also seen on osteoclasts from KC mice with pancreatic KRAS mutation correlating with the loss of NK cell function and generation of pancreatic tumors.

Supernatants from patient's NK cells were less able to differentiate tumors, indicating that the function of secreted IFN-γ from patient NK cells is also severely compromised. Thus, pancreatic tumor induction and progression in patients is due to not only combined defects in NK expansion, decreased NK-cell mediated cytotoxicity and lower secretion of IFN-γ, and much lower ability of secreted IFN-γ to differentiate tumors, but also due to the defects in other subsets of immune cells which support NK cell expansion and function.

The studies presented herein indicate that immunotherapy by super-charged NK cells in the presence of AJ2 oral supplementation not only restores immune function in cancer patients by delaying or curtailing the growth potential of poorly-differentiated/stem-like pancreatic tumors, but also by expanding and activating CD8+ T cells. This not only allows NK-expanded CD8+ T cells to target NK-differentiated tumors, but, more importantly, they add to the pool of differentiated tumors since NK-expanded CD8+ T cells can also produce IFN-γ and TNF-α upon activation. NK and CD8+ T cell-differentiated tumors can also be targeted by radiotherapeutic and/or chemotherapeutic strategies. Although the role of NK cells in targeting metastatic tumors has been speculated for a long time, the mechanisms underlying the clearance of such tumors have not been clearly delineated. Previous works have focused on the killing ability of NK cells. However, the study presented herein demonstrates that both lysis and differentiation of tumors by the NK cells are important mechanisms by which NK cells are capable of preventing the induction and progression of tumors.

The studies presented herein indicated that an intact immune system is required for the elimination of tumors. However, tumors have been shown to cause immune suppression, in particular NK suppression, and this defect occurs in NK cells at many levels. NK cells from both cancer patients and humanized mice implanted with tumor lose their ability to kill and differentiate tumors. The inability of NK cells to curtail tumor growth through increased lysis and differentiation of tumors is a profound deficiency which will require significant intervention. Such intervention could be through the administration of super-charged NK cells, as we have seen in hu-BLT mice implanted with poorly differentiated pancreatic tumors.

### Example 17: Correlation Between NK Cell Cytotoxicity and the Stage of Differentiation of MP2 and PL-12 Pancreatic Tumors and the Role of rhTNF-α and rhIFN-γ in Induction of Differentiation and Resistance of MP2 Cells to NK Cell Mediated Cytotoxicity

As demonstrated herein, the stage of differentiation of the tumors was correlated with sensitivity to NK cell mediated cytotoxicity in pancreatic tumors. The highest susceptibility to NK cell mediated cytotoxicity was seen with undifferentiated MP2 tumors whereas the well differentiated PL-12 tumors demonstrated the lowest sensitivity to NK mediated lysis (FIG. 25A). Stem-like/undifferentiated MP2 and well differentiated Capan pancreatic tumor cells were treated with rhTNF-α and rhIFN-γ and their susceptibility to NK cell mediated lysis was assessed in a standard 4-hour ⁵¹Cr release assay. As shown in FIG. 25B, the combination of rhTNF-α and rhIFN-γ were able to upregulate CD54, MHC-1 and B7H1 and down modulate CD44 in MP-2 tumors. Both rhTNF-α and rhIFN-γ were able to increase surface expression of CD54 and MHC-class I, however, only rhIFN-γ was able to upregulate B7H1 (FIG. 25B). The addition of rhTNF-α to MP2 was able to induce moderate resistance against NK cell mediated cytotoxicity whereas rhIFN-γ induced significant resistance (FIG. 25C). As expected there was less lysis of Capan tumors by the NK cells and treatment with rhIFN-γ and rhTNF-α induced moderate resistance in these cells (FIG. 25C).

### Example 18: Reconstitution of Human Immune System in Hu-BLT Mice and Decreased Frequencies of NK Cells in Hu-BLT Mice as Compared to Humans

Hu-BLT mice that were reconstituted with the human immune system, exhibited greater than 90% reconstitution with huCD45+ immune cells in different tissue compartments (FIG. 26A-FIG. 26B). Similar to humans in which a range of frequencies can be seen in peripheral blood NK cells between donors, there are also variable percentages of NK cells in peripheral blood of hu-BLT mice reconstituted with different donor immune cells. Based on the number of hu-BLT mice tested so far, on average there is a lower percentage of NK cells in peripheral blood of hu-BLT mice as compared to human donor peripheral blood (FIG. 26C). Similar percentages of T cell subsets between human and hu-BLT mice in peripheral blood were found (FIG. 26C).

### Example 19: Frequencies of Immune Subsets in the Pancreas

The majority of infiltrating human immune cells in the pancreas were CD3+ T (54%) and B cells (43.3%), with CD8+ T cells constituting the larger proportions of the T cells (approximately 80%) than CD4+ T cells (approximately 20%) (FIG. 26D). NK and CD14+ cells constituted minor subpopulations of immune cells in the pancreas of healthy hu-BLT mice (FIG. 26D).

### Example 20: Materials and Methods for Examples 21-28

### Cell Lines, Reagents, and Antibodies

Oral squamous carcinoma stem cells (OSCSCs) were isolated from patients with tongue tumors at UCLA. OSCSCs were cultured in RPMI 1640 (Life Technologies, CA, USA) supplemented with 10% fetal bovine serum (FBS) (Gemini Bio-Product, CA, USA). RPMI 1640 supplemented with 10% FBS was used to culture human NK (cells, human T cells, and hu-BLT mice BM, spleen and PBMCs. Alpha-MEM (Life Technologies, CA, USA) supplemented with 10% FBS was used for osteoclast (OCs) and dendritic cell (DCs) cultures. M-CSF, anti-CD16 mAb, and flow cytometric antibodies were purchased from Biolegend, CA, USA. RANKL, GM-CSF, and IL-4 were purchased from PeproTech, NJ, USA, and recombinant human IL-2 was obtained from Hoffman La Roche (NJ, USA). Human anti-CD3/CD28 was purchased from Stem Cell Technologies, Vancouver, Canada. Probiotic bacteria, AJ2 is a combination of eight different strains of gram-positive probiotic bacteria (Streptococcus thermophiles, Bifidobacterium longum, Bifidobacterium breve, Bifidobacterium infantis, Lactobacillus acidophilus, Lactobacillus plantarum, Lactobacillus casei, and Lactobacillus bulgaricus) elected for their superior ability to induce optimal secretion of both pro-inflammatory and anti-inflammatory cytokines in NK cells. RPMI 1640 supplemented with 10% FBS was used to re-suspend AJ2. Human ELISA kits for IFN-γ were purchased from Biolegend (San Diego, Calif.). Phosphate buffered saline (PBS) and bovine serum albumin (BSA) were purchased from Life Technologies, CA, USA. Matrigel was purchased from Corning, N.Y., USA.

### Purification of Human NK Cells, T Cells and Monocytes

Written informed consents approved by the UCLA Institutional Review Board (IRB) were obtained from healthy donors and cancer patients (Table 1), and all procedures were approved by the UCLA-IRB. Peripheral blood mononuclear cells (PBMCs) were isolated from peripheral blood as described before. Briefly, PBMCs were obtained after Ficoll-hypaque centrifugation and were used to isolate NK cells, T cells, CD4+ T cells, CD8+ T cells, and monocytes using the EasySep® Human NK cell, EasySep® Human T cell, EasySep® Human CD4 T, and EasySep® Human CD8 T cell, EasySep® Human monocytes enrichments kits, respectively, purchased from Stem Cell Technologies (Vancouver, BC, Canada). Isolated NK cells, T cells, CD4+ T cells, CD8+ T cells, and monocytes were stained with anti-CD16, anti-CD3, anti-CD4, anti-CD8, anti-CD14 antibodies, respectively, to measure the cell purity using flow cytometric analysis.

### Generation of Human OCs and DCs

To generate OCs, monocytes were cultured in alpha-MEM media supplemented with M-CSF (25 ng/mL) and RANKL (25 ng/mL) for 21 days, media was replenished every three days. Monocytes were cultured in alpha-MEM media supplemented with GM-CSF (150 ng/mL) and IL-4 (50 ng/mL) for 7 days to generate DCs.

### Sonication of Probiotic Bacteria (AJ2)

AJ2 bacteria were weighed and re-suspended in RPMI 1640 medium containing 10% FBS at a concentration of 10 mg/ml. The bacteria were thoroughly vortexed, then sonicated on ice for 15 seconds at 6 to 8 amplitudes, sonicated samples were then incubated for 30 seconds on ice, cycle was repeated for five rounds. After every five rounds of sonication, we checked each sample under the microscope until at least 80% of bacterial walls were lysed. It was determined that approximately 20 rounds of sonication/incubation on ice were necessary to achieve complete sonication. Finally, the sonicated AJ2 (sAJ2) were aliquoted and stored at −80° C. until use.

### Expansion of Human NK Cells and Human T Cells

Human purified NK cells were activated with rh-IL-2 (1000 U/ml) and anti-CD16 mAb (3 μg/ml) for 18-20 hours before they were co-cultured with feeder cells (OCs or DCs) and sAJ2 (OCs:NK:sAJ2 or DCs:NK:sAJ2; 1:2:4) in RPMI 1640 medium containing 10% FBS. The medium was refreshed every three days with RPMI containing rh-IL-2 (1500 U/ml). Purified human T cells were activated with rh-IL-2 (100 U/ml) and anti-CD³ (1 μg/ml)/anti-CD28 (3 μg/ml) for 18-20 hours before they were co-cultured with OCs or DCs and sAJ2 (OCs:T:sAJ2 or DCs:T:sAJ2; 1:2:4) in RPMI 1640 medium containing 10% FBS. The culture media was refreshed with rh-IL-2 (150 U/ml) every three days.

### Enzyme-Linked Immunosorbent Assays (ELISAs) and Multiplex Cytokine Assay

Single ELISAs and multiplex assays were performed as previously described. To analyze and obtain the cytokine and chemokine concentration, a standard curve was generated by either two- or three-fold dilution of recombinant cytokines provided by the manufacturer. For multiple cytokine array, the levels of cytokines and chemokines were examined by multiplex assay, which was conducted as described in the manufacturer's protocol for each specified kit. Analysis was performed using a Luminex multiplex instrument (MAGPIX, Millipore, Billerica, Mass.), and data was analyzed using the proprietary software (xPONENT 4.2, Millipore, Billerica, Mass.).

### ⁵¹Cr Release Cytotoxicity Assay

The ⁵¹Cr release assay was performed as described previously. Briefly, different numbers of effector cells were incubated with ⁵¹Cr-labeled target cells. After a 4-hour incubation period, the supernatants were harvested from each sample and the released radioactivity was counted using the gamma counter. The percentage specific cytotoxicity was calculated as follows:

% Cytotoxicity=Experimental cpm−spontaneous cpm/Total cpm−spontaneous cpm

LU 30/10⁶ is calculated by using the inverse of the number of effector cells needed to lyse 30% of tumor target cells×100.

### Surface Staining Assay

For surface staining, the cells were washed twice using ice-cold PBS+1% BSA. Predetermined optimal concentrations of specific human monoclonal antibodies were added to 1×10⁴ cells in 50 μl of cold PBS+1% BSA, and were incubated on ice for 30 min. Thereafter cells were washed in cold PBS+1% BSA and brought to 500 μl with PBS+1% BSA. Flow cytometric analysis was performed using Beckman Coulter Epics XL cytometer (Brea, Calif.), and results were analyzed in the FlowJo vX software (Ashland, Oreg.).

### Tumor Implantation in Hu-BLT Mice

Animal research was performed under the written approval of the UCLA Animal Research Committee (ARC) in accordance with all federal, state, and local guidelines. Combined immunodeficient NOD.CB17-Prkdcscid/J and NOD.Cg-Prkdcscid Il2rgtm1Wjl/SzJ (NSG lacking T, B, and NK cells) were purchased from Jackson Laboratory. Humanized-BLT (hu-BLT; human bone marrow/liver/thymus) mice were prepared on NSG background as previously described. To establish orthotopic tumors, mice were first anesthetized with isoflurane in combination with oxygen, and 1×10⁶ human OSCSC tumor cells suspended in 10 l HC Matrigel were then injected directly into the floor of their mouths. One to two weeks after tumor implantation mice received 1.5×10⁶ OC-expanded NK cells via tail vein injection (FIG. 14A). Four to five later, mice were euthanized when signs of morbidity were evident and bone marrow, spleen, and peripheral blood were harvested.

### Cell Isolation and Cell Cultures of Hu-BL T Mice BM, Spleen, and Peripheral Blood

To obtain single-cell suspensions from BM, femurs were cut at both ends and flushed through using RPMI 1640 media; afterwards, BM cells were filtered through a 40 μm cell strainer. To obtain single-cell suspensions from spleen, the spleens were minced, and the samples were filtered through a 40 μm cell strainer and centrifuged at 1500 rpm for 5 minutes at 4° C. The pellet was re-suspended in ACK buffer for 2-5 mins to remove the red blood cells followed by re-suspension in RPMI media and centrifugation at 1500 rpm for 5 minutes at 4° C. PBMCs were isolated from peripheral blood using Ficoll-Hypaque centrifugation of heparinized blood specimens. The buffy coats containing PBMCs were harvested, washed, and re-suspended in RPMI 1640 medium. Cells obtained from each tissue sample were treated with IL-2 (1000 U/ml) and cultured in RPII 1640 medium containing 10% FBS for 7 days.

Target cell visualization assay (TVA)

Target cells were incubated with TVA™ dye at 37° C. for 15 mins and then cultured with effector cells for 4 hours. Afterwards, the target cells were counted with ImmunoSpot® S6 universal analyzer/software (Cellular Technology Limited, OH, USA) at 525 nm emission wavelengths. The percentage specific cytotoxicity was calculated as follows:

$$\% Cytotoxicity = \frac{Experimental cpm - spontaneous cpm}{Total cpm - spontaneous cpm}$$

LU 30/107 is calculated by using the inverse of the number of effector cells needed to lyse 30% of tumor target cells×100.

### Statistical Analyses

All statistical analyses were performed using the GraphPad Prism-8 software. An unpaired or paired, two-tailed student's t-test was performed for the statistical analysis for experiments with two groups. One-way ANOVA with a Bonferroni post-test was used to compare different groups for experiments with more than two groups. (n) denotes the number of human donors or mice for each experimental condition. Duplicate or triplicate samples were used in the in vitro studies for assessment. The following symbols represent the levels of statistical significance within each analysis: ***(p value <0.001), **(p value 0.001-0.01), *(p value 0.01-0.05).

### Example 21: Decreased Numbers and Suppression of Cytotoxicity and Secretion of IFN-γ by NK Cells in Cancer Patients

The numbers of PBMCs were significantly lower in the peripheral blood of cancer patients when compared to healthy individuals when identical amounts of blood was used to isolate PBMCs (FIG. 11A). Higher percentages of CD16+CD56+, CD14+, and CD11b+, and lower percentages of CD3+ and CD19+ cells were obtained within PBMCs of cancer patients when compared to healthy individuals (FIG. 11B). Cancer patients' NK cells secreted significantly lower amounts of IFN-γ (FIGS. 11C and 11E) and mediated lower cytotoxicity (FIG. 11D). In addition to IFN-γ, cancer patients' NK cells also secreted significantly lower levels of other cytokines (FIG. 11E). Decreased levels of cytokines were also seen in the sera of cancer patients when compared to those of healthy individuals (FIG. 11F). These findings indicated that cancer patients' peripheral blood contains fewer PBMCs and exhibit higher proportions of NK cells with substantially lower NK cell function in comparison to those of healthy individuals.

### Example 22: Allogeneic OC-Mediated Expansion, and Augmented Function of NK Cells from Cancer Patients is Greatly Suppressed when Compared to Those of Healthy Individuals

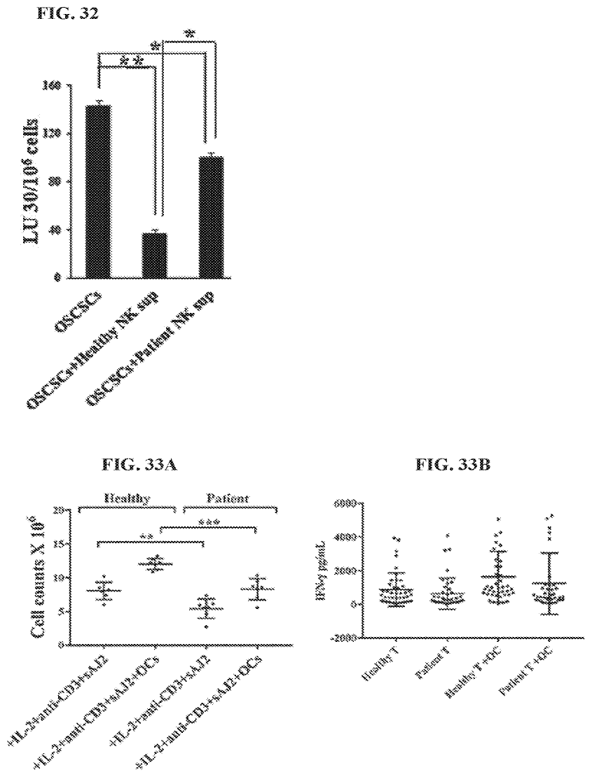

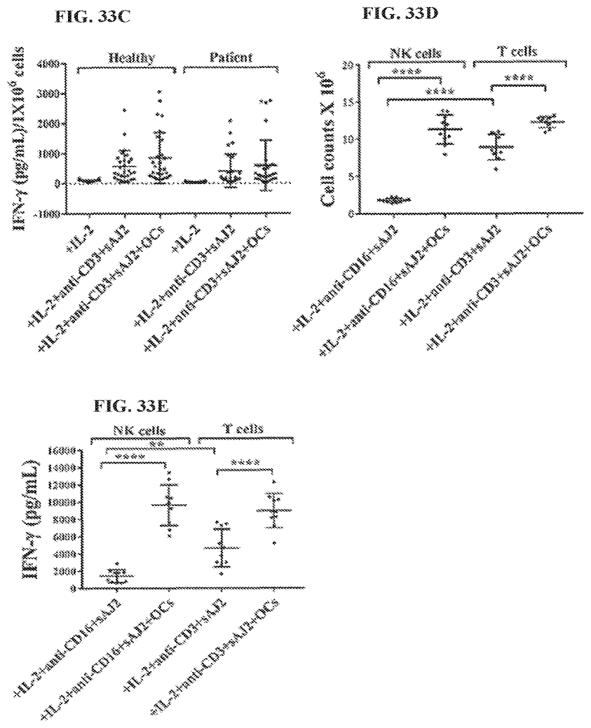

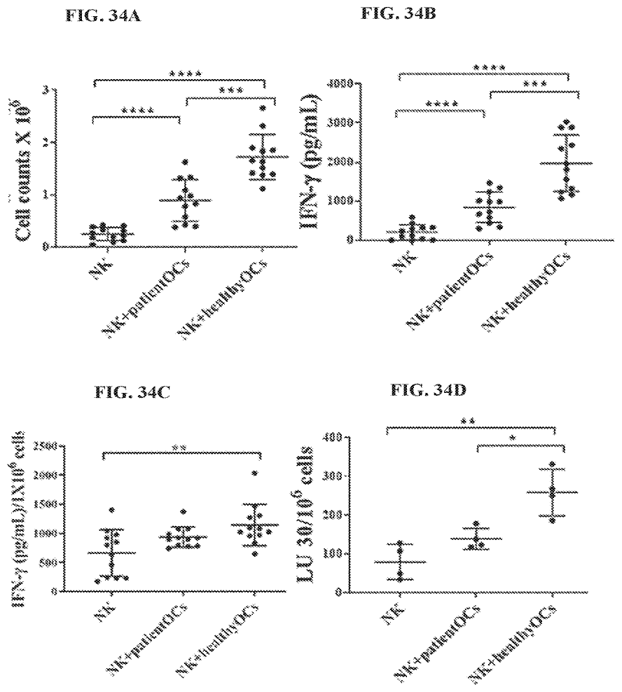

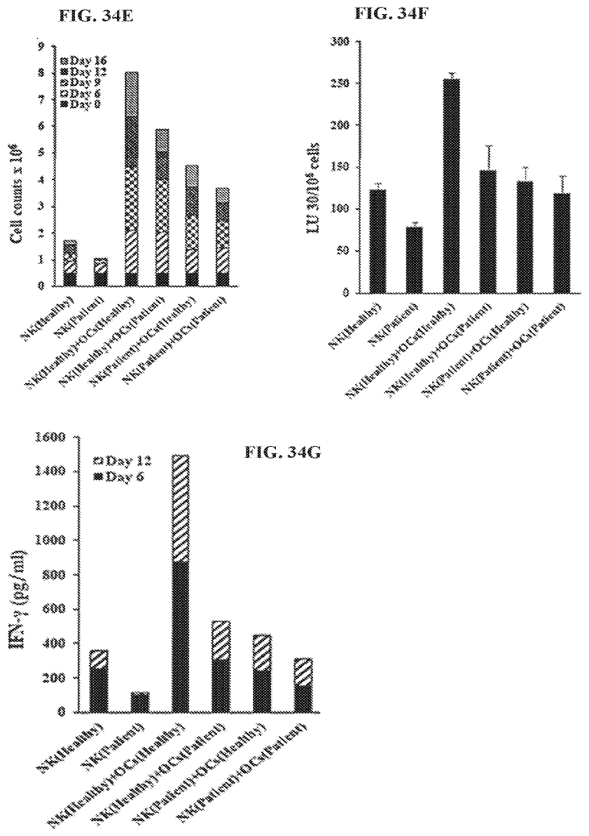

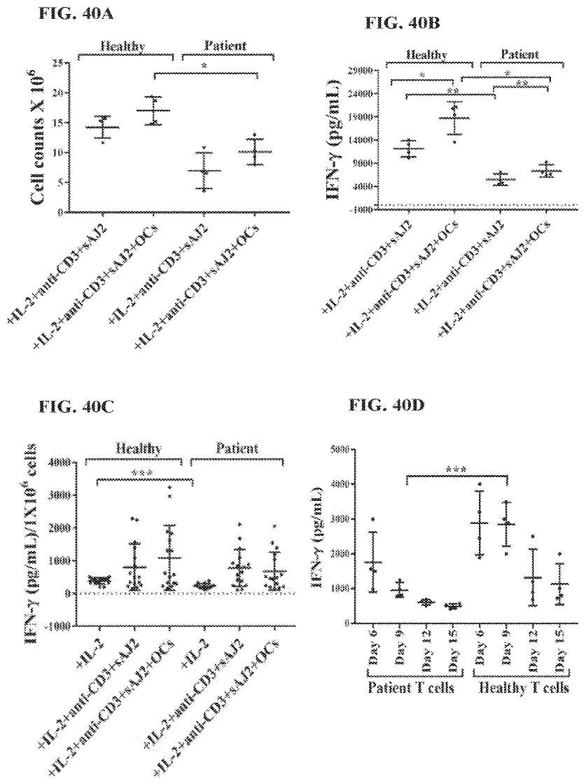

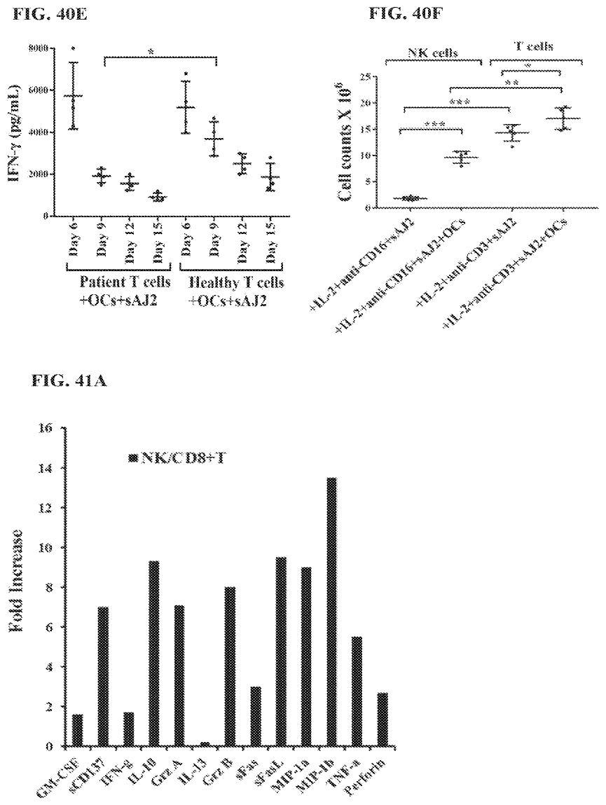

To determine the extent of NK and T cell expansion and function, we expanded NK and T cells of cancer patients and healthy individuals. Cancer patients' NK cells showed significantly decreased levels of expansion (FIGS. 12A and 34E), and expanded NK cells exhibited significantly lower cytotoxicity (FIGS. 12B and 34F), and IFN-γ secretion (FIGS. 12C, 12D, 42 and 34G) when compared to those of healthy individuals. Cancer patients' T cells exhibited similar decreases in expansion rate (FIGS. 33A and 40A) and IFN-γ secretion (FIGS. 33B-33C and 40B-40E).

To assess whether NK and T cells exhibit distinct expansion profiles, we cultured NK and T cells of healthy individuals in the presence and absence of OCs and found that T cells expanded faster than NK cells in the absence of OCs (FIGS. 33D and 40F). However, OCs induced 2.6-4.5 fold and 1.2-1.6 fold expansion in NK and T cells, respectively, when compared to those cultured in the absence of OCs (FIGS. 33D and 40F). These results indicated that OCs induce higher expansion of NK cells when compared to T cells. T cells secreted significantly higher IFN-γ secretion in the absence of OCs while IFN-γ secretion in both NK and T cells exhibited comparable increases in the presence of OCs (FIG. 33E).

### Example 23: Cancer Patients' OCs Induced Lower Cell Expansion, IFN-γ Secretion and Cytotoxicity in NK Cells when Compared to Healthy Individuals' OCs

To investigate the function of patients' OCs, we cultured the healthy individuals' NK cells with either autologous OCs or with patients' OCs (allogeneic). Patients' OCs were less capable of inducing NK cell expansion (FIGS. 2A and 2E), IFN-γ secretion (FIGS. 34B-34C and 34G), and NK cytotoxicity (FIGS. 34C and 34F). We next assessed the function of patient NK cells in the context of autologous OCs. Severe decreases in NK cell expansion, IFN-γ secretion, and cytotoxicity were observed when patients' NK cells were cultured with autologous OCs (FIGS. 34E-34G). These results indicated severe functional defects in both NK cells and OCs of cancer patients.

### Example 24: OC-Induced T Cell Mediated Expansion Increased CD8+ T Cells Moderately when Compared to OC-Induced NK Cell Mediated Expansion of CD8+ T Cells

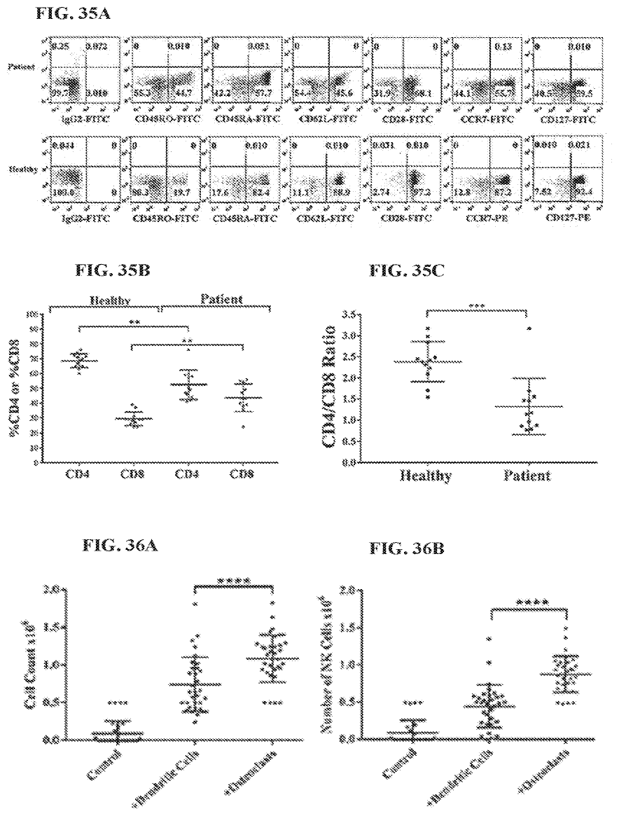

We next analyzed the surface phenotype of memory and naïve subpopulations of T cells, and observed increase in CD45RO+ cells (activated T cells) and decrease in CD45RA+ cells (naïve T cells) on cancer patients' T cells (FIG. 35A). We also noted reduced surface expressions of CD62L, CD28, CCR7, and CD127 on cancer patients' T cells (FIG. 35A). Moreover, percentages of CD4+ T cells were decreased with corresponding increase in the percentages of CD8+ T cells in cancer patients' PBMCs (FIG. 35B). Accordingly, we observed decreased CD4+/CD8+ T cell ratios in cancer patients' PBMCs, suggesting an overall increase in the CD8+ T cell subset in cancer patients when compared to healthy individuals (FIG. 35C).

We next cultured NK cells in the presence of healthy allogeneic OCs and determined the fractions of expanded CD4+ and CD8+ T cells within the expanded NK cells. No detectable T cells could be seen initially after NK cell purifications, however, after several rounds of NK cell expansions we were able to detect T cell expansion within the NK cells. The expanded T cells were primarily CD8+ T cells with no or very low levels of CD4+ T cells in cultures of expanded NK cells from both healthy individuals and cancer patients (FIG. 13A), and the relative CD4+/CD8+ T cell ratios remained similar between cancer patients and healthy individuals (FIG. 13B). However, it should be noted that the patients have higher percentages of CD8+ T cells than CD4+ T cells in their PBMCs when compared to those of healthy individuals (FIGS. 35B, 35C, 13A, and 13B).

When purified T cells were cultured with allogeneic healthy OCs, cancer patients but not healthy individuals exhibited higher percentages of CD8+ T cells with lower CD4+/CD8+ T cell ratios since the levels of CD8+ T cells were constitutively higher in cancer patients PBMCs in the absence of expansion (FIGS. 13A-13B). Also, it should be noted that for the sake of comparison we chose to activate T cells by IL-2 and anti-CD3/CD28 signaling since NK cells were activated by IL-2 and anti-CD16 mAbs before they were cultured with OCs. Thus, NK and T cells were pre-activated before their culture with OCs. We observed much higher percentages and numbers of CD8+ T cell expansion with no or very low remaining CD4+ T cells in OC-expanded NK cells, whereas OC-induced T cells expanded CD8+ T cells moderately with significant percentage of CD4+ T cells still remaining in the culture (FIGS. 13A-13B).

### Example 25: Increased NK Numbers and NK-Mediated Cytotoxicity by OC-Expansion in Comparison to DC-Expansion; OCs Preferentially Expand CD8+ T Cells Whereas DCs Preferentially Expand CD4+ T Cells in NK Cells Cultures

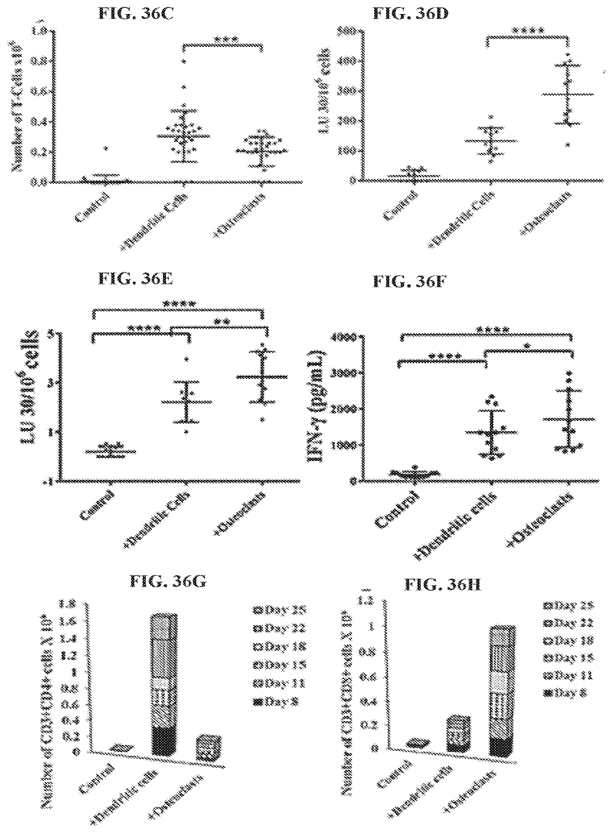

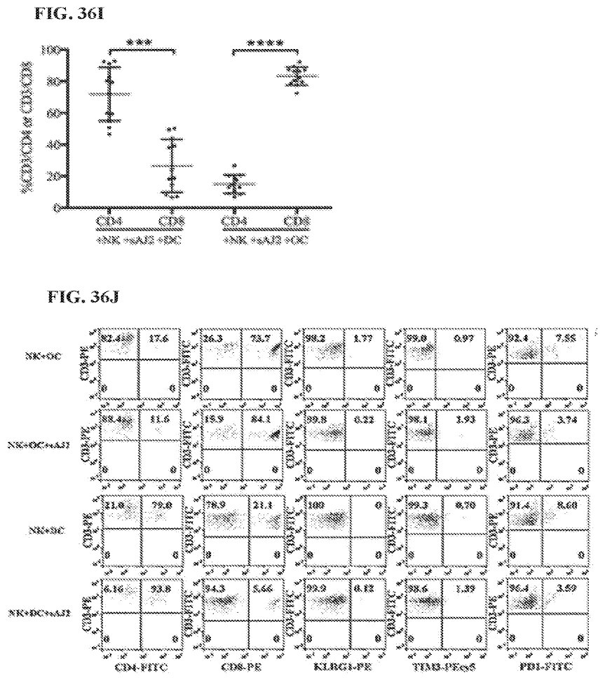

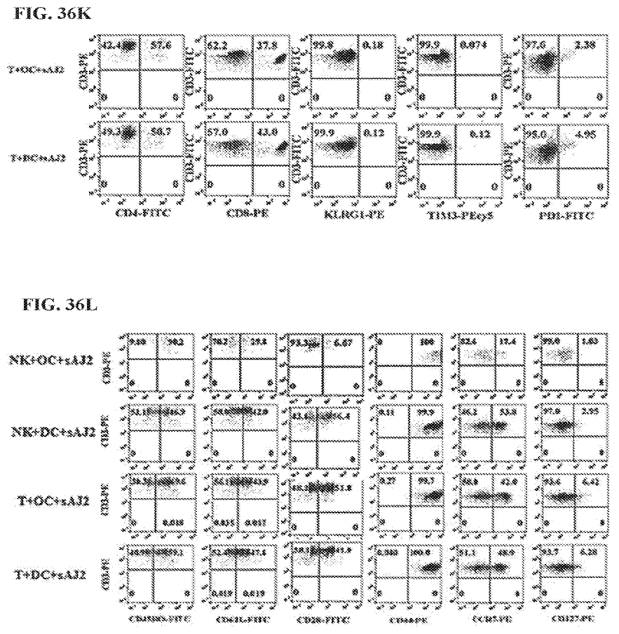

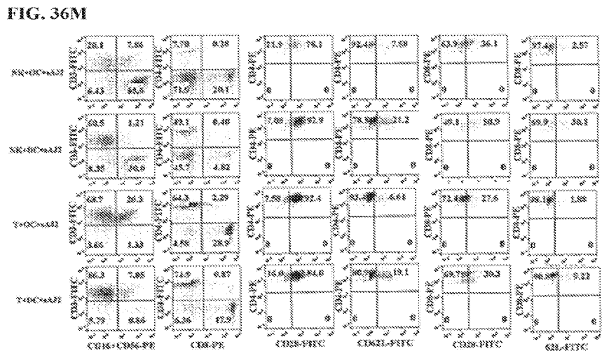

To assess whether the activation of NK cells by OCs vs. DCs differentially affects expansion profile and function, we cultured NK cells from healthy individuals either alone, with OCs, or with DCs. Significantly higher cell counts were observed in NK cells cultured with OCs in comparison to those cultured alone or with DCs (FIG. 36A). Next, we determined the subpopulations of CD16, and CD3 expressing cells within the NK cells cultured alone, or with OCs, or with DCs and counted the numbers of NK and T cells within total lymphocytes. Significantly higher NK cell counts (FIG. 36B) and lower T cell counts (FIG. 36C) were observed in the presence of OCs versus DCs. OC-expanded NK cells displayed significantly higher levels of cytotoxicity against oral squamous cancer stem-like cells (OSCSCs) (FIGS. 36D-36E). Additionally, NK cells cultured with OCs secreted significantly higher levels of IFN-γ than those cultured with DCs (FIG. 36F).

In addition, we characterized the subpopulations of T cells expanded within the NK cell cultures with OCs or DCs and found that DCs preferentially expanded CD4+ T cells (FIGS. 36G, 36I-36J and 36M) whereas OCs favored the expansion of CD8+ T cells (FIGS. 36H-36J and 36M). T cells expanded in NK cell cultures with OCs similar to those expanded by DCs did not express either killer cell lectin-like receptor G1 (KLRG1) or T cell immunoglobulin/mucin domain-containing protein 3 (TIM3), whereas they had similar levels of PD-1 (FIG. 36J). Thus, no significant levels of these check point inhibitors could be seen on T cells expanded by either OC- or DC-expanded NK cells (FIG. 36J). We also noticed slight differences in the expression levels of CD4, CD8, KLRG1, TIM3, and PD-1 in purified T cells cultured with OCs versus DCs in the absence of NK cells (FIG. 36K). T cells in NK+OC co-cultures expressed higher levels of CD45RO; lower levels of CD62L, CD28, CCR7, and CD127; and similar levels of CD44 when compared to NK+DCs co-cultures (FIGS. 36L-36M). When purified T cells were expanded in the presence of OCs, we observed slightly higher levels of CD45RO and CD28; lower levels of CD62L and CCR7; and similar levels of CD127 and CD44 when compared to those expanded in the presence of DCs (FIGS. 36L-36M).

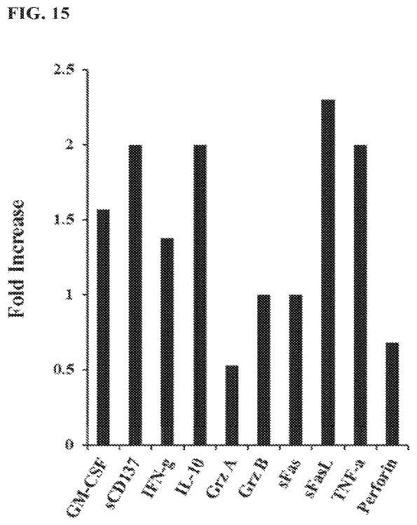

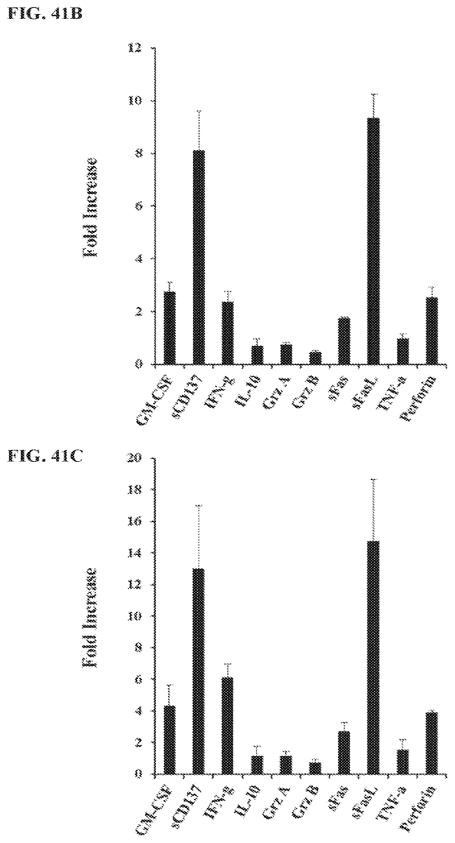

Correspondingly, higher levels of cytokines and chemokine secretions were seen in NK cells in comparison to T cells when both were cultured with OCs (FIGS. 41 and 15). NK cells secreted higher levels MIP-1a, MIP-1B, sCD137, FasL, GMCSF, IFN-γ, sFas, and perforin when compared to T cells (FIGS. 41A-41C). CD8+ T cells sorted out from OC-expanded NK cells culture secreted higher levels of GMCSF, sCD137, IFN-γ, FasL, IL-10, and TNF-α when compared to OC-expanded CD8+ T cells in the absence of NK cells (FIG. 15).

### Example 26: OCs Induce Higher Cell Expansion and IFN-γ Secretion in CD8+ T Cells than in CD4+ T Cells

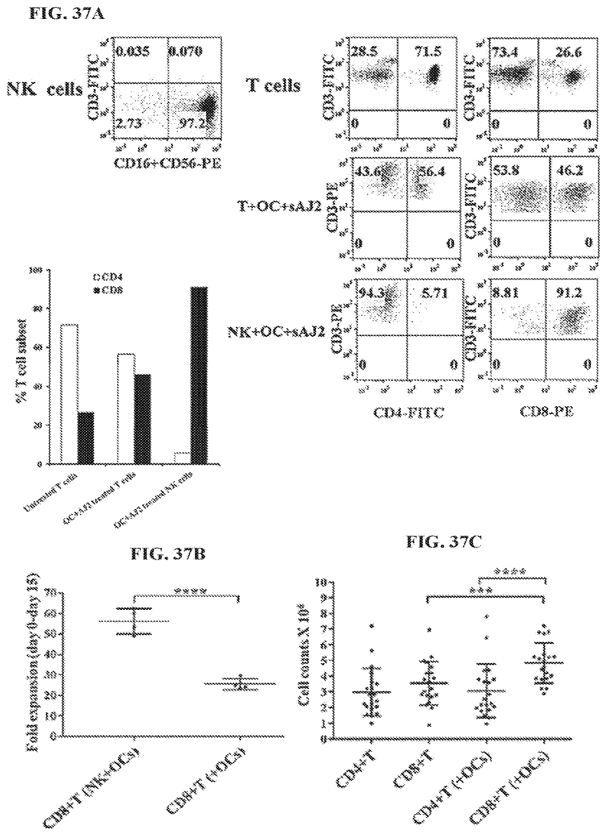

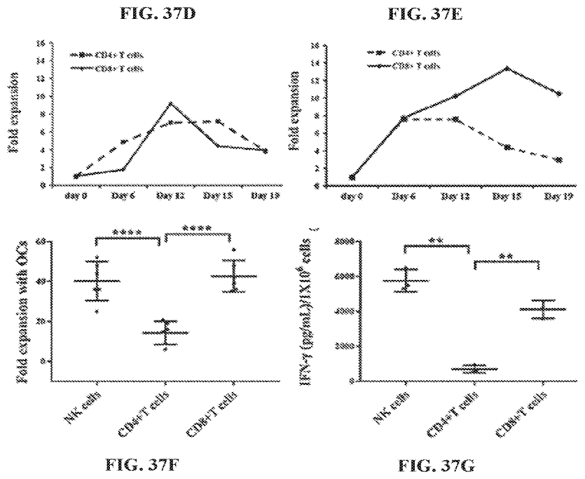

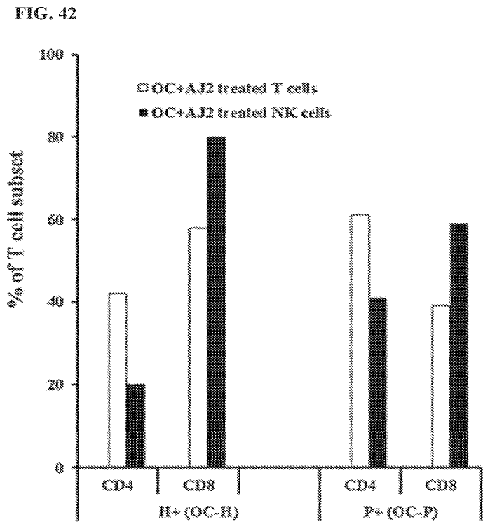

OCs were found to induce higher expansion of CD8+ T cells in NK cultures when compared to those with purified T cells (FIGS. 37A and 42) or purified CD8+ T cells (FIG. 37B). No significant differences in the degree of expansion could be seen when purified CD4+ and CD8+ T cells were treated with anti-CD3/CD28+IL-2 and cultured in the absence of OCs (FIGS. 37C-37D). However, in contrast to CD4+ T cells, a continuous rise in the fold expansion of CD8+ T cells could be seen when the cells were treated with anti-CD3/CD28+IL-2 and cultured with OCs (FIGS. 37C and 37E). Under the same experimental condition CD4+ T cell counts increase initially, but plateaued soon after, and then declined after day 12 of culture (FIG. 37E). We then compared the expansion and secretion of IFN-γ by the NK, CD4+ T, and CD8+ T cells after they were treated as described above and cultured with OCs. Higher cell expansion (FIG. 37F) and IFN-γ secretion (FIG. 37G) could be observed in NK and CD8+ T cells as compared to CD4+ T cells.

### Example 27: Increased CD8+ T Cells, IFN-γ Secretion, and Cytotoxicity in Various Tissue Compartments of Oral Tumor-Bearing Hu-BLT Mice in Response to NK Cell Immunotherapy

Hu-BLT mice were implanted with OSCSCs in the oral cavity and injected with OC-expanded NK cells with potent cytotoxic and cytokine secretion capabilities. After 4-5 weeks, the mice were sacrificed and tissues were harvested and dissociated in order to obtain single-cell suspensions for analysis (FIG. 14A). We observed increased proportions of CD3+CD8+ T cells in the BM (FIG. 14B), spleen (FIG. 14E), and peripheral blood (FIG. 14H) of tumor-bearing mice injected with OC-expanded NK cells when compared to tumor-bearing mice injected with vehicle only or healthy non-tumor bearing mice. NK cell immunotherapy also augmented the IFN-γ secretion and NK cell-mediated cytotoxicity in BM (FIGS. 14C and 14D), spleen (FIGS. 14F and 14G), and peripheral blood (FIGS. 141 and 14J) in tumor-bearing mice. Increased secretion of IFN-γ, IL-6, and ITAC and decreased secretion of IL-8 and GM-CSF were also seen in sera harvested from the peripheral blood of tumor-bearing mice injected with OC-expanded NK cells versus those injected with vehicle alone or non-tumor bearing mice injected with OC-expanded NK cells (FIG. 14K).

### Example 28: NK Cells Preferentially Lyse CD4+ T Cells when Compared to CD8+ T Cells

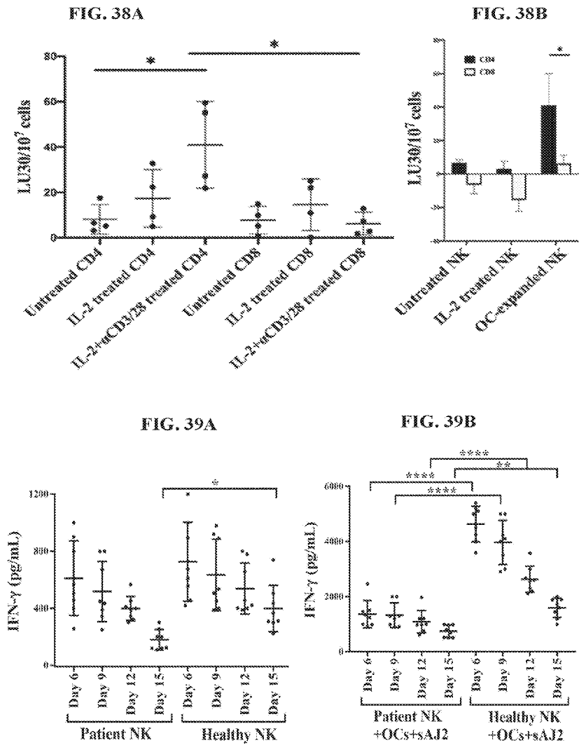

NK cell-mediated cytotoxicity against CD4+ and CD8+ T cells were assessed using TVA dye. OC-expanded NK cells preferentially lysed CD4+ T cells but not CD8+ T cells (FIG. 38A) and the levels were higher than those mediated by the IL-2 treated primary NK cells (FIG. 38B). Similarly, IL-2+anti-CD16mAb treated NK cells were able to lyse CD4+ T cells when compared to CD8+ T cells.

The dynamics of NK cell mediated regulation and activation of CD4+ and CD8+ T cells are presented herein. NK functional inactivation and loss of numbers occurs at both the pre-neoplastic and neoplastic stages of pancreatic cancer due to the effects of both the KRAS mutation and high fat calorie diet. It is demonstrated herein that patients with pancreatic cancer as well as a few other cancers have severely suppressed NK function. Both cytotoxicity and the ability to secrete IFN-γ are suppressed in patient NK cells. In addition, we also demonstrate that the percentages of NK, monocyte, and CD11b+ immune cells are increased in cancer patients, even though the total numbers of PBMCs are severely decreased. In addition, the percentages of CD3+ T cells and B cells are substantially decreased. Thus, although the percentages of NK cells are elevated in cancer patients, the function of NK cells are severely depressed, indicating a profound immunosuppression of NK cells from cancer patients. Even when NK cells were purified and expanded and super-charged by the use of OCs, the cells from cancer patients had much lower ability to expand and mediate cytotoxicity and secrete IFN-γ when compared to those expanded from healthy individuals. Thus, lower recovery of PBMCs from cancer patients could partly be due to the inability of different lymphocyte subsets such as NK cells to proliferate and expand when compared to those expanded from healthy individuals. Both primary and OC-expanded NK cells from cancer patients are defective in their function, therefore, the defects observed in patients' primary NK cells are dominant and are only moderately improved when these cells are expanded in the presence of allogeneic OCs. Expansion of patients' T cells as well as IFN-γ secretion from OC-expanded T cells are also decreased under different activation conditions (FIGS. 33A-33G, 40A-40C, AND 40E). Cancer patients' immune effectors demonstrated higher percentages of CD45RO and decreased percentages of CD62L surface expressions indicating the increased status of immune activation in vivo. This is also evident from the increased percentage of CD8+ T cells and decreased ratios of CD4/CD8 in patients (FIGS. 35B-35C).

Increased percentages of NK cells in cancer patients can be one reason why we see preferential increase in CD8+ T cells and lower ratios of CD4/CD8 T cells. Our studies indicate that NK cells are very important in the preferential expansion of CD8+ T cells. In particular, OCs are important in the expansion of NK cells. The majority of T cells expanded by the NK cells are CD8+ T cells, and similar profile of CD8+ T cell expansion by the NK cells is seen when NK cells are obtained from both healthy individuals and cancer patients indicating that NK cells are indispensable for the expansion of CD8+ T cells. Although OCs have some effect on the decreased ratios of CD4+ to CD8+ T cells in both healthy individuals and cancer patients T cells, the ratios are substantially decreased in the presence of NK cells indicating higher selection and expansion of CD8+ T cells and loss of CD4+ T cells by the expanded NK cells (FIG. 13B).

Interestingly, significant differences are observed between DC-induced expansion of NK cells and OC-induced NK cell expansion. Whereas OC-induced expansion of NK cells increased CD8+ T cell expansion, DC-induced expansion of NK cells resulted in expansion of CD4+ T cells. At the moment, the mechanisms governing the differential expansion of CD4+ vs. CD8+ T cells by DC- vs. OC-expanded NK cells respectively are not fully understood. However, there is larger increases in percentages of CD45RO and a higher decrease in percentages of CD62L surface expressions in T cells expanded by OC-expanded NK cells than DC-expanded NK cells indicating increased activation of T cells by the NK cells (FIG. 36L).

The higher activation signals by the OC-expanded NK cells are necessary for greater expansion of CD8+ T cells than CD4+ T cells. Indeed, OC-induced expansion of CD8+ T cells when total CD3+ T cells were used for expansion resulted in moderate increase in the expansion of CD8+ T cells and in the slight decline of CD4+ T cells (FIGS. 13A and 37A). Therefore, signals from both OCs and NK cells appear to be important in expansion and activation of CD8+ T cells, although the effect of NK cells appears to be more dominant than OCs. In addition, NK cells expanded by OCs have greater cytotoxic activity than those expanded by the DCs potentially providing the mechanism for targeting of CD4+ T cells and sparing CD8+ T cells. In support of this observation we have also observed that NK cells differentially targeted activated CD4+ and CD8+ T cells (FIG. 38). The OC-expanded NK cells as well as IL-2+anti-CD16mAb treated NK cells but less primary IL-2 activated NK cells were able to target activated CD4+ T cells (FIG. 38). Indeed, it has previously been shown that NK cells inhibit proliferation of CD4+ T cells under chronic antigen stimulation in the model of GVHD through Fas receptor and not perforin mediated killing, and that the lysis was mediated through the NKG2D ligand expression. In agreement, CD56^{bright} NK cells in comparison to CD56^{dim} subset were found to have higher degranulation and lysis of activated CD4+ T cells. CD56^{bright} NK cells were previously shown to have higher secretion of cytokines in the presence of no or lower cytotoxicity similar to those found with IL-2+anti-CD16mAb treated NK cells which we have previously coined as split anergized NK cells. Therefore, it is possible that the underlying mechanisms of CD4+ T cell lysis is through their death receptors triggered by Fas ligand, TNF-α and TRAIL on NK cells. Indeed, both split anergized NK cells and OC-expanded NK cells have very high induction of Fas ligand and TNF-α (FIG. 41). However, since super-charged NK cells have also significantly higher granule content with potent cytotoxic function, the granule mediated lysis of CD4+ T cells can't be ruled out in our system at present. Furthermore, it was also shown that upon stimulation with antigen and co-stimulatory signals CD4+ T cells undergo activation induced cell death through Fas receptors whereas CD8+ T cells are rendered non-responsive but gain function when IL-2 is provided. Therefore, there are clear differences between CD4+ and CD8+ T cell subsets in their susceptibility to cell death and mode of expansion. Thus, greater expansion of CD8+ T cells by both OCs and NK cells suggests increased selection as well as expansion of CD8+ T cells since NK cells select and also trigger expansion of CD8+ T cells. On the other hand, it appears that OCs will only aid moderately in expansion since these cells were not shown to have cytotoxic capability.

Although OCs were able to expand CD8+ T cells somewhat, the expansion of these cells were significantly accelerated in the presence of OC-expanded NK cells (FIGS. 13A-13B and 37A-37B). Therefore, there could be potentially two different mechanisms of CD8+ T cell expansion by the OC-expanded NK cells. One mechanism is likely contributed by the OCs in the initial phases of expansion where there still remains some OCs in the NK cultures which could be approximately up until day 6 or maximum 9 of expansion with fewer or minor expansion of CD8+ T cells. By day 9 no OCs are remaining in the culture of NK cells and therefore, there are only expanding super-charged NK cells with more rapidly expanding CD8+ T cells. Thus, the second mechanism is contributed by the super-charged NK cells which are likely through targeting of remaining CD4+ T cells and selection of CD8+ T cells and activation of CD8+ T cells. Although there are significant numbers of CD4+ T cells remaining after OC-mediated expansion of T cells, in the presence of OC-expanded NK cells the majority if not all are primarily CD8+ T cells (FIG. 13A). As for OC-expanded NK mediated expansion of CD8+ T cells, a significantly higher activation of CD8+ T cells in terms of increased percentage of cells expressing CD45RO and lower percentage of cells expressing CD62L is seen with OC-expanded NK cells when compared to just OC activated T cells (FIGS. 36L-36M), therefore, it is possible that either NK cells differentially target and kill activated CD4 cells and/or that activation induced cell death is higher in CD4+ T cells than it is in CD8+ T cells. The higher activation of CD8+ T cells by the NK cells in comparison to OC-induced CD8+ T cells is also seen when different cytokine levels were assessed (FIG. 15).

The patients are found to have on average higher percentages of CD8+ T cells in their peripheral blood when compared to those obtained from healthy individuals (FIGS. 35B-35C). When OC-derived from healthy individuals were used to expand both healthy and patient T cells, OC-expanded T cells also demonstrated higher percentages of CD8+ T cell expansion from both healthy and patient derived T cells, which were higher than those seen from those obtained initially from the peripheral blood (FIGS. 35B, 35C, 13A and 13B). Thus, the higher expansion seen with patients' OC-expanded T cells is likely due to the higher frequencies of CD8+ T cells in the patients when compared to healthy individuals. Indeed, on average 1 percent primary CD8+ T cells when expanded by healthy OCs will give rise to 1.2 percent expanded CD8+ T cells by patient T cells, but that average is at 1.6 percent with T cells from healthy individuals, which is higher. Thus, anti-CD3/CD28 activation and signaling through T cells augments the percentages of CD8+ T cells moderately, and the levels of expansion are less by patients' CD8+ T cells when compared to CD8+ T cells expanded from healthy individuals (FIGS. 13A-13B). In addition, OC-expanded NK cells expanded 2.73 percent CD8+ T cells from 1 percent of CD8+ T cells from healthy individuals, whereas from patients those percentages remained lower at 1.75 percent which on average is an almost one percentage point difference (FIGS. 35B, 35C, 13A and 13B). Thus, the extent of CD8+ T cell selection and expansion by OC-expanded NK cells is much higher in healthy individuals than those obtained from patients. Based on these results, it is evident that the selection and expansion of CD8+ T cells from both OC-expanded T and OC-expanded NK cells is significantly inferior by the patient cells when compared to those of healthy individuals.

In a second series of experiments we determined the rate of OC mediated CD8+ T cell expansion from both healthy and cancer patients using autologous OCs. Patients' OCs had lower ability to expand autologous CD8+ T cells from OC-expanded T and NK cells and the percentages of expansion were much less when compared to CD8+ expansion from OC expanded T and NK cells from healthy donors in an autologous system (FIG. 42). In addition, when the numbers of expanded NK cells and the levels of cytotoxicity and secretion of IFN-γ were assessed in an autologous system much lower levels of expansion, cytotoxicity and IFN-γ secretion could be observed from patient OC-expanded NK cells when compared to OC-expanded NK cells from healthy individuals (FIGS. 34E-34G). Using patient OCs for the expansion of NK cells from healthy individuals or healthy OCs with patient NK cells, we observed much lower expansion and function when compared to those obtained from OC-expanded NK cells in healthy individuals in an autologous system (FIG. 34). The lowest levels of expansion and function compared to the three groups mentioned above were seen when patient OCs were used to expand autologous NK cells, indicating that there are functional deficiencies in both NK and OCs from cancer patients, when compared to the function of both cell types from healthy individuals. Indeed, when comparing the surface receptor expression between patient and healthy OCs, there is significant down-modulation of activating receptors which could be one reason why the patient OCs may not support the expansion of allogeneic or autologous NK and CD8+ T cells. However, we also see a substantial decrease in MHC-class I inhibitory signals which should provide an activating signal due to decrease binding and inhibition of NK cells through MHC-class I inhibitory receptors. It appears that most receptors are down-modulated on the surface of patient OCs irrespective of whether they are activating or inhibitory ligands for the NK cells. In this scenario it is possible that lack of activating ligands supersedes the effect of lack of inhibitory ligands since OCs from patients are not able to activate autologous NK cells.

In agreement with our in vitro studies, injection of OC-expanded NK cells to tumor-bearing hu-BLT mice increased the numbers of CD8+ T cells in BM, spleen, and peripheral blood resulting in the increased levels of NK cell-mediated cytotoxicity as well as increased secretion of IFN-γ (FIGS. 14B-14J). Increased levels of IFN-γ, IL6, ITAC were also observed in the sera of tumor-bearing hu-BLT mice injected with OC-expanded NK cells (FIG. 14K).

Potential relevance of our observations could be seen in the studies reported with multiple myeloma (MM) patients. Indeed, these patients have multifocal neoplastic proliferation of monoclonal plasma cells in the bone marrow where significant numbers of OCs reside. It was shown that these patients had higher levels of NK and CD8+ T cells in both peripheral blood and bone marrow aspirates when compared to heathy controls. It was also found that the ratio of CD4/CD8 was decreased in the patients and this decrease was co-related with an increase in human leukocyte antigen (HLA)-DR expression by CD8+ but not CD4+ T cells. Moreover, it was noted that patients with long-term disease control exhibited an expansion of cytotoxic CD8+ T cells and natural killer cells. T cell expansions in MM patients have a phenotype of cytotoxic T cells, with expanded V-beta TCR populations having predominantly CD8+, CD57+, CD28− and perforin+ phenotype. Our observations are relevant to MM patients since they exhibit significant BM pathology, and it is also likely that the mechanisms discussed herein also occur in patients who may sustain bone metastasis or have primary tumors inflicting bone.

### Example 29: Material and Methods for Example 30

Sera Collection from Human Donors and Hu-BLT Mice Peripheral Blood

Peripheral blood (200 μl) was collected in 1.5 ml heparin-free Eppendorf tubes and left in room temperature for 15-20 minutes. The tubes were then centrifuged at 2000 rpm for 10 mins, and the sera were then harvested.

TABLE 1
Participating cancer patients

| | Gender | Cancer type | Cancer stage (TNM) |
|---|---|---|---|
| Patient 1 | M | Oral | Stage 4 |
| Patient 2 | M | Oral | Stage 4 |
| Patient 3 | M | Oral | Stage 4 |
| Patient 4 | F | Pancreatic | Stage 4 |
| Patient 5 | M | Pancreatic | Stage 4 |
| Patient 6 | F | Pancreatic | Stage 4 |
| Patient 7 | F | Pancreatic | Stage 4 |
| Patient 8 | F | Pancreatic | Stage 4 |
| Patient 9 | M | Pancreatic | Stage 4 |
| Patient 10 | M | Pancreatic | Stage 4 |
| Patient 11 | M | Pancreatic | Stage 4 |
| Patient 12 | M | Pancreatic | Stage 4 |
| Patient 13 | M | Colon | Stage 4 |
| Patient 14 | M | Colon | Stage 4 |
| Patient 15 | M | Colon | Stage 4 |
| Patient 16 | M | Prostate | Stage 4 |

### Example 30: OC-Expanded NK Cells Secreted More Cytokines and Chemokines when Compared to Expanded T Cells

NK cells were cultured with OCs for 12 days before expanded NK cells, and NK cell expanded CD8+ T cells were isolated from the same culture. Isolated NK cells were treated with a combination of IL-2 and anti-CD16 mAb, and NK expanded CD8+ T cells were treated with IL-2 and anti-CD3/CD28 mAb for 18 hours before the supernatants were harvested from the cultures and secretions were assessed using multiplex arrays. We compared the amounts secreted by the NK cells with the amounts which were secreted by the NK-expanded CD8+ T cells, and determined the fold increase in NK cells when compared to NK expanded CD8+ T cells. NK cells secreted higher levels of all cytokines and chemokines with the exception of IL-3 which was lower by the NK cells than NK-expanded CD8+ T cells as shown in the Figure S4A. In particular NK cells secreted higher levels of secreted CD137, secreted Fas-Ligand (sFasL), Granzyme A and B, IL-10, TNF-α, MIP1-1a, and MP1b, when compared to CD8+ T cells (FIG. 41A). OC-expanded NK cells produced more abundant amounts of GM-CSF, soluble CD137, IFN-γ, soluble Fas, sFasL, perforin, MIP-1a, and MP1b, while OC-expanded T cells readily produced IL-10, granzymes A and B, and TNF-α during the expansion periods (FIGS. 41B-41C). Then, we examined the secreted factors from CD8+ T cells isolated from day 12 OC-expanded NK cell cultures and compared to purified CD8+ T cells expanded by OC only. CD8+ T cells isolated from the OC-expanded NK cell cultures secreted higher levels of GM-CSF, soluble CD137, IFN-γ, IL-10, sFasL, and TNF-α but lower levels of granzyme A and perforin; and similar levels of granzyme B and soluble Fas, when compared to OC expanded CD8+ T cells (FIG. 15).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

1. Jewett, A., A. Arasteh, et al. “Strategies to Rescue Mesenchymal Stem Cells (MSCs) and Dental Pulp Stem Cells (DPSCs) from NK Cell Mediated Cytotoxicity.” PLoS One. 2010 Mar. 31; 5(3):e9874.
2. Han-Ching Tseng, Aida Arasteh, Avina Paranjpe, Antonia Teruel, Wendy Yang, Armin Behel, Jackelyn Alva, Walters Gina, Christian Head, Tomo-o Ishikawa, Harvey Herschman, Nicholas Cacalano, April Pyle, No-Hee Park, and Anahid Jewett, Increased lysis of stem cells but not their differentiated cells by Natural Killer cells; De-differentiation or reprogramming activates NK cells, PLoS One. 2010 Jul. 16; 5(7):e11590
3. JOHN A. YAGIELA AND ANAHID JEWETT, Pharmacology and Therapeutics for Dentistry, Immunotherapy, Chapter 41, 2010
4. Jewett, A. and H. C. Tseng, Tumor induced inactivation of natural killer cell cytotoxic function; implication in growth, expansion and differentiation of cancer stem cells. J Cancer. 2011; 2:443-57. Epub 2011 Aug. 10.
5. Anahid Jewett, Han-Ching Tseng, Aida Arasteh, Saba Saadat, Russell E. Christensen and Nicholas A. Cacalano. Natural Killer cells preferentially target cancer stem cells; Role of monocvtes in protection against NK cell mediated lysis of cancer stem cells. Curr Drug Deliv., 2011, Oct. 21. [Epub ahead of print]
6. Jewett, A. and H. C. Tseng, Potential rescue, survival and differentiation of cancer stem cells and primary non-transformed stem cells by monocyte-induced split anergy in natural killer cells. Cancer Immunol Immunother. 2012 February; 61(2):265-74. Epub 2011 Nov. 25
7. Anahid Jewett*, Hiromi Nakamura, Meiying Wang, Antonia Teruel, Avina Paranjpe and Marcela Romero, Dedifferentiation of Epithelial Tumors Enhances Cytotoxicity, Survival and Expansion of Allogeneic CD8+ T Cells and Natural Killer Cells; J Carcinogen Mutagen 2012, S1, http://dx.doi.org/4172/2157-2518.S1-007
8. Anahid Jewett, Yan-Gao Man, Han-Ching Tseng, Dual functions of Natural Killer cells in selection and differentiation of stem cells; Role in regulation of inflammation and regeneration of tissues. J Cancer. 2013; 4(1):12-24. doi: 10.7150/jca.5519. Epub 2012 Dec. 1
9. Anahid Jewett and Han-Ching Tseng, Tumor microenvironment may shape the function and phenotype of NK cells through the induction of split anergy and generation of regulatory NK cells. Chapter 15 “The Tumor microenvironment” Edited by Drs. Michael R. Shurin, Viktor Umansky and Anatoli Malyguine, 2013
10. Jewett A. Special issue on immune responses in tumors and non-transformed inflammatory microenvironments. J Cancer. 2013; 4(1):1-2. doi: 10.7150/jca.5045. Epub 2012 Nov. 30. PMID:23386899
11. Tseng HC, Bui V, Man Y G, Cacalano N, Jewett A. Induction of Split Anergy Conditions Natural Killer Cells to Promote Differentiation of Stem Cells through Cell-Cell Contact and Secreted Factors. Front Immunol. 2014 Jun. 19; 5:269. doi: 10.3389/fimmu.2014.00269. eCollection 2014. PMID: 24995006, [PubMed]
12. Perisic Nanut M, Sabotic J, Jewett A, Kos J. Cysteine cathepsins as regulators of the cytotoxicity of NK and T cells. Front Immunol. 2014 Dec. 2; 5:616. doi: 10.3389/fimmu.2014.00616. eCollection 2014. Review. PMkID. 25271
13. Shurin M R, Umansky V, Malyguine A, Hurwitz A A, Apte RN, Whiteside T, Jewett A, Thanavala Y, Murphy WJ. Cellular and molecular pathways in the tumor immunoenvironment: 3rd Cancer Immunotherapy and Immunomonitoring (CITIM) meeting, 22-25 Apr. 2013, Krakow, Poland. Cancer Immunol Immunother. 2014 January; 63(1):73-80. doi: 10.1007/s00262-013-1501-z. Epub 2013 November 24. PMID: 24271210
14. Jewett A, Man Y G, Cacalano N, Kos J, Tseng HC. Natural killer cells as effectors of selection and differentiation of stem cells: Role in resolution of inflammation. [Published online ahead of print Feb. 27, 2014] J Immunotoxicol doi: 103109/1547691X2013877104.
15. Tseng, H., Cacalano, N., & Jewett, A. (2015). Split anergized Natural Killer cells halt inflammation by inducing stem cell differentiation, resistance to NK cell cytotoxicity and prevention of cytokine and chemokine secretion. Oncotarget, 6(11), 8947-8959.
16. Park S, Kanayama K, Kaur K, Tseng HC, Banankhah S, Quje D T, Sayre J W, Jewett A, Nishimura I. Osteonecrosis of the Jaw Developed in Mice: DISEASE VARIANTS REGULATED BY γδ T CELLS IN ORAL MUCOSAL BARRIER IMMUNITY. J Biol Chem. 2015 Jul. 10; 290(28):17349-66. doi: 10.1074/jbc.M115.652305. Epub 2015 May 26.P D26013832
17. Tseng HC, Arasteh A, Kaur K, Kozlowska A, Topchyan P, Jewett A. Differential Cytotoxicity but Augmented IFN-γ Secretion by NK Cells after Interaction with Monocytes from Humans, and Those from Wild Type and Myeloid-Specific COX-2 Knockout Mice. Front Immunol. 2015 Jun. 9; 6:259. doi: 10.3389/fimmu.2015.00259. eCollection 2015. PID 26101 6386
18. Magister, S., Tseng, H., Bui, V., Kos, J., & Jewett, A. (2015). Regulation of split anergy in natural killer cells by inhibition of cathepsins C and H and cystatin F. Oncotarget, 5. 2015 Sep. 8; 6(26):22310-27
19. Han-Ching Tseng^{1&}; Keiichi Kanayama^{2,3&}; Kawaljit Kaurl&; So-Hyun Park^{1&}, Sil Park^{1,2}; Anna Kozlowska⁴; Shuting Sun⁵, Charles E. McKenna⁵, Ichiro Nishimura**^{1,2} and Anahid Jewett*¹. Bisphosphonate-induced differential modulation of immune cell function in gingiva and bone marrow in vivo; Role in osteoclast-mediated NK cell activation. Oncotarget, 2015 Aug. 21; 6(24):20002-25
20. Han-Ching Tseng, Akihito Inagaki, Vickie T. Bui, Nicholas Cacalano, Noriyuki Kasahara, Yan-gao Man and Anahid Jewett, Differential Targeting of Stem Cells and Differentiated Glioblastomas by NK Cells, Journal of Cancer, 2015; 6(9): 866-876. doi: 10.7150/jca.11527
21. Bui, V. T., H.-C. Tseng, P. O. Maung, A. Kozlowska, K. Mann, P. Topchyan and A. Jewett. “Augmented IFN-γ and TNF-α Induced by Probiotic Bacteria in NK Cells Mediate Differentiation of Stem-Like Tumors Leading to Inhibition of Tumor Growth and Reduction in Inflammatory Cytokine Release; Regulation by IL-10.” Frontiers in Immunology. 6:576.doi: 10.3389/fimmu. 2015,00576
22. Rezzadeh K S, Hokugo A, Jewett A, Kozlowska A, Segovia L A, Zuk P, Jarrahy R. Natural Killer Cells Differentiate Human Adipose-Derived Stem Cells and Modulate Their Adipogenic Potential. Plast Reconstr Surg. 2015 September; 136(3):503-10. doi: 10.1097/PRS.0000000000001536. PMID: 26313823
23. Marcela Romero Reyes and Anahid Jewett, Increased activation and survival of immune effectors during anti-TNF-α therapy: Lessons learned from TNF−/− mice; New Approaches combating Cancer & Aging 2015; 2: 76-88
24. ANAHID JEWETT and Han-Ching Tseng, Pharmacology and Therapeutics for Dentistry, 7e, Immunotherapy, Chapter 35, 2015
25. Natalia Aptsiauri, Anahid Jewett, Arthur A. Hurwitz, Michael R. Shurin, Viktor Umansky. Redefining cancer immunotherapy—optimization, personalization and new predictive biomarkers: 4th Cancer Immunotherapy and Immunomonitoring (CITIM) meeting, 27-30 Apr. 2015, Ljubljana, Slovenia, Cancer Immunology and Immunotherapy, 2016 July; 65(7):835-45. doi: 10.1007/s00262-016-1822-9. Epub 2016 Mar. 31. Review.
26. Kozlowska A K, Kaur K, Topchyan P, Jewett A, Adoptive transfer of osteoclast-expanded natural killer cells for immunotherapy targeting cancer stem-like cells in humanized mice. Cancer Immunol Immunother. 2016 July; 65(7):835-45. doi: 10.1007/s00262-016-1822-9. Epub 2016 Mar. 31. Review
27. Anna K. Kozlowska, Han-Ching Tseng, Kawaljit Kaur, Paytsar Topchyan, Akihito Inagaki, Vickie T. Bui, Noriyuki Kasahara, Nicholas Cacalano and Anahid Jewett Resistance to cytotoxicity and sustained release of interleukin-6 and interleukin-8 in the presence of decreased interferon-g after differentiation of glioblastoma by human natural killer cells; Cancer Immunology and Immunotherapy; 2016; 65(9): 1085-97
28. Anna K. Kozlowska, Kawaljit Kaur, Paytsar Topchyan, Anahid Jewett, Novel strategies to target cancer stem cells by NK cells; studies in humanized mice; Frontiers in bioscience (Landmark edition), 2017; 22: 370-384.
29. Yujie Sun, Kawaljit Kaur, Keiichi Kanayama, Kenzo Morinaga, Sil Park, Akishige, Hokugo, Anna Kozlowska, William H. McBride, Jun L1, Anahid Jewett, Ichiro Nishimura. Plasticity of Myeloid Cells during Oral Barrier Wound Healing and the Development of Bisphosphonate-Related Osteonecrosis of the Jaw. J Biol Chem. 2016 Aug. 11. pii: jbc.M116.735795. [Epub ahead of print]
30. Kaur K, Cook J, Park S, Topchyan P, Kozlowska A, Ohanian N, Fang C, Nishimura I and Jewett A (2017). Novel strategy to expand super-charged NK cells with significant potential to lyse and differentiate cancer stem cells; Differences in NK expansion and function between healthy and cancer patients. Front. Immunol 7:9. doi: 10.3389/fonc.2017.00009
31. Anna Kozlowska, Paytsar Topchvan, Kawaljit Kaur, Han-Ching Tseng, Antonia Teruel, Toru Hiraga, and Anahid Jewett, Differentiation by NK cells is a prerequisite for effective targeting of cancer stem cells/poorly differentiated tumors by chemopreventive and chemotherapeutic drugs; Journal of Cancer, 2017; 8(4): 537-554. doi: 10.7150/jca.15989
32. Milica M Perisic Nanut, Jerics Sabotie, Urban Svajger, Anahid Jewett, Janko Kos; Cystatin F affects natural killer cell cytotoxicity, “Frontiers in Immunology” 2017 November 13; 8:1459. doi: 10.3389/fimmu.2017.01459. eCollection 2017.
33. Kawaljit Kaur1, Hui-Hua Chang1, Jessica Cook1, Guido Eibl1, Anahid Jewett; Suppression of Gingival NK Cells in Precancerous and Cancerous Stages of Pancreatic Cancer in KC and BLT-Humanized Mice. “Frontiers in Immunology” 2017 Dec. 4; 8:1606. doi: 10.3389/fimmu.2017.01606. eCollection 2017.
34. Kaur, K., P. Topchyan, A. K. Kozlowska, N. Ohanian, J. Chiang, P. O. Maung, S.-H. Park, M.-W. Ko, C. Fang, I. Nishimura and A. Jewett (2018). “Super-charged NK cells inhibit growth and progression of stem-like/poorly differentiated oral tumors in vivo in humanized BLT mice; effect on tumor differentiation and response to chemotherapeutic drugs. Oncoimmunology. 2018 Feb. 22; 7(5):e1426518. doi: 10.1080/2162402X.2018.1426518. eCollection 2018
35. Jewett A. Editorial overview: Tumour immunology: Are we on the path to win the battle against cancer through immunotherapy? Curr Opin Immunol. 2018 April; 51:vii-ix. doi: 10.1016/j.coi.2018.04.018.
36. Kawaljit Kaur¹, Milica Perisid Nanut², Mengwei Kol, Tahmineh Safaie¹, Janko Kos^{2,3} and Anahid Jewett^{1,4}* Natural killer cells target and differentiate cancer stem-like cells/undifferentiated tumors: strategies to optimize their growth and expansion for effective cancer immunotherapy; Curr Opin Immunol. 2018 April; 51:170-180. doi: 10.1016/j.coi.2018.03.022. Epub 2018 Apr. 10.
37. Janko Kos1,2 Milica Perišić Nanut1 ⋅Mateja Prunkl Jerica Sabotič1 Esmeralda Dautovič3⋅Anahid Jewett4. Cystatin F as a regulator of immune cell cytotoxicity; Cancer Immunology and Immunotherapy; https://doi.org/i0.1007/s00262-018-2165-5

We claim:

1. A method of assessing the function of NK cells, comprising:
a) assessing the cytotoxic function of the NK cells against cancer cells and/or cancer stem cells;
b) assessing the amount of interferon-γ (IFN-γ) produced by the NK cells; and
c) assessing the ability of IFN-γ produced by the NK cells to induce differentiation of tumor cells.

2. The method of claim 1, wherein the cancer cells or cancer stem cells in a) are oral squamous cancer stem cell (OSCSC) and/or Mia-Paca-2 (MP2) cells.

3. The method of any preceding claim, wherein assessing the amount of IFN-γ produced by the NK cells comprises:
i) assessing the amount of interferon-γ (IFN-γ) produced by a population of the NK cells or all NK cells in a culture; and
ii) assessing the amount of IFN-γ produced by the NK cells at a single-cell level.

4. The method of claim 3, wherein the amount of IFN-γ produced by the population of the NK cells or all NK cells in the culture in i) is measured by an ELISA assay.

5. The method of claim 3 or 4, wherein the amount of IFN-γ produced by the NK cells at the single-cell level in ii) is determined by an ELISPOT assay.

6. The method of any preceding claim, wherein assessing the cytotoxic function of the NK cells comprises assessing direct cytotoxic killing by the NK cells.

7. The method of any preceding claim, wherein assessing the cytotoxic function of the NK cells comprises assessing antibody-dependent cellular cytotoxicity (ADCC) activity of the NK cells.

8. The method of any preceding claim, wherein the cytotoxic function of the NK cells is measured using a ⁵¹Cr release cytotoxicity assay.

9. The method of any preceding claim, wherein the ability of the IFN-γ to induce differentiation of tumor cells is assessed by incubating tumor cells with IFN-γ, wherein:
a) if the IFN-γ decreases and/or inhibits the tumor growth and/or tumor cell division as compared to the tumor growth and/or tumor cell division in the control, the IFN-γ is able to induce differentiation of the tumor cells;
b) if the IFN-γ decreases an expression level of CD44 and/or increases an expression level of at least one of CD54, MHC class I, and PD-L1 as compared to the expression level of the same markers in the control, the IFN-γ is able to induce differentiation of the tumor cells; and/or
c) if the IFN-γ increases resistance of the tumor cells to the NK-cell-mediated cytotoxicity as compared to the resistance in the control, the IFN-γ is able to induce differentiation of the tumor cells.

10. The method of any preceding claim, wherein the NK cells are determined to have substandard cytotoxicity if
a) the efficiency at which the NK cells directly kill cancer cells and/or cancer stem cells is less than about 60%, 70%, or 80% of the efficiency at which healthy NK cells directly kill cancer cells and/or cancer stem cells,
b) at least one NK cell is needed to mediate direct killing of one cancer cell and/or cancer stem cell (i.e., one NK cell is not able to kill more than one cancer cell),
c) the efficiency at which the NK cells mediate ADCC-dependent killing of cancer cells and/or cancer stem cells is less than about 60%, 70%, or 80% of the efficiency at which healthy NK cells mediate ADCC-dependent killing of cancer cells and/or cancer stem cells, and/or
d) at least one NK cell is needed to mediate ADCC-dependent killing of one cancer cell and/or cancer stem cell (i.e., one NK cell is not able to kill more than one cancer cell),
optionally wherein the efficiency is determined for a period of less than about 0.5, 1, 2, 3, 4, 5, 6, 7, 8, 9, 10, 11, or 12 hours of co-incubation of the NK cells with cancer cells and/or cancer stem cells.

11. The method of any preceding claim, wherein the NK cells are determined to have substandard levels of IFN-γ secretion if:
a) (i) the amount of IFN-γ produced by the NK cells when treated with IL-2 is less than about 60%, 70%, or 80% of the amount of IFN-γ produced by healthy NK cells when treated with IL-2, or
(ii) the amount of IFN-γ produced by each million NK cells when treated with IL-2 is less than about 300 μg; and/or
b) the NK cells produce less than about 60%, 70%, or 80% of the amount of IFN-γ produced by healthy NK cells at the single cell level.

12. The method of any preceding claim, wherein the NK cells are determined to have substandard IFN-γ tumor differentiation potency if the IFN-γ produced by the NK cells (“test IFN-γ”):
i) does not decrease or inhibit tumor growth and/or tumor cell division by at least 10%, 20%, 30%, or 40%;
ii) does not decrease the expression level of CD44 on tumor cells by at least about 10%, 20%, 30%, or 40%;
iii) does not increase the expression level of at least one of CD54, MHC class I, and PD-L1 on tumor cells by at least about 10%, 20%, 30%, or 40%; and/or
iv) does not increase resistance of the tumor cells to NK-cell-mediated cytotoxicity by at least 10%, 20%, 30%, or 40%.

13. The method of any one of the preceding claim, further comprising assessing the ability of the NK cells to be expanded by the osteoclast cells.

14. The method of claim 13, wherein the osteoclast cells are autologous or allogeneic relative to the NK cells.

15. The method of claim 13 or 14, wherein assessing the ability of the NK cells to be expanded by the osteoclast cells comprises culturing the NK cells in a medium together with the osteoclast cells.

16. The method of any one of claims 13-15, wherein assessing comprises expanding the NK cells to at least about 10, 11, 12, 13, 14, 15, 16, 17, 18, 19, 20, 21, 22, 23, 24, 25, or more population doublings within 4 weeks.

17. The method of any one of claims 13-16, wherein the NK cells are determined to have substandard expansion potential if the NK cells are not expanded by osteoclast cells to at least about 10, 11, 12, 13, 14, 15, 16, 17, 18, 19, 20, 21, 22, 23, 24, 25, or more population doublings within 4 weeks.

18. The method of any one of the preceding claims, further comprising assessing the ability of the NK cells to expand CD8+ T cells.

19. The method of claim 18, wherein the ability of the NK cells to expand the CD8+ T cells is determined relative to the ability of the NK cells to expand CD4+ T cells.

20. The method of claim 18 or 19, wherein the NK cells are determined to have substandard ability to expand CD8+ T cells if the autologous NK cells do not expand CD8+ T cells to at least 2-, 3-, 4-, 5-, 6-, 7-, 8-, 9-, 10-fold or more.

21. The method of any preceding claim, further comprising assessing the amount and/or function of a CD16 receptor on the NK cells.

22. The method of claim 21, wherein the function of the CD16 receptor on the NK cells is determined by assessing the NK cells' ability to (a) secrete IFN-γ and/or (b) mediate ADCC function against differentiated tumors, in response to CD16.

23. The method of claim 21, wherein the NK cells are determined to have substandard CD16 expression if the NK cells have a decrease in CD16 expression by at least about 10%, 15%, 20%, 25% or more relative to healthy NK cells.

24. The method of any preceding claim, wherein, if the NK cells are determined to be substandard for one or more functions, the method further comprises activating the NK cells.

25. The method of claim 24, wherein activating the NK cells comprises contacting the NK cells with monocytes expressing an amount of CD16 sufficient to activate the NK cells.

26. The method of claim 25, wherein the monocytes are autologous or allogeneic relative to the NK cells.

27. The method of claim 25 or 26, wherein the monocytes comprise an exogenous nucleic acid encoding CD16 or an active fragment thereof.

28. The method of any one of claims 24-27, wherein activating the NK cells comprises contacting the NK cells with at least one agent selected from IL-2, CD16, anti-CD16 antibody, anti-CD3 antibody, anti-CD28 antibody, and a composition comprising at least one bacterial strain.

29. The method of claim 28, wherein the composition comprises at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated.

30. The method of claim 28 or 29, wherein the composition comprises sAJ2 bacteria.

31. The method of any preceding claim, further comprising expanding the NK cells by contacting the osteoclast cells and/or dendritic cells, optionally wherein the osteoclast cells and/or dendritic cells are autologous or allogeneic to the NK cells.

32. The method of claim 31, wherein the osteoclast cell enhances the secretion of IL-12, IL-6, TNF-α, IL-5, and/or IL-4 by the NK cell.

33. The method of any preceding claim, wherein the NK cells are primary NK cells, optionally wherein the primary NK cells have not been transformed.

34. A method of determining whether NK cells are suitable for administration to a subject, such as a subject afflicted with cancer, comprising performing the method of any preceding claim on the NK cells.

35. A method of determining a suitable therapy for a subject, comprising performing the method of any one of claims 1-33 on NK cells obtained from the subject.

36. The method of claim 35, wherein the subject is a subject afflicted with cancer and the method further comprises determining the amount of at least one of CD44, CD54, MHC class I, PD-L1 (B7H1), MICA, and MICB on cancer cells of the subject.

37. The method of claim 35 or 36, wherein the NK cells are determined to have standard cytotoxicity but substandard production of IFN-γ.

38. The method of claim 37, wherein the suitable therapy is IL-2 and/or a probiotic composition.

39. The method of claim 35 or 36, wherein the NK cells are determined to have standard IFN-γ production but are substandard in ability to expand CD8+ T cells, cytotoxic function, and/or expansion potential.

40. The method of claim 39, wherein the suitable therapy is IL-2, IL-15, and/or IL-21, and/or a probiotic composition.

41. The method of claim 40, wherein the suitable therapy further comprises chemotherapy or radiotherapy.

42. The method of claim 38 or 40, wherein the probiotic composition comprises at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated.

43. The method of claim 42, wherein the probiotic composition comprises sAJ2 bacteria.

44. The method of any one of claims 34-43, wherein the method further comprises administering the suitable therapy to the patient.

45. The method of any one of claims 34-44, further comprising administering to the subject the NK cell-expanded CD8+ T cells.

46. The method of any one of claims one of claims 34-45, further comprising administering an immunotherapy other than the NK cell-based immunotherapy, if the subject's NK cells show a low amount and/or function of the CD16 receptor.

47. The method of any one of claims 34-46, wherein the subject is mammal.

48. The method of claim 47, wherein the mammal is a mouse or a human.

49. The method of claim 47 or 48, wherein the mammal is a human.

50. A kit for assessing the function of NK cells, wherein the kit comprises a combination of at least one reagent for performing each of the assessments in (a)-(c) of claim 1, as further defined in any of claims 2-9.

51. The kit of claim 50, further comprising at least one reagent for performing the assessments in any one of claims 13, 15, 18, 19, 21, 22, and 24-33.
